(19) United States
(12) Patent Application Publication
(10) Pub. No.: US 2025/0281639 A1
(43) Pub. Date: Sep. 11, 2025

(54) COMPOSITIONS AND METHODS FOR TREATING CHRONIC PAIN AND FOR RETROGRADE TRANSDUCTION OF NEURONS

(71) Applicant: The Board of Trustees of the Leland Stanford Junior University, Stanford, CA (US)

(72) Inventors: Xiaoke Chen, Redwood City, CA (US); Qian Wang, Redwood City, CA (US); Greg Nachtrab, Redwood City, CA (US)

(21) Appl. No.: 18/577,964

(22) PCT Filed: Jul. 8, 2022
(86) PCT No.: PCT/US2022/073560

§ 371 Date: Jan. 9, 2024

Related U.S. Application Data
(60) Provisional application No. 63/221,759, filed on Jul. 14, 2021.

(51) Int. Cl. A61K 48/00; A61P 25/02; C07K 14/005; C12N 9/22; C12N 15/11; C12N 15/113; C12N 15/86

(52) U.S. Cl. CPC A61K 48/005; A61P 25/02; C07K 14/005; C12N 9/222; C12N 15/111; C12N 15/113; C12N 15/86; C12N 2310/20; C12N 2310/531; C12N 2750/14122; C12N 2750/14143

(57) ABSTRACT

Methods and compositions are provided for treating an individual in need (e.g., one who has chronic pain). Such methods can include a step of administering a therapy that reduces CamKv activity in opioid receptor mu 1 (OPRM1) expressing neurons of the individual's rostral ventromedial medulla (RVM). In some cases, the therapy (e.g., deep brain stimulation of zona incerta neurons) increases inhibitory input into the individual's RVM. In some cases, the therapy is an agent (e.g., an RNAi agent) that reduces expression or activity of CamKv in the individual's RVM. In some cases, the agent includes a retrograde-enhanced recombinant AAV particle, e.g., one that can be used to deliver an RNAi agent such as an shRNA that targets CamKv. Also provide are retrograde-enhanced clade E variant AAV capsid proteins, AVV particles that include such capsid proteins, methods of making such AAV particles, and methods of expressing a transgene using such AAVs.

### CROSS-REFERENCE

This application claims the benefit of U.S. Provisional Patent Application No. 63/221,759 filed Jul. 14, 2021, which application is incorporated herein by reference in its entirety.

### STATEMENT REGARDING FEDERALLY SPONSORED RESEARCH

This invention was made with Government support under contracts DA045664 and MH116904 awarded by the National Institutes of Health. The Government has certain rights in the invention.

### INCORPORATION BY REFERENCE OF SEQUENCE LISTING PROVIDED AS AN XML FILE

A Sequence Listing is provided herewith as a Sequence Listing XML, “STAN-1842_SEQ_LIST_AUG_2024.xml” created on Aug. 8, 2024 and having a size of 97,119 bytes. The contents of the Sequence Listing XML are incorporated by reference herein in their entirety.

### INTRODUCTION

Tissue injury or inflammation usually transiently sensitizes pain which attracts attention to prevent further damage and promote healing. However, sometime this sensitization persists, leading to chronic pain that imposes tremendous psychological and socioeconomic burdens. Opiates have been used for centuries as potent analgesics, but issues with tolerance, abuse, and overdose make long-term prescription of opiates for chronic pain problematic. On the other hand, it is well documented that the level of perceived pain can be strongly influenced by cognition and mood, implying the existence of powerful endogenous top-down modulation of pain. Attaining a better understanding of descending pain modulation pathways could help identify novel targets for non-opiate treatment of chronic pain.

Thus, there is a need for compositions and methods that provide for treating individuals who suffer from chronic pain, and such is provided herein.

### SUMMARY

The work described in the experimental examples below led to the surprising finding by the inventors that the lateral superior colliculus rather than the traditionally assumed periaqueductal gray, provides the excitatory input onto the OPRM1⁺ RVM^{SC} neurons that drive mechanical hypersensitivity. Combining Ribotag RNA profiling and pathway manipulation, the work described below established that collicular inputs are essential for upregulating pseudokinase CaMKv in the OPRM1⁺ RVM^{SC} neurons after nerve injury, and demonstrated that up- or down-regulation of CaMKv is sufficient to drive or reverse mechanical hypersensitivity. Together, the results described below reveal a collicular-medulla-spinal cord pathway that drives persistent pain and identify CaMKv as a key molecular determinant of mechanical hypersensitivity.

Provided are methods of treating an individual in need (e.g., an individual who has chronic pain). Such methods can include a step of administering a therapy that reduces CamKv activity in opioid receptor mu 1 (OPRM1) expressing neurons of the individual's rostral ventromedial medulla (RVM). In some cases, the therapy is an agent (e.g., a biological agent such as an RNAi agent) that reduces expression of CamKv in said neurons. In some cases, the agent includes a retrograde-enhanced recombinant AAV particle (e.g., see below). In some cases, a subject retrograde-enhanced recombinant AAV particle is used to deliver an RNAi agent such as an shRNA that targets CamKv.

In some cases, the therapy comprises reducing excitatory input into the RVM from RVM projecting lateral superior colliculus (lSCIndG) neurons. In some cases, the therapy comprises increasing inhibitory input into the RVM (e.g., increasing inhibitory input into the RVM from zona incerta neurons). For example, inhibitory input can be controlled using deep brain stimulation to achieve pain suppression (e.g., by stimulating neurons of the zona incerta that provide inhibitory input to RVM neurons).

Moreover, the inventors have created a new viral capsid protein that provided a large and surprising increase in efficiency (about 2.3-fold) in retrograde labeling of neurons (e.g., rostral ventromedial medulla (RVM^{SC}) neurons) over the previous known AAV2-retro.

Thus, also provided are retrograde-enhanced clade E variant adeno-associated virus (AAV) capsid proteins, transduction systems that include nucleic acids that encode such capsid proteins, AAV viral particles that include such capsid proteins, methods of making viral particles that include such capsid proteins, and methods of expressing transgenes of interest using viral particles that include such capsid proteins. In some cases, a subject clade E variant capsid protein (CEVCP) includes the amino acid sequence LADQDYTKTA (SEQ ID NO: 30) inserted into a clade E AAV capsid protein. In some such cases, the LADQDYTKTA (SEQ ID NO: 30) sequence immediately follows a QQQN (SEQ ID NO: 28), QQTN (SEQ ID NO: 29), QQQD (SEQ ID NO: 30), or QQAN (SEQ ID NO: 31) sequence (see, e.g., FIG. 11). In some cases, a subject clade E variant capsid protein has an amino acid sequence having 85% or more sequence identity with the amino acid sequence set forth in any one of SEQ ID NOs: 32-57. In some cases, a subject clade E variant capsid protein has an amino acid sequence having 85% or more sequence identity with the AAV8-retro capsid amino acid sequence set forth in SEQ ID NO: 32 or with the rh10-retro capsid amino acid sequence set for in SEQ ID NO: 57. In some cases, a subject clade E variant capsid protein has the amino acid sequence LADQDYTKTA (SEQ ID NO: 30) inserted into an AAV8 capsid protein or into an rh10 capsid protein. In some cases, a subject clade E variant capsid protein is an AAV8-retro capsid protein comprising the amino acid sequence set forth in SEQ ID NO 32 or an rh10-retro capsid protein comprising the amino acid sequence set forth in SEQ ID NO 57.

As noted above, provided are transduction systems. Such systems include on or more nucleic acids, where said one or more nucleic acids comprises a nucleotide sequence that encodes a retrograde-enhanced clade E variant AAV capsid protein (e.g., any of the above-described retrograde-enhanced clade E variant AAV capsid proteins). In some cases, the one or more nucleic acids include a transgene sequence (e.g., encoding a protein such as a genome-targeting protein, e.g., a Zinc Finger or TALE or CRISPR/Cas effector protein, or encoding a non-coding RNA, e.g., an RNAi agent such as an shRNA or a CRISPR/Cas guide RNA, e.g., shRNAs or guide RNAs that target CamKv).

As noted above, provided are viral particles. Such viral particles can be retrograde-enhanced recombinant AAV particles that include a retrograde-enhanced clade E variant AAV capsid protein (e.g., any of the above-described retrograde-enhanced clade E variant AAV capsid proteins, e.g., one that includes LADQDYTKTA (SEQ ID NO: 30) inserted into an AAV8 capsid protein or into an rh10 capsid protein). In some cases, such particles include a nucleic acid that encodes a transgene sequence (e.g., where the transgene sequence can be flanked by ITRs). The transgene sequence can be any convenient sequence, e.g., see examples provided above with respect to transduction systems. As noted above, also provided are methods of making viral particles, such as those described herein. Such methods can include a step of introducing a subject transduction system (e.g., see above) into a eukaryotic cell such that the cell produces said retrograde-enhanced recombinant AAV particle. Such methods can also include a step of isolating AAV particles that are produced.

Also as noted above, provided are methods of expressing transgenes of interest. Such methods can include contacting a neuron with a subject retrograde-enhanced recombinant AAV particle (e.g., see above). In some cases, the contacting occurs such that said contacting results in retrograde transport of at least some of the viral particle's contents. In some cases, the contacting occurs in an individual's spinal cord, e.g., the neuron can be a neuron spinal cord projecting neuron such as a neuron of the rostral ventromedial medulla (RVM) or a neuron of the locus coeruleus (LC). In some cases, the contacting occurs in an individual's thalamus, e.g., the neuron can be corticothalamic projecting neurons. In some cases, the neuron is an opioid receptor mu 1 (OPRM1) expressing neuron.

Reagents, compositions, and kits/systems that find use in practicing the subject methods are provided.

### BRIEF DESCRIPTION OF THE DRAWINGS

The following detailed description of embodiments of the invention will be better understood when read in conjunction with the appended drawings. It should be understood that the invention is not limited to the precise arrangements and instrumentalities of the embodiments shown in the drawings.

FIG. 1 (panels a-i). Labeling and recording of OPRM1⁺ RVM^{SC} neurons. (panel a) Design of OPRM1-Cre knockin mice. (panel b) Schematic shows spinal injection of AAV8-retro-H2BClover3-FLEX(LoxP)-H2BRuby3 at P1.5 in OPRM1-Cre mice. (panel c) 3D reconstruction of brainstem shows retrogradely labeled OPRM1⁺ (yellow) and OPRM1⁻ (green) RVM^{SC} neurons. Scale Bar: 1 mm. (panel d) Distribution of RVM^{SC} neurons along A-P axis. (panel e) Example images and quantification of molecular characterization of OPRM1⁺ RVM^{SC} neurons (n=8 slides from 4 mice). OPRM1⁺ RVM^{SC} neurons were retrogradely labeled with spinal injection of AAV8-retro-FLEX(LoxP)-Rpl22-3XHA. RNAscope probes were used to visualize OPRM1 (magenta), vGAT (red) and TPH2 (cyan). HA tag (green) was visualized by immunostaining. Scale Bar: 50 μm. (panel f) Experimental timeline for panel g. W, week. Representative image shows cannula track and jGCaMP7s expression. Scale Bar: 500 m. (panels g and h) Example calcium traces (g) and quantification (h) of OPRM1⁺ RVM^{SC} neurons respond to mechanical (Von Frey) and thermal (Hargreaves and Plantar cold) stimuli in normal and SNI mice. Single trial traces are in black and mean of averaged traces are in Red (n=4-5). Black arrow indicates time of paw withdrawal caused by stimuli. Red arrow indicates time of spontaneous paw withdrawal. Scale Bar: 2 s, 0.5% ΔF/F for single trial trace from normal mouse; 2 s, 2% ΔF/F for single trial trace from SNI mouse; 5 s, 0.5% ΔF/F for averaged traces. Mann-Whitney test, * P<0.05, ** P<0.01. (panel i) Quantification of calcium responses in OPRM1⁺ RVM^{SC} neurons to mechanical stimuli before SNI and 2, 7, 14, 28 days after SNI (n=3). Mean±SEM

FIG. 2 (panels a-h). OPRM1⁺ RVM^{SC} neurons are required for initiation and maintenance of pain sensitization. (panel a) Experimental timeline for schematic for subsequent panels. W, week. (panel b) Quantification of saline (black) or clozapine on mechanical threshold (left) and thermal withdrawal latency (right) of hM4Di (blue, n=8) or hM3Dq (red, n=10) expressing mice. Wilcoxon matched-pairs signed rank test, ** P<0.01. (panel c) Representative images of caspase caused ablation of OPRM⁺ RVM^{SC} neurons (see methods). OPRM1⁺ RVM neurons are labeled with Ruby3 (red). OPRM1⁺ RVM^{SC} neurons express both Ruby3 and Clover3 (yellow). Scale Bar: 100 μm. (panel d) Quantification of mechanical thresholds of control- (black, n=6) and Caspase3- (orange, n=7) expressing mice after SNI (left). Representative image shows OPRM⁺ RVM^{SC} neurons terminals (yellow) and mechanical stimuli evoked c-Fos signals (white) in control- (upper panel) but not Caspase3-(lower panel) expressing mice (right). c-Fos signals are in white. Scale Bar: 100 μm. Mann-Whitney test, *** P<0.001. (panels e and f) Quantification of mechanical thresholds (e) and thermal withdrawal latency (f) of control- (black, n=4) and Caspase3- (orange, n=4) expressing mice after CFA injection. Mann-Whitney test, * P<0.05. (panel g) Quantification of mechanical thresholds after saline (black, n=8) or clozapine (blue, n=8) infusion in hM4Di expressing mice after SNI. Wilcoxon matched-pairs signed rank test, ** P<0.01. (panel h) Example traces (upper panel) and quantification (lower panel) of mechanical stimuli (0.16 g Von Frey filament) evoked CPA in control (black, n=5) but not in hM4Di (blue, n=5) expressing mice after SNI. Mann-Whitney test, * P<0.05. Mean±SEM.

FIG. 3 (panels a-i). CaMKv in OPRM1⁺ RVM^{SC} neurons drives persistent pain. (panel a) Experimental timeline (left) and quantification (right) of Ribotag sequencing of OPRM1V RVM^{SC} neurons from SNI (n=12, 3 groups 4 mice per group) or sham surgery (n=12, 3 groups 4 mice per group) mice. (panel b) Representative images of knocking down CaMKv using CaMKv-shmiR. OPRM⁺ RVM^{SC} neurons are visualized in green and immunostaining of CaMKv in magenta. Scale Bar: 50 μm. (panel c) Experimental timeline for panels d and e. (panel d) Quantification of mechanical threshold of control-shmiR (black, n=5) and CaMKv-shmiR (blue, n=6) expressing SNI mice. Mann-Whitney test, * P<0.05, ** P<0.01. (panel e) Quantification of mechanical threshold of control-shmiR (black, n=4) and CaMKv-shmiR (blue, n=4) expressing CFA mice. Mann-Whitney test, * P<0.05. (panel f) Experimental timeline (left) and quantification of mechanical threshold (right) of control-shmiR (black, n=5) and CaMKv-shmiR (blue, n=7) expressing mice after SNI surgery. Mann-Whitney test, ** P<0.01. (panel g) Experimental timeline for h. (panel h) Quantification of mechanical threshold (left) and thermal withdrawal latency (right) of mice overexpressed CaMKv in OPRM1⁺ RVM^{SC} neurons (n=7). Dunn's multiple comparisons test, * P<0.05, ** P<0.01. (panel i) Example traces (left) and quantification (right) of mechanical stimuli (0.16 g Von Frey filament) evoked CPA in control (black, n=6) and CaMKv (red, n=5) overexpressing mice. Mann-Whitney test, * P<0.05. Mean±SEM.

FIG. 4 (panels a-i) Lateral SC inputs onto OPRM1⁺ RVM^{SC} neurons drives persistent pain. (panel a) Schematic (upper panel) of pharmacogenetic silencing of the PAG-RVM pathway. Representative image shows vGlut2⁺ terminals (red) from PAG in the RVM. Scale Bar: 500 μm. (panel b) Quantification of mechanical thresholds of saline (black) and clozapine (blue) infusion in the same group of hM4Di expressing mice (n=8). (panels c, d, and e) Experimental timeline (c), representative images (d), and quantification (e) of monosynaptic inputs onto OPRM1⁺ RVM^{SC} neurons (n=6). Scale Bar: 250 m. Inset, Representative images (upper) and quantification (lower) of RNAscope staining of vGlut2 (green) and vGAT (blue) expression in mCherry positive retrogradely labeled input neurons (n=3). Scale Bar: 50 μm. (panel f) Experimental timeline for panels g and h. (panel g) Quantification of mechanical thresholds of BFP- (black, n=5) and Caspase3- (orange, n=5) expressing mice after SNI. Mann-Whitney test, ** P<0.01. (panel h) Quantification of CaMKv (upper panel) and CaMK2a expression in OPRM1⁺ RVM^{SC} neurons from control (white, normalized to 100%,n=4), SNI (grey, n=4), and SNI+ablation of lSC inputs (orange, n=4). Dunn's multiple comparisons test, * P<0.05. (panel i) Quantification of mechanical thresholds of saline (black, n=6) and clozapine (blue, n=5) infusion in the same group of hM4Di expressing mice. Wilcoxon matched-pairs signed rank test, * P<0.05. Mean±SEM.

FIG. 5 (panels a-c) Generation and characterization of OPRM1-Cre knockin mouse line. (panel a) Gene targeting strategy used to generate OPRM1-Cre knockin mouse line. (panel b) Morphine produced similar analgesic effect in WT and OPRM1^{Cre/+}, but not in OPRM1^{Cre/Cre} mice (WT saline vs. WT 5 mg Kg⁻¹ morphine, P=0.0089, Oprm1^{Cre/+} saline vs. Oprm1^{Cre/+} 5 mg Kg⁻¹ morphine, P=0.0053, Oprm1^{Cre/Cre} saline vs. Oprm1^{Cre/Cre} 5 mg Kg⁻¹ morphine, P>0.9999, Dunn's multiple comparisons test, 5 mice for each genotype). (panel c) Morphine increased locomotion in in WT and OPRM1^{Cre/+}, but not in OPRM1^{Cre/Cre} mice (WT saline vs. WT 15 mg Kg⁻¹ morphine, 6 mice P=0.0313, Oprm1^{Cre/+} saline vs. Oprm1^{Cre/+} 15 mg Kg⁻¹ morphine, 6 mice, P=0.0313, Oprm1^{Cre/Cre} saline vs. Oprm1^{Cre/Cre} 15 mg Kg⁻¹ morphine, 5 mice, P=0.0626, Wilcoxon matched-pairs signed rank test).

FIG. 6 (panels a-d) OPRM1⁺ ascending neurons in the RVM. (panel a) Schematic shows spinal injection of AAV8-retro-FLEX(LoxP)-Flp at P1.5 in OPRM1-Cre mice, then four weeks later, RVM injection of AAV8-FLEX(LoxP)-Ruby3-FLEX(FRT)-Clover3. This intersectional strategy leads to the expression of Ruby3 in all OPRM1⁺ neurons in the RVM and co-expression of both Ruby3 and Clover3 in the descending OPRM1⁺ RVM neurons. (panel b) Representative image shows co-expression of both Ruby3 and Clover3 in the terminals of descending OPRM1⁺ RVM^{SC} neurons in the spinal cord. Scale Bar: 200 μm. (panel c) Representative image shows the Ruby3 expressing terminal of OPRM1V ascending RVM neurons in the thalamus. Notably, OPRM1⁺ RVM^{SC} neurons do not collateralize to the thalamus. Scale Bar: 1 mm. (panel d) Representative image shows OPRM1⁺ LC^{SC} neurons labeled by intraspinal injection of AAV8-retro-FLEX(LoxP)-Rpl22-3XHA. RNAscope probes were used to visualize OPRM (red), HA tag (green) was visualized by immunostaining. Scale Bar: 100 μm

FIG. 7 (panels a-e) Developing and characterization of AAV8-retro. (panel a) Images of dissected spinal cord from mice injected with 3 μl (left) and 1 μl (right) of AAV8-retro-mCherry. Neonatal injection of 1 μl or 3 μl of AAV8-retro-mCherry led the virus infection of the entire lumbar region or both lumber and thoracic regions of the spinal cord, respectively. Inset shows the expression of mCherry was restricted largely in the dorsal horn. Scale Bar: 1 mm. (panel b) Plasmid map shows the site for inserting the decapeptide LADQDYTKTA peptides between N590 and T591 of the AAV8 capsid (AAV8-retro). (panel c) Schematic shows co-injection of AAV8-retro-mCherry and AAV2-retro-eGFP into the spinal cord of P1.5 wild type mice. (panel d) Representative images of retrogradely labeled RVM neurons by AAV8-retro-mCherry (red), AAV2-retro-eGFP (green), and merge (yellow). Scale Bar: 200 μm. (panel e) Quantification of d (n=3).

FIG. 8 (panels a-b) Representative images of knocking down CaMK2a using CaMK2a-shmiR. (panel a) OPRM1⁺ RVM^{SC} neurons are visualized in green and immunostaining of CaMK2a in magenta. Scale Bar: 50 μm. (panel b) Quantification of mechanical threshold of control-shmiR (black, n=5, same data as FIG. 3b) and CaMK2a-shmiR (red, n=3) expressing SNI mice.

FIG. 9 Representative AAV serotypes classified into clades (See, e.g., Gao et. al., J Virol. 2004 June; 78(12): 6381-6388; and U.S. Pat. No. 7,906,111). AAV8 is a member of Clade E.

FIG. 10A-10I Amino acid sequences of the capsid protein of representative clade E AAVs (See, e.g., Gao et. al., J Virol. 2004 June; 78(12): 6381-6388; and U.S. Pat. No. 7,906,111) (also see FIG. 9).

FIG. 11 Amino acid sequence alignment of an insert region (boxed) of representative clade E capsid proteins. In this example, the 10-amino acid retro-sequence can be inserted in the middle of the boxed amnio acids. For example, ‘AAB8-retro’ (SEQ ID NO: 32) was produced by inserting the retro-sequence LADQDYTKTA (SEQ ID NO: 1) in the middle of the boxed amino acids of AAV8 (SEQ ID NO: 2) (also see FIG. 7 panel b). Insertion of SEQ ID NO: 1 into SEQ ID Nos: 2-27 as depicted generates SEQ ID Nos: 32-57, respectively.

FIG. 12 One example of a DNA encoding an shRNA used to target CamKv. This shRNA was used in the working examples described herein. The shRNA depicted in this particular embodiment is embedded in a miR-155 backbone.

FIG. 13 AAV2-retro-Cre and AAV8-retro-Cre were injected into the thalamus of a Ai9 reporter mice to retrogradely label corticothalamic projection neurons. Same titer AAV8-retro-Cre retrogradely labeled 5 times more neurons than AAV2-retro-Cre. (Blue are pan neuronal marker Neun, red are RFP signal for retrogradely labeled neurons).

FIG. 14 (panels a-f). Efficacy and safety of using an RNAi agent (in this embodiment: AAV8-retro-CaMKv-shmiR via intraspinal injection) to treat neuropathic pain. (panel a), Experimental timeline for b. (panel b), Quantification of mechanical thresholds in mice injected with 3.0E9 vg (black, n=6) and 3.8E11vg (blue, male, n=5; red, female, n=4) of AAV8-retro-CaMKv-shmiR after SNI-surgery. Mann-Whitney test, * P<0.05. (panel c), Representative images of AAV8-retro-CaMKv-shmiR injected neurons in the spinal cord (upper panel) and RVM (lower panel). Scale bar: 200 μm. (panel d), Quantification of mechanical (left) and thermal thresholds (right) in wide type (WT, black, n=7) and AAV8-retro-CaMKv-shmiR expressing (blue, n=7) non-injured mice. (panel e), Quantification of total travel distance of wide type (WT, black, n=6) and AAV8-retro-CaMKv-shmiR expressing (blue, n=7) non-injured mice in an open field. (panel f), Quantification of object exploration time (left) and preference index (right) of wide type (WT, black, n=6) and AAV8-retro-CaMKv-shmiR expressing (blue, n=6) non-injured mice in novel object recognition test. Mean±SEM.

FIG. 15 (panels a-c) Intraspinal injection of AAV8-retro-CaKMv-shmiR did not cause inflammation. (panel a), Representative images of retrogradely labeled neurons (green) at somatosensory cortex (S1, left), paraventricular hypothalamus (PVH, middle) and dorsal root ganglia (DRG, right). n=6, Scale bar: 1 mm; 500 μm; 100 μm. (panel b), Representative image of immunostaining of CD3 (red) in the spinal cord (left) and spleen (right) after intraspinal injection of AAV8-retro-CaKMv-shmiR. No detectable CD3 at the injection site in the spinal cord. n=3, Scale bar: 1 mm; 500 μm. (panel c), Representative image of immunostaining of iba-1 (red) in the spinal cord from mice without (left) and with (right) intraspinal injection of AAV8-retro-CaKMv-shmiR. AAV injection does not increases iba-1 expression. n=3, Scale bar: 500 μm.

### DETAILED DESCRIPTION

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Certain ranges and/or values are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited. It is understood that the present disclosure supersedes any disclosure of an incorporated publication to the extent there is a contradiction.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that as used herein and in the appended claims, the singular forms “a,” “an,” and “the” include plural referents unless the context clearly dictates otherwise. As such, the articles “a” and “an” are used herein to refer to one or to more than one (i.e., to at least one) of the grammatical object of the article. By way of example, “an element” means one element or more than one element. Thus, for example, reference to “a cell” includes a plurality of such cells and reference to “the polypeptide” includes reference to one or more polypeptides and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is also to be understood that the terminology used herein is for the purpose of describing particular embodiments only, and is not intended to be limiting.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible. For example, it is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

While the apparatus and method has or will be described for the sake of grammatical fluidity with functional explanations, it is to be expressly understood that the claims, unless expressly formulated under 35 U.S.C. § 112, are not to be construed as necessarily limited in any way by the construction of “means” or “steps” limitations, but are to be accorded the full scope of the meaning and equivalents of the definition provided by the claims under the judicial doctrine of equivalents, and in the case where the claims are expressly formulated under 35 U.S.C. § 112 are to be accorded full statutory equivalents under 35 U.S.C. § 112.

As noted above, provided are methods of treating an individual in need (e.g., administering to an individual who has chronic pain a therapy that reduces CamKv activity in OPRM1 expressing neurons of the RVM), retrograde-enhanced clade E variant adeno-associated virus (AAV) capsid proteins, transduction systems that include nucleic acids that encode such capsid proteins, AAV viral particles that include such capsid proteins, methods of making viral particles that include such capsid proteins, and methods of expressing transgenes of interest using viral particles that include such capsid proteins.

### Methods of Treatment

The present disclosure provides methods treatment as well as methods of expressing a transgene in a neuron—in some cases to a neuron of an individual. In some cases, the neuron is in vivo, e.g., is in the body of an individual. In some cases, a method of treatment, e.g., to treat chronic pain, does not include the use of a subject retrograde-enhanced clade E variant AAV (having a retrograde-enhanced clade E variant AAV capsid protein) as described herein. In other cases, a method of treatment, e.g., to treat chronic pain, does include the use of a subject retrograde-enhanced clade E variant AAV (having a retrograde-enhanced clade E variant AAV capsid protein) as described herein—and therefore a subject method of treatment can in some cases include a method of expressing a transgene in a neuron (described in more detail elsewhere herein).

In some cases, the individual to be treated has chronic pain, and the subject method is a way to treat/combat/alleviate the chronic pain. It is to be understood that when referring to “chronic pain” there are two generally accepted types of chronic pain: “inflammatory nociceptive pain” (also referred to herein as “inflammatory pain”) and “neuropathic pain”. Inflammatory nociceptive pain is associated with tissue damage and the resulting inflammatory process. It is adaptive in that it elicits physiologic responses that promote healing. Neuropathic pain is produced by damage to the neurons (e.g., in the peripheral and/or central nervous systems) and involves sensitization of these systems. In peripheral sensitization, there is an increase in the stimulation of peripheral nociceptors that amplifies pain signals to the central nervous system. In central sensitization, neurons that originate in the dorsal horn of the spinal cord become hyperstimulated, increasing pain signals to the brain and thereby increasing pain sensation. Chronic pain may involve a mix of both inflammatory and neuropathic components. For example, in inflammatory nocicpetive pain, inflammation may cause damage to the neurons and produce neuropathic pain. Likewise, neuronal injury may cause an inflammatory reaction (neurogenic inflammation) that contributes to inflammatory pain.

As noted above, the inventors have discovered the CamKv activity in OPRM1 expressing neurons of the RVM drives chronic pain. Thus, the treatment methods disclosed herein can be used to reduce CamKv activity in OPRM1 expressing neurons of the RVM. As such, in some embodiments, a subject treatment method includes a step of administering to an individual who has chronic pain a therapy that reduces CamKv activity in opioid receptor mu 1 (OPRM1) expressing neurons of the individual's rostral ventromedial medulla (RVM).

Reduction of CamKv protein activity in the RVM can be accomplished in a variety of different ways, and as demonstrated by the working examples below, any one of these ways can be useful to successfully treat chronic pain. For example, in some cases a subject method includes reducing excitatory input into the RVM from RVM-projecting lateral superior colliculus (lSCIndG) neurons. In some cases, reduction of CamKv protein activity in the RVM can be accomplished by increasing (stimulating) inhibitory input into the RVM, which can in some cases be accomplished by stimulating inhibitory input into the RVM from neurons of the zona incerta. Thus, in some cases, reduction of CamKv protein activity in the RVM can be accomplished by deep brain stimulation of zona incerta neurons, thus stimulating inhibitory input into the RVM.

In some embodiments, reduction of CamKv protein activity in the RVM can be accomplished by administering an agent that reduces CamKv activity by directly binding to CamKv and blocking its function or destabilizes CamKv—e.g., targeting it for destruction. For example, in some cases reduction of CamKv protein activity in the RVM can be accomplished by providing an agent (such as a small molecule or a protein such as an antibody) that targets CamKv and blocks its function.

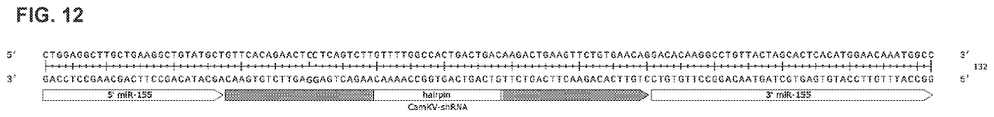

In some embodiments, reduction of CamKv protein activity in the RVM can be accomplished by administering an agent that reduces production (expression, and thereby protein levels) of CamKv protein (e.g., by blocking translation or by reducing the amount of mRNA present). This can be accomplished at the RNA level by translation blockers, RNAs such as antisense RNAs, ribozymes, an RNAi agent, and the like. As such, in some cases a subject method includes administration of an RNAi agent that targets CamKv. The term “RNAi agent” is used herein to mean any agent that can be used to induce a gene specific RNA interference (RNAi) response in a cell. Suitable examples of RNAi agents include, but are not limited to short interfering RNAs (siRNAs), short hairpin RNAs (shRNAs), and micro RNAs (miRNA). An RNAi agent (e.g., shRNA, siRNA, miRNA) that targets CamKv is an agent that targets the mRNA encoding the CamKv protein. RNAi agents can readily be designed to specifically target any desired mRNA (e.g., one encoding CamKv) by choosing an appropriate nucleotide sequence. In some cases, a subject RNAi agent is an shRNA that targets CamKv and is embedded in a microRNA backbone such as a miR-155 backbone. In some cases, the shRNA-encoding sequence includes the sequence depicted in FIG. 12 (which includes a CamKv targeting sequence embedded in a miR-155 backbone). In some cases, the shRNA-encoding sequence includes a sequence that has 90% or more (e.g., 95% or more, 97% or more, 98% or more, 99% or more, or 99.5% or more) sequence identity with the sequence depicted in FIG. 12 (which includes a CamKv targeting sequence embedded in a miR-155 backbone). In some cases, the shRNA-encoding sequence includes the CamKv-shRNA portion of the sequence depicted in FIG. 12. In some cases, the shRNA-encoding sequence includes a sequence that has 90% or more (e.g., 95% or more, 97% or more, 98% or more, 99% or more, or 99.5% or more) sequence identity with the CamKv-shRNA portion of the sequence depicted in FIG. 12. In some cases, the shRNA-encoding sequence targets the same target sequence that the shRNA of FIG. 12 targets. In some cases, a subject shRNA will include one or more mismatches in the stem loop, and in some cases the stem loop will not have a mismatch (i.e., will be a perfect duplex).

Various RNAi agent designs (RNAi agents with various features) are known in the art and any convenient RNAi agent (e.g., one that targets CamKv) can be used. For example, various designs of RNAi agents (as well as methods of their delivery) can be found in numerous patents, including, but not limited to U.S. Pat. Nos. 7,022,828; 7,176,304; 7,592,324; 7,667,028; 7,718,625; 7,732,593; 7,772,203; 7,781,414; 7,807,650; 7,879,813; 7,892,793; 7,910,722; 7,947,658; 7,973,019; 7,973,155; 7,981,446; 7,993,925; 8,008,271; 8,008,468; 8,017,759; 8,034,922; 8,399,653; 8,415,319; 8,426,675; 8,466,274; 8,524,679; 8,524,679; 8,569,065; 8,569,256; 8,569,258; 9,233,102; 9,233,170; and 9,233,174; all of which are incorporated herein by reference.

Any convenient version of RNAi agent can be used. For example, endogenous miRNA sequences can be used as a scaffold for artificial miRNA. For example, in some cases an shRNA of interested can be embedded in a miRNA backbone such as a mir-155 backbone (see, e.g., Fowler et al., Nucleic Acids Res. 2016 Mar. 18; 44(5): e48, “Improved knockdown from artificial microRNAs in an enhanced miR-155 backbone: a designer's guide to potent multi-target RNAi”; and Uva et al., RNA. 2013 March; 19(3):365-79, “Rat mir-155 generated from the lncRNA Bic is ‘hidden’ in the alternate genomic assembly and reveals the existence of novel mammalian miRNAs and clusters”—both of which references are hereby incorporated by reference in their entirety). See the working examples below for such an example that was used for targeting CamKv (referred to below as a CaMKv shmiR, or “CaMKv-shmiR”).

Another way to reduce production (expression, and thereby protein levels) of CamKv protein is to provide an agent that reduces transcription of CamKv-encoding mRNA. Such agents include, for example, genome-targeting proteins such as Zinc Finger proteins, TALEs, and CRISPR/Cas effector proteins (such as Cas9, Cas12, and the like) that reduce transcription. In some cases, the genome-targeting proteins can be nuclease inactivated, but associates with (e.g., can be fused to), a domain that represses transcription (e.g., a transcriptional repressor, a chromatin modifier, a DNA methylase, etc.). In the case of CRISPR/CAS effector proteins, such a domain can be fused to the CRISPR/Cas effector protein, or can be a fusion protein that binds to a sequence on the guide RNA (see, e.g., MS2-fusion proteins).

For any of the above treatments that include providing an agent to the individual, the agent can be delivered systemically (e.g., intravenous), locally (e.g., local injection), or by any route, for example, by injection, infusion, orally (e.g., ingestion or inhalation), or topically (e.g., transdermally). Possible delivery and administration methods can include parenteral, intravenous, intramuscular, intraperitoneal, intradermal, subcutaneous, intracavity, intracranial, transdermal (topical), transmucosal and rectal administration. Example administration and delivery routes include intravenous, intraperitoneal, intrarterial, parenteral, subcutaneous, intra-pleural, topical, dermal, intradermal, transdermal, transmucosal, oral (alimentary), mucosal, respiration, intranasal, intubation, intrapulmonary, intrapulmonary instillation, buccal, sublingual, intravascular, intrathecal, intracavity, iontophoretic, intraocular, ophthalmic, optical, intraglandular, intraorgan, and intralymphatic. In some cases the delivery route is systemic (e.g., parenteral, intravenous).

For any of the above treatments that include providing an agent to the individual, in some cases the method can employ a subject retrograde-enhanced clade E variant AAV (an AAV that includes a subject retrograde-enhanced clade E variant AAV capsid protein—which is described in detail below) to deliver the agent to the RVM neurons in a retrograde fashion. As such, in some cases a subject retrograde-enhanced clade E variant AAV that includes a nucleic acid with a transgene sequence is administered to an individual (e.g., one with chronic pain), where the transgene sequence is useful for targeting CamKv. As an example, in some cases the transgene sequence encodes an RNAi agent such as an shRNA (e.g., an shRNA embedded in a miRNA backbone) that targets CamKv. In some cases, a subject RNAi agent is an shRNA that targets CamKv and is embedded in a microRNA backbone such as a miR-155 backbone. In some cases, the shRNA-encoding sequence includes the sequence depicted in FIG. 12 (which includes a CamKv targeting sequence embedded in a miR-155 backbone). In some cases, the shRNA-encoding sequence includes a sequence that has 90% or more (e.g., 95% or more, 97% or more, 98% or more, 99% or more, or 99.5% or more) sequence identity with the sequence depicted in FIG. 12 (which includes a CamKv targeting sequence embedded in a miR-155 backbone). In some cases, the shRNA-encoding sequence includes the CamKv-shRNA portion of the sequence depicted in FIG. 12. In some cases, the shRNA-encoding sequence includes a sequence that has 90% or more (e.g., 95% or more, 97% or more, 98% or more, 99% or more, or 99.5% or more) sequence identity with the CamKv-shRNA portion of the sequence depicted in FIG. 12. In some cases, the shRNA-encoding sequence targets the same target sequence that the shRNA of FIG. 12 targets. In some cases, a subject shRNA will include one or more mismatches in the stem loop, and in some cases the stem loop will not have a mismatch (i.e., will be a perfect duplex).

As another example, in some cases the transgene sequence encodes a CRISPR/Cas guide RNA which can be used to target a CRISPR/Cas effector protein (e.g., one associated with a transcriptional repressor as discussed above) to the CamKv locus to block transcription of CamKv mRNA. In some cases the transgene sequence encodes the CRISPR/Cas effector protein. In some cases, the variant AAV includes a nucleic acid that encodes both a CRISPR/Cas effector protein and a CRISPR/Cas guide RNA. As discussed in more detail elsewhere herein, the transgene sequence (or an expression cassette that includes the transgene sequence) can be flanked by ITRs.

### Co-Administration

In some cases any of the above treatments (e.g., stimulating inhibitory input into the RVM, providing an agent such as an RNAi agent that targets CamKv, providing an agent that targets CamKv protein, providing a retrograde-enhanced clade E variant AAV) can be administered with an additional therapy or agent. Examples of such additional therapies or agents include (but are not limited to), in any combination: drug therapy (e.g., acetaminophen, a nonsteroidal anti-inflammatory drug (NSAID) such as aspirin, ibuprofen, celecoxib, or naproxen, a topic pain reliever, an anti-anxiety drug such as diazepam, an antidepressant such as duloxetine, a painkiller such as codeine, fentanyl, oxycodone or oxycodone and acetaminophen, hydrocodone or hydrocodone and acetaminophen, morphine, or codeine, cannabis, a steroid such as a local steroid injection, an epidural, an anticonvulsant, and the like), acupuncture, acupressure, physical therapy, cognitive therapy, behavioral therapy, exercise, relaxation techniques such as meditation, massage, or yoga, psychological counseling, a surgical implant, transcutaneous electrical nerve stimulation (TENS).

The terms “co-administration”, “co-administer”, and “in combination with” include the administration of two or more therapies either simultaneously, concurrently or sequentially within no specific time limits. In some embodiments, agents are present in the cell or in the subject's body at the same time or exert their biological or therapeutic effect at the same time. In some embodiments, therapeutic agents are in the same composition or unit dosage form. In other embodiments, therapeutic agents are in separate compositions or unit dosage forms. In certain embodiments, a first therapy (e.g., agent) can be administered prior to (e.g., minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapy (e.g., agent).

In some cases, an agent (e.g., an RNAi that targets CamKv, a subject retrograde-enhanced clade E variant AAV (e.g., formulated as a pharmaceutical composition) is co-administered with an agent or therapy such as those listed above that can be used to treat chronic pain. Such administration may involve concurrent (i.e. at the same time), prior, or subsequent administration of the agent and/or therapy with respect to the administration of an agent or agents of the disclosure. A person of ordinary skill in the art would have no difficulty determining the appropriate timing, sequence and dosages of administration for particular drugs and compositions of the present disclosure.

### Retrograde-Enhanced Clade E Variant AAV Capsid Proteins

Retrograde transport shuttles molecules and/or organelles away from axon termini toward the cell body. As used herein, “retrograde” transport refers to movement in an axon toward its cell body. A retrograde-enhanced clade E variant AAV capsid protein provides for an enhanced ability for an AAV viral particle that includes such a capsid protein to transduce neurons in a retrograde fashion. An AAV particle (virion) that includes a subject retrograde-enhanced clade E variant AAV capsid protein is referred to herein as a retrograde-enhanced clade E variant AAV. In addition to its ability to infect cells such as neurons at the site of exposure such as neuronal cell body, a subject retrograde-enhanced clade E variant AAV can access neuronal cell bodies by contact at the axons. As such, a subject retrograde-enhanced clade E variant AAV capsid protein provides retrograde access to projection neurons.

The term “enhanced” in the context of “retrograde-enhanced” is used herein to refer to an enhanced ability (any degree of enhancement can be acceptable) for a subject clade E variant AAV viral particle (a viral particle having a subject clade E variant AAV capsid protein such as AAV8-retro) to transduce neurons in a retrograde fashion relative to AAV8 (a viral particle having an AAV8 capsid protein—see, e.g., the AAV8 capsid protein set for as SEQ ID NO. 2), unless a different comparator is expressly stated. In some cases, e.g., when expressly stated, the comparison can be to AAV2 (a viral particle having an AAV2 capsid protein—see, e.g., the AAV2 capsid protein set for as SEQ ID NO. 64). In some cases, e.g., when expressly stated, the comparison can be to AAV2-retro (a viral particle having an AAV2-retro capsid protein—see, e.g., the AAV2-retro capsid protein set for as SEQ ID NO. 65).

In some cases, e.g., when expressly stated, the comparison can be to the corresponding wild type AAV (a viral particle having the corresponding wild type capsid). To illustrate, if the retrograde-enhanced clade E variant AAV capsid protein is rh10-retro, then the corresponding wild type AAV would be rh10 (a viral particle having an rh10 AAV capsid protein), if the retrograde-enhanced clade E variant AAV capsid protein is hul7-retro, then the corresponding wild type AAV would be hul7 (a viral particle having an rh10 AAV capsid protein), if the retrograde-enhanced clade E variant AAV capsid protein is AAV8-retro, then the corresponding wild type AAV would be AAV8 (a viral particle having an rh10 AAV capsid protein), and the like.

In some cases, the efficiency of retrograde transduction for an AAV that includes a subject retrograde-enhanced clade E variant AAV capsid protein (e.g., AAV8-retro or rh10-retro) relative to the efficiency of retrograde transduction for AAV8 is 1.2-fold or more (e.g., 1.5-fold or more, 2-fold or more, 2.5-fold or more, 3-fold or more, 3.5-fold or more, 4-fold or more, or 5-fold or more). This can be measured by any convenient assay, e.g., one that compares the number of neurons that are retrograde transduced by a virus having a retrograde-enhanced clade E variant AAV capsid protein to the number of neurons that are retrograde transduced by a control virus (which can be AAV8, but a number of different control viruses can be suitable as controls—depending on the desired comparison, as discussed herein). In some cases, the efficiency of retrograde transduction for an AAV that includes a subject retrograde-enhanced clade E variant AAV capsid protein (e.g., AAV8-retro or rh10-retro) relative to the efficiency of retrograde transduction for AAV8 is 2-fold or more (e.g., 2.5-fold or more, 3-fold or more, 3.5-fold or more, 4-fold or more, or 5-fold or more).

In some cases, the efficiency of retrograde transduction for an AAV that includes a subject retrograde-enhanced clade E variant AAV capsid protein (e.g., AAV8-retro or rh10-retro) relative to the efficiency of retrograde transduction for AAV2 (a viral particle having an AAV2 capsid protein) is 1.2-fold or more (e.g., 1.5-fold or more, 2-fold or more, 2.5-fold or more, 3-fold or more, 3.5-fold or more, 4-fold or more, or 5-fold or more). In some cases, the efficiency of retrograde transduction for an AAV that includes a subject retrograde-enhanced clade E variant AAV capsid protein (e.g., AAV8-retro or rh10-retro) relative to the efficiency of retrograde transduction for AAV2 is 2-fold or more (e.g., 2.5-fold or more, 3-fold or more, 3.5-fold or more, 4-fold or more, or 5-fold or more).

In some cases, the efficiency of retrograde transduction for an AAV that includes a subject retrograde-enhanced clade E variant AAV capsid protein (e.g., AAV8-retro or rh10-retro) relative to the efficiency of retrograde transduction for AAV2-retro (a viral particle having an AAV2-retro capsid protein) is 1.2-fold or more (e.g., 1.5-fold or more, 2-fold or more, 2.5-fold or more, 3-fold or more, 3.5-fold or more, 4-fold or more, or 5-fold or more). In some cases, the efficiency of retrograde transduction for an AAV that includes a subject retrograde-enhanced clade E variant AAV capsid protein (e.g., AAV8-retro or rh10-retro) relative to the efficiency of retrograde transduction for AAV2-retro is 2-fold or more (e.g., 2.5-fold or more, 3-fold or more, 3.5-fold or more, 4-fold or more, or 5-fold or more).

In some cases, the efficiency of retrograde transduction for an AAV that includes a subject retrograde-enhanced clade E variant AAV capsid protein (e.g., AAV8-retro or rh10-retro) relative to the efficiency of retrograde transduction for a corresponding wild type AAV (a viral particle having a corresponding wild type AAV capsid protein) is 1.2-fold or more (e.g., 1.5-fold or more, 2-fold or more, 2.5-fold or more, 3-fold or more, 3.5-fold or more, 4-fold or more, or 5-fold or more). In some cases, the efficiency of retrograde transduction for an AAV that includes a subject retrograde-enhanced clade E variant AAV capsid protein (e.g., AAV8-retro or rh10-retro) relative to the efficiency of retrograde transduction for a corresponding wild type AAV (a viral particle having a corresponding wild type AAV capsid protein) is 2-fold or more (e.g., 2.5-fold or more, 3-old or more, 3.5-fold or more, 4-fold or more, or 5-fold or more).

The following are examples of capsid proteins (see also Table 1):

AAV8
(SEQ ID NO: 2)
MAADGYLPDWLEDNLSEGIREWWALKPGAPKPKANQQKQDDGRGL
VLPGYKYLGPFNGLDKGEPVNAADAAALEHDKAYDQQLQAGDNPY
LRYNHADAEFQERLQEDTSFGGNLGRAVFQAKKRVLEPLGLVEEG
AKTAPGKKRPVEPSPQRSPDSSTGIGKKGQQPARKRLNFGQTGDS
ESVPDPQPLGEPPAAPSGVGPNTMAAGGGAPMADNNEGADGVGSS
SGNWHCDSTWLGDRVITTSTRTWALPTYNNHLYKQISNGTSGGAT
NDNTYFGYSTPWGYFDFNRFHCHFSPRDWQRLINNNWGFRPKRLS
FKLFNIQVKEVTQNEGTKTIANNLTSTIQVFTDSEYQLPYVLGSA
HQGCLPPFPADVFMIPQYGYLTLNNGSQAVGRSSFYCLEYFPSQM
LRTGNNFQFTYTFEDVPFHSSYAHSQSLDRLMNPLIDQYLYYLSR
TQTTGGTANTQTLGFSQGGPNTMANQAKNWLPGPCYRQQRVSTTT
GQNNNSNFAWTAGTKYHLNGRNSLANPGIAMATHKDDEERFFPSN
GILIFGKQNAARDNADYSDVMLTSEEEIKTTNPVATEEYGIVADN
LQQQNTAPQIGTVNSQGALPGMVWQNRDVYLQGPIWAKIPHTDGN
FHPSPLMGGFGLKHPPPQILIKNTPVPADPPTTFNQSKLNSFITQ
YSTGQVSVEIEWELQKENSKRWNPEIQYTSNYYKSTSVDFAVNTE
GVYSEPRPIGTRYLTRNL
AAV8-retro
(SEQ ID NO: 32)
MAADGYLPDWLEDNLSEGIREWWALKPGAPKPKANQQKQDDGRGL
VLPGYKYLGPFNGLDKGEPVNAADAAALEHDKAYDQQLQAGDNPY
LRYNHADAEFQERLQEDTSFGGNLGRAVFQAKKRVLEPLGLVEEG
AKTAPGKKRPVEPSPQRSPDSSTGIGKKGQQPARKRLNFGQTGDS
ESVPDPQPLGEPPAAPSGVGPNTMAAGGGAPMADNNEGADGVGSS
SGNWHCDSTWLGDRVITTSTRTWALPTYNNHLYKQISNGTSGGAT
NDNTYFGYSTPWGYFDFNRFHCHFSPRDWQRLINNNWGFRPKRLS
FKLFNIQVKEVTQNEGTKTIANNLTSTIQVFTDSEYQLPYVLGSA
HQGCLPPFPADVFMIPQYGYLTLNNGSQAVGRSSFYCLEYFPSQM
LRTGNNFQFTYTFEDVPFHSSYAHSQSLDRLMNPLIDQYLYYLSR
TQTTGGTANTQTLGFSQGGPNTMANQAKNWLPGPCYRQQRVSTTT
GQNNNSNFAWTAGTKYHLNGRNSLANPGIAMATHKDDEERFFPSN
GILIFGKQNAARDNADYSDVMLTSEEEIKTTNPVATEEYGIVADN
LQQQNLADQDYTKTATAPQIGTVNSQGALPGMVWQNRDVYLQGPI
WAKIPHTDGNFHPSPLMGGFGLKHPPPQILIKNTPVPADPPTTFN
QSKLNSFITQYSTGQVSVEIEWELQKENSKRWNPEIQYTSNYYKS
TSVDFAVNTEGVYSEPRPIGTRYLTRNL
rh10-retro
(SEQ ID NO: 57)
MAADGYLPDWLEDNLSEGIREWWDLKPGAPKPKANQQKQDDGRGL
VLPGYKYLGPFNGLDKGEPVNAADAAALEHDKAYDQQLKAGDNPY
LRYNHADAEFQERLQEDTSFGGNLGRAVFQAKKRVLEPLGLVEEG
AKTAPGKKRPVEPSPQRSPDSSTGIGKKGQQPAKKRLNFGQTGDS
ESVPDPQPIGEPPAGPSGLGSGTMAAGGGAPMADNNEGADGVGSS
SGNWHCDSTWLGDRVITTSTRTWALPTYNNHLYKQISNGTSGGST
NDNTYFGYSTPWGYFDFNRFHCHFSPRDWQRLINNNWGFRPKRLN
FKLFNIQVKEVTQNEGTKTIANNLTSTIQVFTDSEYQLPYVLGSA
HQGCLPPFPADVFMIPQYGYLTLNNGSQAVGRSSFYCLEYFPSQM
LRTGNNFEFSYQFEDVPFHSSYAHSQSLDRLMNPLIDQYLYYLSR
TQSTGGTAGTQQLLFSQAGPNNMSAQAKNWLPGPCYRQQRVSTTL
SQNNNSNFAWTGATKYHLNGRDSLVNPGVAMATHKDDEERFFPSS
GVLMFGKQGAGKDNVDYSSVMLTSEEEIKTTNPVATEQYGVVADN
LQQQNLADQDYTKTAAAPIVGAVNSQGALPGMVWQNRDVYLQGPI
WAKIPHTDGNFHPSPLMGGFGLKHPPPQILIKNTPVPADPPTTFS
QAKLASFITQYSTGQVSVEIEWELQKENSKRWNPEIQYTSNYYKS
TNVDFAVNTDGTYSEPRPIGTRYLTRNL
AAV2
(SEQ ID NO: 64)
MAADGYLPDWLEDTLSEGIRQWWKLKPGPPPPKPAERHKDDSRGL
VLPGYKYLGPFNGLDKGEPVNEADAAALEHDKAYDRQLDSGDNPY
LKYNHADAEFQERLKEDTSFGGNLGRAVFQAKKRVLEPLGLVEEP
VKTAPGKKRPVEHSPVEPDSSSGTGKAGQQPARKRLNFGQTGDAD
SVPDPQPLGQPPAAPSGLGTNTMATGSGAPMADNNEGADGVGNSS
GNWHCDSTWMGDRVITTSTRTWALPTYNNHLYKQISSQSGASNDN
HYFGYSTPWGYFDFNRFHCHFSPRDWQRLINNNWGFRPKRLNFKL
FNIQVKEVTQNDGTTTIANNLTSTVQVFTDSEYQLPYVLGSAHQG
CLPPFPADVFMVPQYGYLTLNNGSQAVGRSSFYCLEYFPSQMLRT
GNNFTFSYTFEDVPFHSSYAHSQSLDRLMNPLIDQYLYYLSRTNT
PSGTTTQSRLQFSQAGASDIRDQSRNWLPGPCYRQQRVSKTSADN
NNSEYSWTGATKYHLNGRDSLVNPGPAMASHKDDEEKFFPQSGVL
IFGKQGSEKTNVDIEKVMITDEEEIRTTNPVATEQYGSVSTNLQR
GNRQAATADVNTQGVLPGMVWQDRDVYLQGPIWAKIPHTDGHFHP
SPLMGGFGLKHPPPQILIKNTPVPANPSTTFSAAKFASFITQYST
GQVSVEIEWELQKENSKRWNPEIQYTSNYNKSVNVDFTVDTNGVY
SEPRPIGTRYLTRNL
AAV2-retro
(SEQ ID NO: 65)
MAADGYLPDWLEDTLSEGIRQWWKLKPGPPPPKPAERHKDDSRGL
VLPGYKYLGPFNGLDKGEPVNEADAAALEHDKAYDRQLDSGDNPY
LKYNHADAEFQERLKEDTSFGGNLGRAVFQAKKRVLEPLGLVEEP
VKTAPGKKRPVEHSPVEPDSSSGTGKAGQQPARKRLNFGQTGDAD
SVPDPQPLGQPPAAPSGLGTNTMATGSGAPMADNNEGADGVGNSS
GNWHCDSTWMGDRVITTSTRTWALPTYNNHLYKQISSQSGASNDN
HYFGYSTPWGYFDFNRFHCHFSPRDWQRLINNNWGFRPKRLNFKL
FNIQVKEVTQNDGTTTIANNLTSTVQVFTDSEYQLPYVLGSAHQG
CLPPFPADVFMVPQYGYLTLNNGSQAVGRSSFYCLEYFPSQMLRT
GNNFTFSYTFEDVPFHSSYAHSQSLDRLMNPLIDQYLYYLSRTNT
PSGTTTQSRLQFSQAGASDIRDQSRNWLPGPCYRQQRVSKTSADN
NNSEYSWTGATKYHLNGRDSLVNPGPAMASHKDDEEKFFPQSGVL
IFGKQGSEKTNVDIEKVMITDEEEIRTTNPVATEQYGSVSTNLQR
GNLADQDYTKTARQAATADVNTQGVLPGMVWQDRDVYLQGPIWAK
IPHTDGHFHPSPLMGGFGLKHPPPQILIKNTPVPANPSTTFSAAK
FASFITQYSTGQVSVEIEWELQKENSKRWNPEIQYTSNYNKSVNV
DFTVDTNGVYSEPRPIGTRYLTRNL

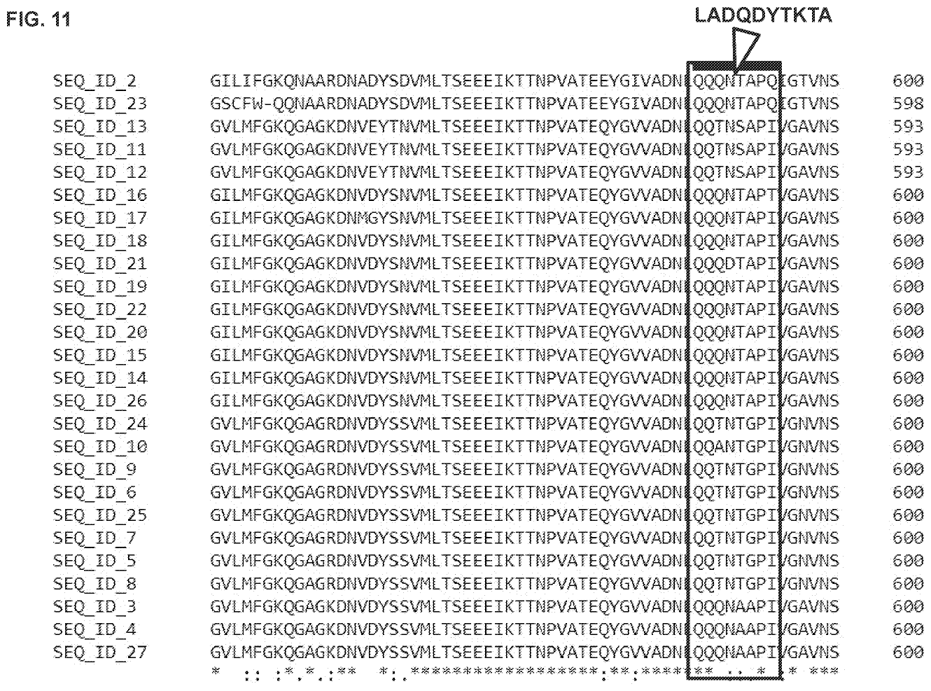

A subject retrograde-enhanced clade E variant AAV capsid protein includes the amino acid sequence LADQDYTKTA (SEQ ID NO: 30) inserted into a clade E AAV capsid protein. In some cases, the sequence is inserted such that it immediately follows a QQQN (SEQ ID NO: 28), QQTN (SEQ ID NO: 29), QQQD (SEQ ID NO: 30), or QQAN (SEQ ID NO: 31) sequence (see FIG. 11 for examples). In some cases, the sequence is inserted such that it immediately follows a QQQN (SEQ ID NO: 28) sequence. In some cases, the sequence is inserted such that it immediately precedes a TAPQ (SEQ ID NO: 58), SAPI (SEQ ID NO: 59), TAPT (SEQ ID NO: 60), TAPI (SEQ ID NO: 61), TGPI (SEQ ID NO: 62), or AAPI (SEQ ID NO: 63) sequence (see FIG. 11 for examples). In some cases, the sequence is inserted such that it immediately precedes a TAPQ (SEQ ID NO: 58) sequence.

In some cases, the LADQDYTKTA (SEQ ID NO: 30) sequence is inserted such that it immediately follows a QQQN (SEQ ID NO: 28), QQTN (SEQ ID NO: 29), QQQD (SEQ ID NO: 30), or QQAN (SEQ ID NO: 31) sequence, and immediately precedes a TAPQ (SEQ ID NO: 58), SAPI (SEQ ID NO: 59), TAPT (SEQ ID NO: 60), TAPI (SEQ ID NO: 61), TGPI (SEQ ID NO: 62), or AAPI (SEQ ID NO: 63) sequence. In some cases, the sequence is inserted such that it immediately follows a QQQN (SEQ ID NO: 28) sequence and immediately precedes a TAPQ (SEQ ID NO: 58) sequence. In some cases, the sequence is inserted such that it immediately follows a QQQN (SEQ ID NO: 28) sequence and immediately precedes a AAPI (SEQ ID NO: 63) sequence.

By adeno-associated virus, or “AAV” it is meant the virus itself or derivatives thereof. The term covers all subtypes and both naturally occurring and recombinant forms, except where required otherwise. Examples include, but are not limited to: AAV type 1 (AAV1), AAV type 2 (AAV2), AAV type 3 (AAV3), AAV type 4 (AAV4), AAV type 5 (AAV5), AAV type 6 (AAV6), AAV type 7 (AAV7), AAV type 8 (AAV8), AAV type 9 (AAV9), AAV 9_hu14, AAVrh10, avian AAV, bovine AAV, canine AAV, equine AAV, primate AAV, non-primate AAV, and ovine AAV. “Primate AAV” refers to AAV capable of infecting primates, “non-primate AAV” refers to AAV capable of infecting non-primate mammals, “bovine AAV” refers to AAV capable of infecting bovine mammals, etc.

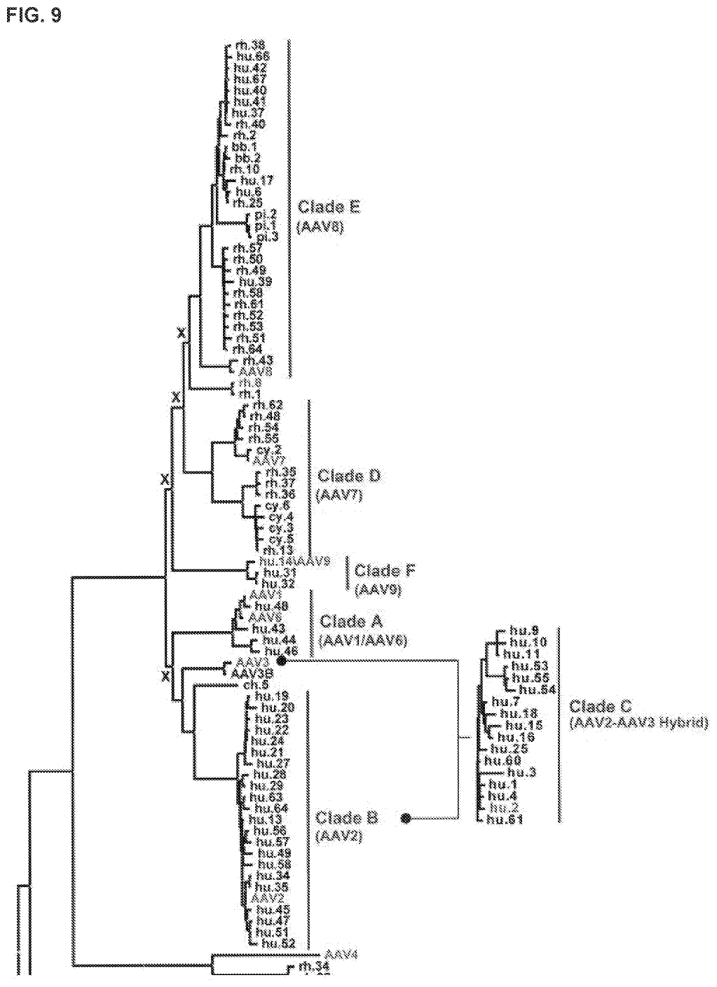

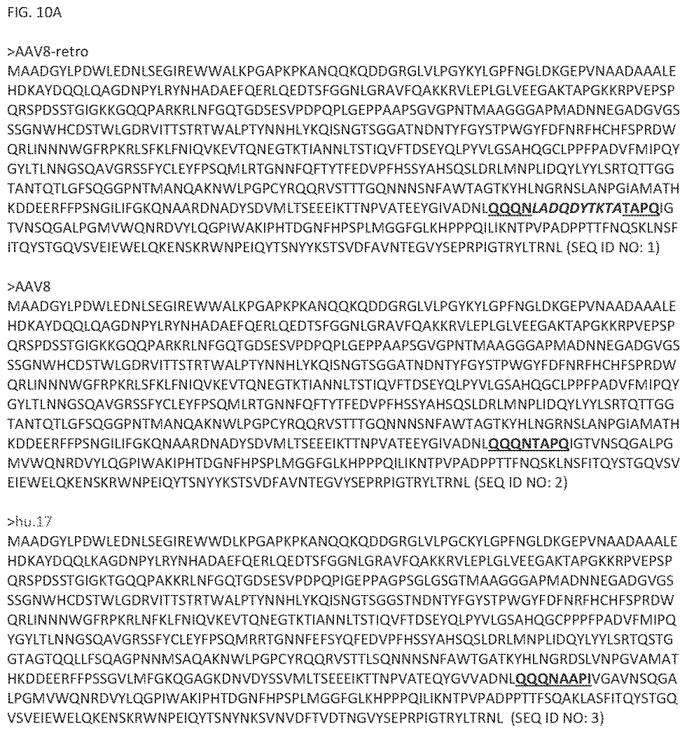

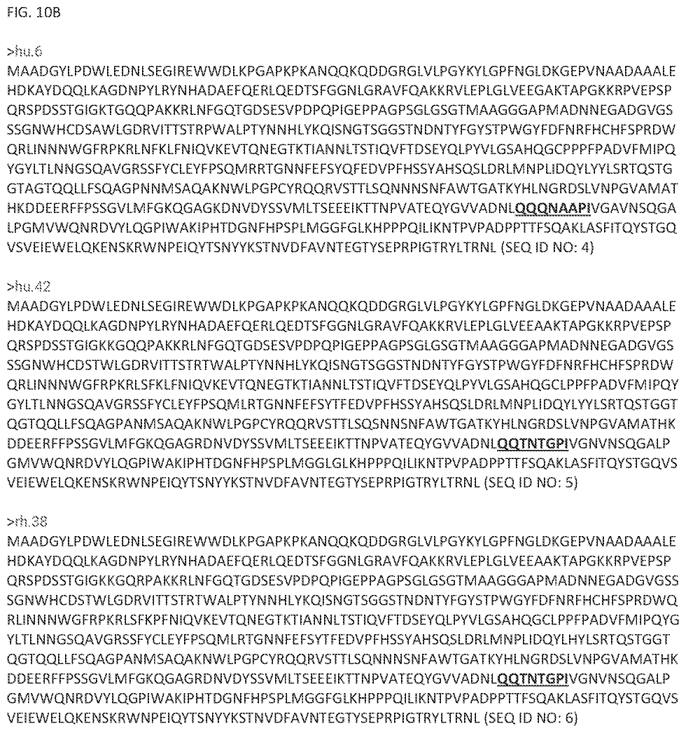

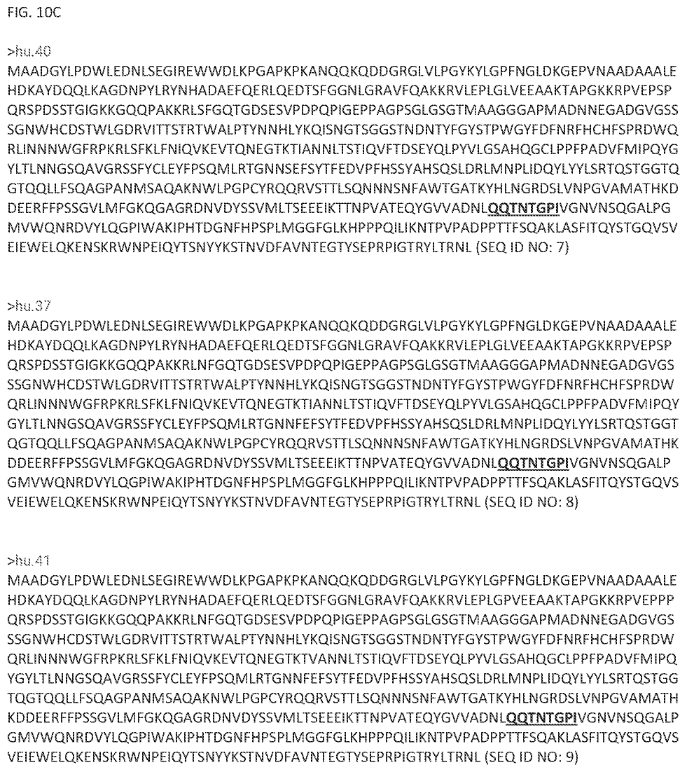

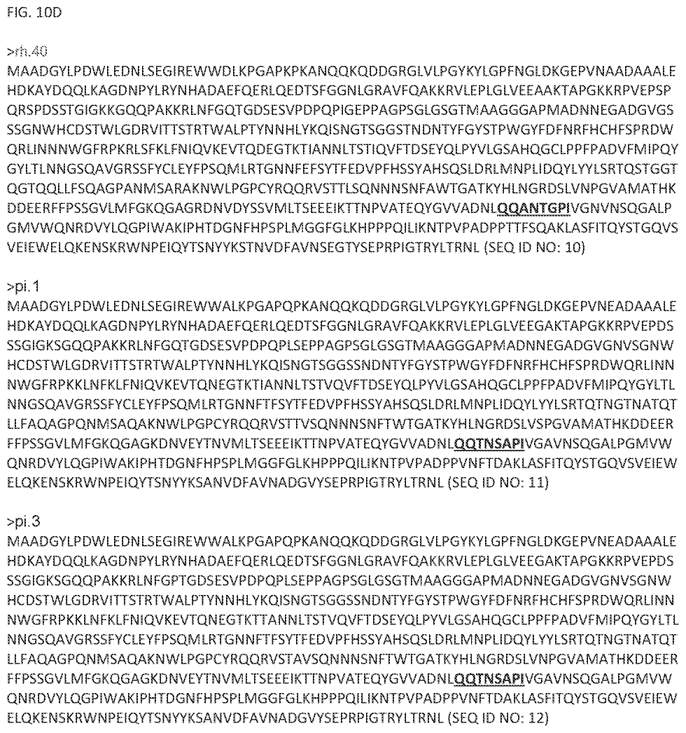

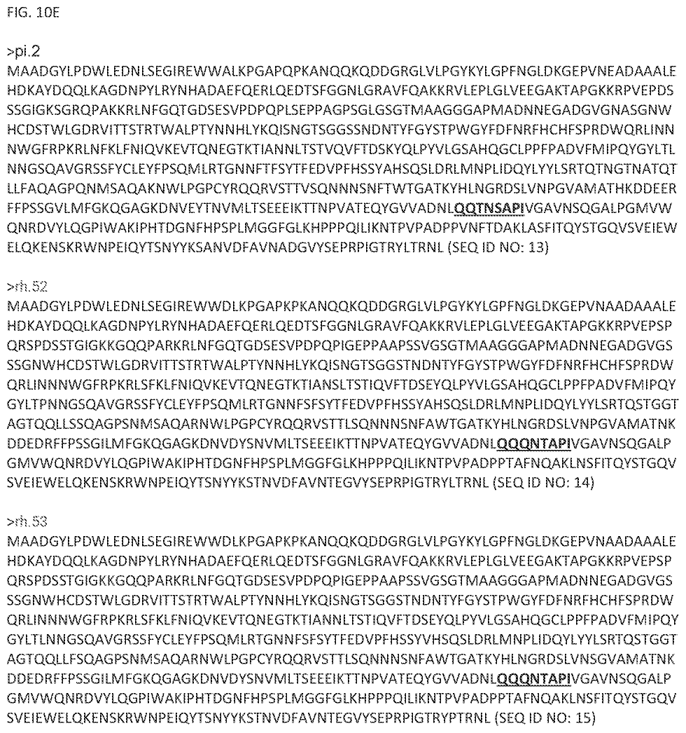

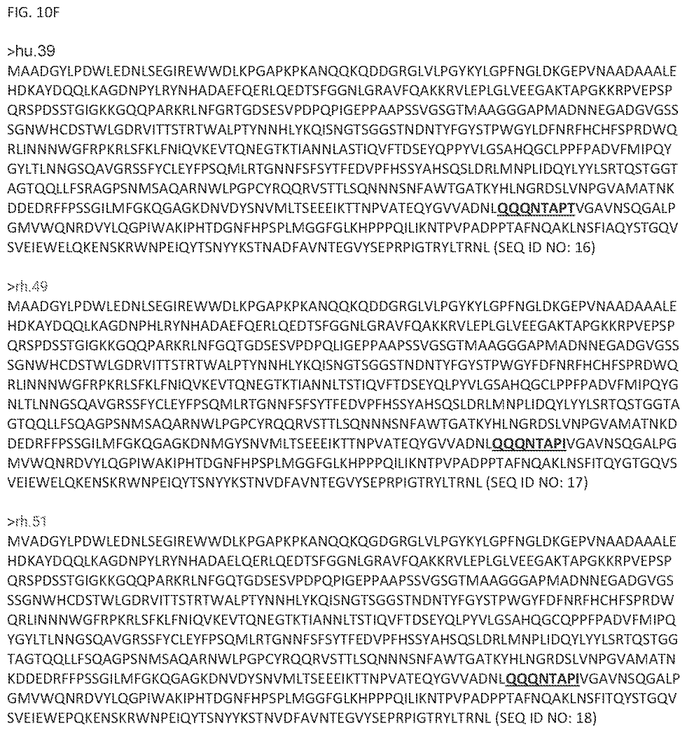

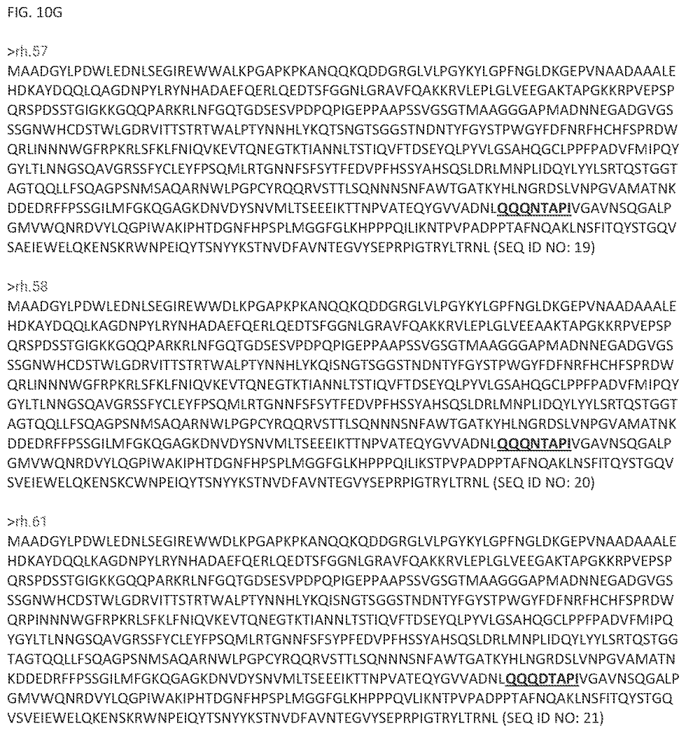

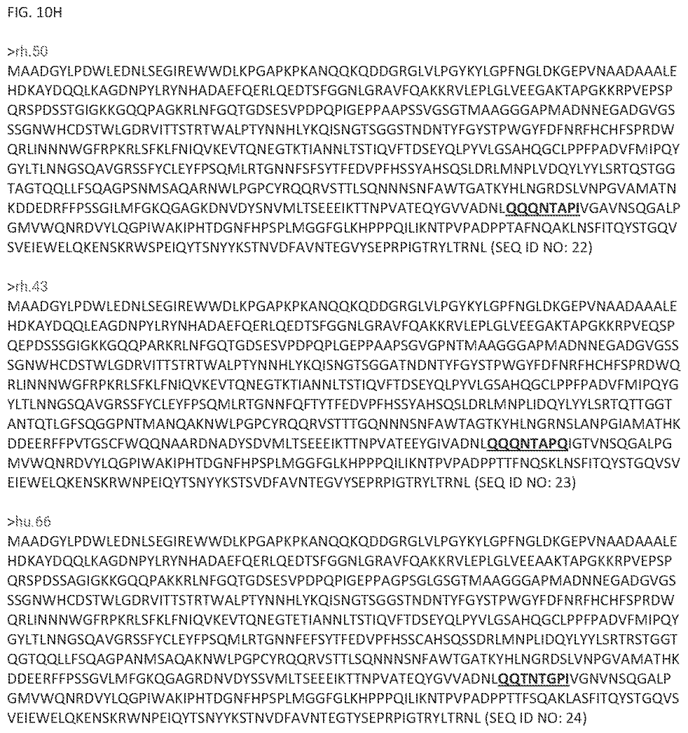

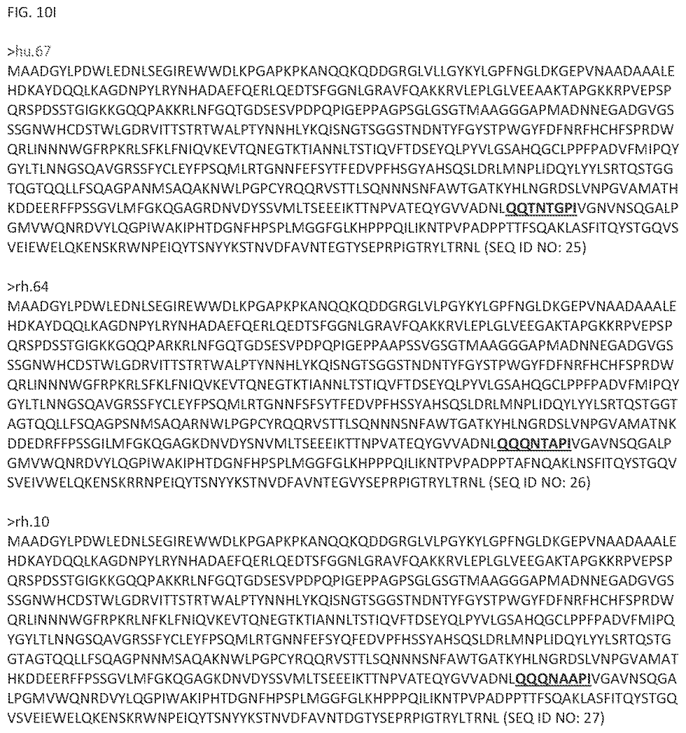

AAVs can be classified into clades (see, e.g., FIG. 9; Gao et. al., J Virol. 2004 June; 78(12): 6381-6388; U.S. Pat. No. 7,906,111, and U.S. Published Patent Application No. US 2003/0138772, the disclosures of which are incorporated herein by reference with respect to AAV sequences and their classification into various clades, especially the sequences and names of AAVs of Clade E).

In some embodiments, a subject AAV is a retrograde-enhanced variant of a clade E AAV. As such, in some cases a subject AAV capsid protein is a retrograde-enhanced variant of a clade E AAV capsid protein. Examples of clade E AAVs, their associated capsid proteins, and retro-grade enhanced variants thereof include, but are not limited to those listed in Table 1.

TABLE 1
Examples of Clade E and Clade E-retro capsid sequences

| | Capsid protein | | Capsid protein |
|---|---|---|---|
| | SEQ ID NO: | | SEQ ID NO: |
| AAV | (Before insertion) | AAV-retro | (After insertion) |
| AAV8 | 2 | AAV8-retro | 32 |
| hu17 | 3 | hu17-retro | 33 |
| hu6 | 4 | hu6-retro | 34 |
| hu42 | 5 | hu42-retro | 35 |
| rh38 | 6 | rh38-retro | 36 |
| hu40 | 7 | hu40-retro | 37 |
| hu37 | 8 | hu37-retro | 38 |
| hu41 | 9 | hu41-retro | 39 |
| rh40 | 10 | rh40-retro | 40 |
| pi1 | 11 | pi1-retro | 41 |
| pi3 | 12 | pi3-retro | 42 |
| pi2 | 13 | pi2-retro | 43 |
| rh52 | 14 | rh52-retro | 44 |
| rh53 | 15 | rh53-retro | 45 |
| hu39 | 16 | hu39-retro | 46 |
| rh49 | 17 | rh49-retro | 47 |
| rh51 | 18 | rh51-retro | 48 |
| rh57 | 19 | rh57-retro | 49 |
| rh58 | 20 | rh58-retro | 50 |
| rh61 | 21 | rh61-retro | 51 |
| rh50 | 22 | rh50-retro | 52 |
| rh43 | 23 | rh43-retro | 53 |
| hu66 | 24 | hu66-retro | 54 |
| hu67 | 25 | hu67-retro | 55 |
| rh64 | 26 | rh64-retro | 56 |
| rh10 | 27 | rh10-retro | 57 |

The term “substantially identical” in the context of variant AAV capsid polypeptides and non-variant parent capsid polypeptides refers to sequences with 1 or more amino acid changes. In some embodiments, these changes do not affect the packaging function of the capsid polypeptides. In some embodiments, substantially identical include variant AAV capsid polypeptides about 99%, about 98%, about 97%, about 96%, about 95%, about 94%, about 93%, about 92%, about 91%, or about 90% identical to non-variant parent capsid polypeptides. In some embodiments, the variant AAV capsid polypeptides can be substantially identical to non-variant parent capsid polypeptides over a subregion of the variant AAV capsid polypeptide, such as over about 25%, about 50%, about 75%, or about 90% of the total polypeptide sequence length.

In some cases, the variant clade E AAV capsid protein into which LADQDYTKTA (SEQ ID NO: 30) is inserted includes an amino acid sequence that has 80% or more identity (e.g., 85% or more, 90% or more, 95% or more, 97% or more, 98% or more, 99% or more, 99.5% or more, or 100% identity) with the clade E AAV capsid protein amino acid sequence set forth in any one of SEQ ID Nos: 2-27 (see Table 1). In some cases, the variant clade E AAV capsid protein into which LADQDYTKTA (SEQ ID NO: 30) is inserted includes an amino acid sequence that has 90% or more identity (e.g., 95% or more, 97% or more, 98% or more, 99% or more, 99.5% or more, or 100% identity) with the clade E AAV capsid protein amino acid sequence set forth in any one of SEQ ID Nos: 2-27. In some cases, the variant clade E AAV capsid protein into which LADQDYTKTA (SEQ ID NO: 30) is inserted includes the amino acid sequenced set forth as any one of SEQ ID Nos: 2-27. In some cases, the variant clade E AAV capsid protein into which LADQDYTKTA (SEQ ID NO: 30) is inserted is an AAV8 capsid protein (e.g., SEQ ID NO: 2). In some cases, the variant clade E AAV capsid protein into which LADQDYTKTA (SEQ ID NO: 30) is inserted is an rh10 capsid protein (e.g., SEQ ID NO: 27). In some cases, the variant clade E AAV capsid protein into which LADQDYTKTA (SEQ ID NO: 30) is inserted is an AAV8 capsid protein (e.g., SEQ ID NO: 2) or an rh10 capsid protein (e.g., SEQ ID NO: 27).

The right side of Table 1 (SEQ ID Nos: 32-57) lists the resulting retrograde-enhanced clade E variant AAV capsid proteins when the LADQDYTKTA (SEQ ID NO: 30) sequence is inserted into the sequences listed on the left side of the table (SEQ ID Nos 2-27) as depicted in FIG. 11. In some cases, a subject retrograde-enhanced clade E variant AAV capsid protein includes an amino acid sequence that has 80% or more identity (e.g., 85% or more, 90% or more, 95% or more, 97% or more, 98% or more, 99% or more, 99.5% or more, or 100% identity) with the amino acid sequence set forth in any one of SEQ ID Nos: 32-57 (see Table 1). In some cases, a subject retrograde-enhanced clade E variant AAV capsid protein includes an amino acid sequence that has 90% or more identity (e.g., 95% or more, 97% or more, 98% or more, 99% or more, 99.5% or more, or 100% identity) with the clade E AAV capsid protein amino acid sequence set forth in any one of SEQ ID Nos: 32-57. In some cases, a subject retrograde-enhanced clade E variant AAV capsid protein includes the amino acid sequence set forth as SEQ ID NO: 32. In some cases, a subject retrograde-enhanced clade E variant AAV capsid protein includes the amino acid sequence set forth as SEQ ID NO: 57. In some cases, a subject retrograde-enhanced clade E variant AAV capsid protein includes the amino acid sequence set forth as SEQ ID NO: 32 or SEQ ID NO: 57.

The abbreviation “rAAV” refers to recombinant adeno-associated virus, also referred to as a recombinant AAV vector (or “rAAV vector”). The term “variant” is used herein with respect to the term AAV (e.g., an AAV viral particle, AAV vector, AAV capsid) refers to a AAV virion, vector, or capsid, in which the capsid protein is a non-naturally occurring capsid. A variant “AAV vector” as use herein refers to a nucleic acid sequence encoding a variant capsid polypeptide (i.e., the AAV vector comprises a nucleic acid sequence encoding a variant capsid polypeptide, also referred to as a variant AAV capsid protein or variant AAV capsid polypeptide—the terms “polypeptide” and “protein” are used interchangeably herein). The subject variant AAV capsid polypeptides discussed herein exhibit (provide for) enhanced retrograde transduction.

By a “recombinant AAV vector”, or “rAAV vector” it is meant an AAV virus or AAV viral chromosomal material comprising a polynucleotide sequence not of AAV origin (i.e., a polynucleotide heterologous to AAV), typically a nucleic acid sequence of interest (a transgene sequence) to be introduced into a target cell. In general, the heterologous polynucleotide is flanked by AAV inverted terminal repeat sequences (ITRs). In some instances, the recombinant viral vector also comprises viral genes important for the packaging of the recombinant viral vector material. By “packaging” it is meant a series of intracellular events resulting in the assembly of AAV virions (AAV viral particles) which encapsidate a nucleic acid sequence (e.g., a transgene sequence). Packaging can refer to encapsidation of a transgene sequence into a capsid such as a variant AAV capsid polypeptide described herein. Examples of nucleic acid sequences important for AAV packaging (i.e., “packaging genes”) include the AAV “rep” and “cap” genes, which encode for replication and encapsidation proteins of adeno-associated virus, respectively. The term rAAV vector encompasses both rAAV vector particles and rAAV vector plasmids.

A “viral particle” (e.g., AAV viral particle) or “virion” (e.g., AAV virion) or “virus” (e.g., AAV virus) refers to an individual unit of virus that includes a capsid encapsidating a virus-based polynucleotide, e.g. the viral genome (as in a wild type virus), or, e.g., a nucleic with a transgene sequence (as in a recombinant virus). An “AAV viral particle” refers to a viral particle composed of at least one AAV capsid protein (e.g., a subject retrograde-enhanced clade E variant AAV capsid protein) and an encapsidated polynucleotide AAV vector (e.g., rAAV vector). If the particle comprises a heterologous polynucleotide (i.e., a polynucleotide other than a wild-type AAV genome, such as a transgene to be delivered to a mammalian cell), it is typically referred to as an “rAAV vector particle” or simply an “rAAV vector”. Thus, production of rAAV particle necessarily includes production of rAAV vector, as such a vector is contained within an rAAV particle.

### Transgene Sequence

As noted above, AAV vectors can include a heterologous nucleic acid sequence not of AAV origin (e.g., as part of the nucleic acid insert). The heterologous nucleic acid sequence typically includes a sequence of interest (a transgene sequence).

Thus, in some cases a subject rAAV particle (e.g., a subject retrograde enhances AAV), in addition to including a variant AAV capsid protein, also includes (e.g., encapsidates) a nucleic acid that includes a transgene sequence. The transgene sequence can be operably linked to a control element (such as a promoter—in which case the combination can be referred to as an expression cassette) in a manner permitting transcription, translation and/or expression in a cell transfected with the AAV vector or infected with the AAV virion produced according to the present disclosure.

Expression control sequences include appropriate transcription initiation, termination, promoter and enhancer sequences; efficient RNA processing signals such as splicing and polyadenylation (polyA) signals; sequences that stabilize cytoplasmic mRNA; sequences that enhance translation efficiency (i.e., Kozak consensus sequence); sequences that enhance protein stability; and when desired, sequences that enhance secretion of the encoded product. A great number of expression control sequences, including promoters selected from native, constitutive, inducible and/or tissue-specific, are known in the art and may be utilized.

Examples of constitutive promoters include, without limitation, the retroviral Rous sarcoma virus (RSV) LTR promoter (optionally with the RSV enhancer), the cytomegalovirus (CMV) promoter (optionally with the CMV enhancer) (see, e.g., Boshart et al., Cell, 41:521-530 (1985)), the SV40 promoter, the dihydrofolate reductase promoter, the beta-actin promoter, the phosphoglycerol kinase (PGK) promoter, and the EF1 promoter (Invitrogen). Inducible promoters allow regulation of gene expression and can be regulated by exogenously supplied compounds, environmental factors such as temperature, or the presence of a specific physiological state, e.g., acute phase, a particular differentiation state of the cell, or in replicating cells only. Inducible promoters and inducible systems are available from a variety of commercial sources, including, without limitation, Invitrogen, Clonetech and Ariad. Many other systems have been described and can be readily selected by one of skill in the art. Examples of inducible promoters regulated by exogenously supplied compounds, include, the zinc-inducible sheep metallothionine (MT) promoter, the dexamethasone (Dex)-inducible mouse mammary tumor virus (MMTV) promoter, the 77 polymerase promoter system (WO 98/10088); the ecdysone insect promoter (No et al., (1996) Proc. Natl. Acad. Sci. USA, 93:3346-3351), the tetracycline-repressible system (Gossen et al., (1992) Proc. Natl. Acad. Sci. USA, 89:5547-5551), the tetracycline-inducible system (Gossen et al., (1995) Science, 268:1766-1769, see also Harvey et al., (1998) Curr. Opin. Chem. Biol., 2:512-518), the RU486-inducible system (Wang et al., (1997) Nat. Biotech., 15:239-243 and Wang et al., (1997) Gene Ther., 4:432-441) and the rapamycin-inducible system (Magari et al., (1997) J. Clin. Invest., 100:2865-2872). Other types of inducible promoters useful in this context are those regulated by a specific physiological state, e.g., temperature, acute phase, a particular differentiation state of the cell, or in replicating cells only.

In some cases a nucleotide sequence of interest is operably linked to a tissue-specific promoter. For instance, if expression in skeletal muscle is desired, a promoter active in muscle should be used. These include the promoters from genes encoding skeletal .beta.-actin, myosin light chain 2A, dystrophin, muscle creatine kinase, as well as synthetic muscle promoters with activities higher than naturally-occurring promoters (see Li et al., Nat. Biotech., 17:241-245 (1999)). Examples of promoters that are tissue-specific are known for liver (albumin, Miyatake et al., (1997) J. Virol., 71:5124-32; hepatitis B virus core promoter, Sandig et al., (1996) Gene Ther., 3:1002-9; alpha-fetoprotein (AFP), Arbuthnot et al., (1996) Hum. Gene Ther., 7:1503-14), bone osteocalcin (Stein et al., (1997) Mol. Biol. Rep., 24:185-96); bone sialoprotein (Chen et al., (1996) J. Bone Miner. Res., 11:654-64), lymphocytes (CD2, Hansal et al., (1998) J. Immunol., 161:1063-8; immunoglobulin heavy chain; T cell receptor chain), neuronal such as neuron-specific enolase (NSE) promoter (Andersen et al., (1993) Cell. Mol. Neurobiol., 13:503-15), neurofilament light-chain gene (Piccioli et al., (1991) Proc. Natl. Acad. Sci. USA, 88:5611-5), and the neuron-specific vgf gene (Piccioli et al., (1995) Neuron, 15:373-84), among others.

Useful heterologous control sequences generally include those derived from sequences encoding mammalian or viral genes. Examples include, but are not limited to, the SV40 early promoter, mouse mammary tumor virus long terminal repeat (LTR) promoter; adenovirus major late promoter (Ad MLP); a herpes simplex virus (HSV) promoter, an endogenous cellular promoter heterologous to the gene of interest, a cytomegalovirus (CMV) promoter such as the CMV immediate early promoter region (CMVIE), a rous sarcoma virus (RSV) promoter, synthetic promoters, hybrid promoters, and the like. In addition, sequences derived from nonviral genes, such as the murine metallothionein gene, can also be used. Such promoter sequences are commercially available from, e.g., Stratagene (San Diego, Calif.). In some embodiments, a cell type-specific or a tissue-specific promoter can be operably linked to the nucleotide sequence of interest and allowing for selective or preferential expression in a particular cell type(s) or tissue(s). Thus, in some embodiments, an inducible promoter can be operably linked to the transgene sequence.

In some cases a nucleic acid that includes a transgene sequence is packaged with the variant AAV capsid polypeptides of the disclosure. In some embodiments, the nucleic acid is at least 50, 100, 200, 300, 400, 500, 600, 700, 800, 900, 1000, 1100, 1200, 1300, 1400, or 1500 nucleotides (nt) in length. In some embodiments, the nucleic acid is 50 nucleotides to 4000 nucleotides long (e.g., 50-3000, 50-2000, 50-1500, 50-1200, 50-1000, 50-900, 50-750, 50-500, 100-4000, 100-3000, 100-2000, 100-1500, 100-1200, 100-1000, 100-900, 100-750, 100-500, 300-4000, 300-3000, 300-2000, 300-1500, 300-1200, 300-1000, 300-900, 300-750, 300-500, 500-4000, 500-3000, 500-2000, 500-1500, 500-1200, 500-1000, or 500-900 nt long). In some embodiments, the transgene sequence is at least 50, 100, 200, 300, 400, 500, 600, 700, 800, 900, 1000, 1100, 1200, 1300, 1400, or 1500 nucleotides (nt) in length. In some embodiments, the transgene sequence is 50 nucleotides to 4000 nucleotides long (e.g., 50-3000, 50-2000, 50-1500, 50-1200, 50-1000, 50-900, 50-750, 50-500, 100-4000, 100-3000, 100-2000, 100-1500, 100-1200, 100-1000, 100-900, 100-750, 100-500, 300-4000, 300-3000, 300-2000, 300-1500, 300-1200, 300-1000, 300-900, 300-750, 300-500, 500-4000, 500-3000, 500-2000, 500-1500, 500-1200,500-1000, or 500-900 nt long).

In some embodiments, an AAV vector packaged by a variant AAV capsid polypeptide is at least about 2000 nucleotides in total length and up to about 5000 nucleotides in total length. In some embodiments, an AAV vector packaged by the variant AAV capsid polypeptides is about 2000 nucleotides, about 2400 nucleotides, about 2800 nucleotides, about 3000 nucleotides, about 3200 nucleotides, about 3400 nucleotides, about 3600 nucleotides, about 3800 nucleotides, about 4000 nucleotides, about 4200 nucleotides, about 4400 nucleotides, about 4600 nucleotides, about 4700 nucleotides, or about 4800 nucleotides. In some embodiments, an AAV vector packaged by the variant AAV capsid polypeptides is between about 2000 nucleotides (2 kb) and about 5000 nucleotides (5 kb). In some embodiments, an AAV vector packaged by the variant AAV capsid polypeptides is between about 2400 nucleotides (2.4 kb) and about 4800 nucleotides (4.8 kb). In some embodiments, an AAV vector packaged by the variant AAV capsid polypeptides is between about 3000 nucleotides (3 kb) and about 5000 nucleotides (5 kb). In some embodiments, an AAV vector packaged by the variant AAV capsid polypeptides is between about 3000 nucleotides (3 kb) and about 4000 nucleotides (4 kb).

The nucleotide sequence of interest (transgene sequence) can be any desired sequence. Examples include, but are not limited to: a sequence that encodes a non-coding RNA (e.g., a CRISPR/Cas guide RNA, an RNAi agent such as an shRNA, an antisense RNA, a ribozyme, and the like), a sequence that encodes a protein (an mRNA), an expression cassette (which includes a promoter sequence that is operably linked to a protein-coding sequence or a non-coding RNA sequence), and a sequence for homology directed repair (e.g., a donor sequence). In some embodiments, the transgene is an expression cassette for a CRISPR/CAS expression system (e.g., including a CRISPR/Cas guide RNA and a CRISPR/Cas effector protein such as Cas9 or Cas12). In some cases, the transgene encodes a CRISPR/Cas effector protein such as a type II (e.g., Cas9) or type V (Cas12) effector protein. In some embodiments, the transgene sequence encodes an RNAi agent (e.g., shRNA, siRNA, miRNA).

As noted above, in some cases a subject transgene sequence encodes an RNAi agent. Such an agent can target a particular cellular target sequence. For example, in some cases the RNAi agent (e.g., an shRNA) targets CamKv. Any convenient version of RNAi agent can be used. For example, endogenous miRNA sequences can be used as a scaffold for artificial miRNA. For example, in some cases an shRNA of interested can be embedded in a miRNA backbone such as a mir-155 backbone (see, e.g., Fowler et al., Nucleic Acids Res. 2016 Mar. 18; 44(5): e48, “Improved knockdown from artificial microRNAs in an enhanced miR-155 backbone: a designer's guide to potent multi-target RNAi”; and Uva et al., RNA. 2013 March; 19(3):365-79, “Rat mir-155 generated from the lncRNA Bic is ‘hidden’ in the alternate genomic assembly and reveals the existence of novel mammalian miRNAs and clusters”—both of which references are hereby incorporated by reference in their entirety). See the working examples below for such an example that was used for targeting CamKv. As such, in some cases, a subject transgene sequence encodes an RNAi agent that targets CamKv. In some cases, a subject transgene sequence encoded an shRNA that targets CamKv and is embedded in a microRNA backbone such as a miR-155 backbone. In some cases, the transgene includes the sequence depicted in FIG. 12 (which includes a CamKv targeting sequence embedded in a miR-155 backbone). In some cases, the shRNA-encoding sequence includes a sequence that has 90% or more (e.g., 95% or more, 97% or more, 98% or more, 99% or more, or 99.5% or more) sequence identity with the sequence depicted in FIG. 12 (which includes a CamKv targeting sequence embedded in a miR-155 backbone). In some cases, the transgene includes the CamKv-shRNA encoding portion of the sequence depicted in FIG. 12, which includes a mismatch in the stem-loop (which, as known in the art, can in some cases increase knockdown efficiency for RNAi agents such as shRNAs). In some cases, the shRNA-encoding sequence includes a sequence that has 90% or more (e.g., 95% or more, 97% or more, 98% or more, 99% or more, or 99.5% or more) sequence identity with the CamKv-shRNA portion of the sequence depicted in FIG. 12. In some cases, the shRNA-encoding sequence targets the same target sequence that the shRNA of FIG. 12 targets. In some cases, a subject shRNA will include one or more mismatches in the stem loop, and in some cases the stem loop will not have a mismatch (i.e., will be a perfect duplex).

In some cases a subject nucleotide sequence of interest encodes a non-coding RNA (e.g., a CRISPR/Cas guide RNA, an antisense RNA, a ribozyme, an shRNA, a microRNA, an aptamer).

In some cases a subject transgene sequence encodes a protein (e.g., a therapeutic protein meant to alleviate a disease and/or its symptoms, a genome-editing enzyme such as a CRISPR/Cas effector protein, TALEN, Zinc Finger nuclease, etc. - meant to provide for targeted genome editing, etc.). Examples of proteins that can be encoded by a transgene include but are not limited to selectable markers and reporter genes, e.g., sequences encoding geneticin, hygromycin or puromycin resistance, among others. Selectable markers and reporter genes can be used to signal the presence of the plasmids/vectors in bacterial cells, including, for example, examining ampicillin resistance.

In some cases, a transgene encodes a genome-targeting protein. Non-limiting examples of targeted proteins (genome-targeting proteins) that can be encoded by a transgene sequence include naturally occurring and recombinant nucleases, e.g. restriction endonucleases, meganucleases, homing endonucleases, and CRISPR/Cas effector proteins (e.g., CRISPR/Cas endonucleases such as Cas9, Cas12, Cas13, and the like—or variants thereof such as nickase variants, nuclease inactivated variants, fusion variants in which an effector protein is fused to a functional domain such as a transcriptional activator or repressor or chromatin modifier, and the like), Zinc finger nucleases (ZFNs) and transcriptional activation or suppression fusion variants thereof, and Transcription Activator-Like Effector Nucleases (TALENs) and transcriptional activation or suppression fusion variants thereof. In some cases a nucleic includes a sequence that encodes a CRISPR/Cas effector protein and a CRISPR/Cas guide RNA.

In some embodiments, exemplary polypeptides that can be encoded by a subject transgene sequence include neuroprotective polypeptides and/or anti-angiogenic polypeptides (both of which are therapeutic polypeptides). Suitable polypeptides include, but are not limited to, glial derived neurotrophic factor (GDNF), fibroblast growth factor 2 (FGF-2), neurturin, ciliary neurotrophic factor (CNTF), nerve growth factor (NGF; e.g., nerve growth factor-.beta.), brain derived neurotrophic factor (BDNF), neurotrophin-3 (NT-3), neurotrophin-4 (NT-4), neurotrophin-6 (NT-6), epidermal growth factor (EGF), pigment epithelium derived factor (PEDF), a Wnt polypeptide, soluble Fit-1, angiostatin, endostatin, VEGF, an anti-VEGF antibody, a soluble VEGFR, Factor VIII (FVIII), Factor IX (FIX), and a member of the hedgehog family (sonic hedgehog, Indian hedgehog, and desert hedgehog, etc.).

In some embodiments, useful therapeutic products encoded by the heterologous nucleic acid sequence (transgene sequence) include hormones and growth and differentiation factors including, without limitation, insulin, glucagon, growth hormone (GH), parathyroid hormone (PTH), growth hormone releasing factor (GRF), follicle stimulating hormone (FSH), luteinizing hormone (LH), human chorionic gonadotropin (hCG), vascular endothelial growth factor (VEGF), angiopoietins, angiostatin, granulocyte colony stimulating factor (GCSF), erythropoietin (EPO), connective tissue growth factor (CTGF), basic fibroblast growth factor (bFGF), acidic fibroblast growth factor (aFGF), epidermal growth factor (EGF), platelet-derived growth factor (PDGF), insulin growth factors I and II (IGF-I and IGF-II), any one of the transforming growth factor alpha superfamily, including TGF.alpha., activins, inhibins, or any of the bone morphogenic proteins (BMP) BMPs 1-15, any one of the heregulin/neuregulin/ARIA/neu differentiation factor (NDF) family of growth factors, nerve growth factor (NGF), brain-derived neurotrophic factor (BDNF), neurotrophins NT-3 and NT-4/5, ciliary neurotrophic factor (CNTF), glial cell line derived neurotrophic factor (GDNF), neurturin, agrin, any one of the family of semaphorins/collapsins, netrin-1 and netrin-2, hepatocyte growth factor (HGF), ephrins, noggin, sonic hedgehog and tyrosine hydroxylase.

Useful proteins that can be encoded by a transgene sequence include proteins that regulate the immune system including, without limitation, cytokines and lymphokines such as thrombopoietin (TPO), interleukins (IL) IL-1 through IL-25 (including IL-2, IL-4, IL-12 and IL-18), monocyte chemoattractant protein, leukemia inhibitory factor, granulocyte-macrophage colony stimulating factor, Fas ligand, tumor necrosis factors alpha and beta., interferons (alpha, beta, and gamma), stem cell factor, flk-2/flt3 ligand. Gene products produced by the immune system are also useful in the present disclosure. These include, without limitations, immunoglobulins IgG, IgM, IgA, IgD and IgE, chimeric immunoglobulins, humanized antibodies, single chain antibodies, T cell receptors, chimeric T cell receptors, single chain T cell receptors, class I and class II MHC molecules, as well as engineered immunoglobulins and MHC molecules. Useful gene products also include complement regulatory proteins such as complement regulatory proteins, membrane cofactor protein (MCP), decay accelerating factor (DAF), CR1, CF2 and CD59.

Useful proteins that can be encoded by a transgene sequence include any one of the receptors for the hormones, growth factors, cytokines, lymphokines, regulatory proteins and immune system proteins. Useful heterologous nucleic acid sequences also include receptors for cholesterol regulation and/or lipid modulation, including the low density lipoprotein (LDL) receptor, high density lipoprotein (HDL) receptor, the very low density lipoprotein (VLDL) receptor, and scavenger receptors. The disclosure also encompasses the use of gene products such as members of the steroid hormone receptor superfamily including glucocorticoid receptors and estrogen receptors, Vitamin D receptors and other nuclear receptors. In addition, useful gene products include transcription factors such as jun, fos, max, mad, serum response factor (SRF), AP-1, AP-2, myb, MyoD and myogenin, ETS-box containing proteins, TFE3, E2F, ATF1, ATF2, ATF3, ATF4, ZF5, NFAT, CREB, HNF-4 C/EBP, SP1, CCAAT-box binding proteins, interferon regulation factor (IRF-1), Wilms tumor protein, ETS-binding protein, STAT, GATA-box binding proteins, e.g., GATA-3, and the forkhead family of winged helix proteins.

Useful proteins that can be encoded by a transgene sequence include carbamoyl synthetase I, ornithine transcarbamylase, arginosuccinate synthetase, arginosuccinate lyase, arginase, fumarylacetoacetate hydrolase, phenylalanine hydroxylase, alpha-1 antitrypsin, glucose-6-phosphatase, porphobilinogen deaminase, cystathionine beta-synthase, branched chain ketoacid decarboxylase, albumin, isovaleryl-coA dehydrogenase, propionyl CoA carboxylase, methyl malonyl CoA mutase, glutaryl CoA dehydrogenase, insulin, beta-glucosidase, pyruvate carboxylate, hepatic phosphorylase, phosphorylase kinase, glycine decarboxylase, H-protein, T-protein, a cystic fibrosis transmembrane regulator (CFTR) sequence, and a dystrophin cDNA sequence. Still other useful gene products include enzymes useful in enzyme replacement therapy, and which are useful in a variety of conditions resulting from deficient activity of enzyme. For example, enzymes containing mannose-6-phosphate may be utilized in therapies for lysosomal storage diseases (e.g., a suitable gene includes that encoding .beta.-glucuronidase (GUSB)).

In some embodiments, useful gene products encoded by a transgene include non-naturally occurring polypeptides, such as chimeric or hybrid polypeptides having a non-naturally occurring amino acid sequence containing insertions, deletions or amino acid substitutions. For example, single-chain engineered immunoglobulins could be useful in certain immunocompromised patients. Other types of non-naturally occurring gene sequences include antisense molecules and catalytic nucleic acids, such as ribozymes, used to reduce expression of a target.

In some cases, the “nucleotide sequence of interest” (transgene sequence”) (and in some cases an expression cassette that includes the transgene sequence operably linked to a promoter) is flanked by AAV inverted terminal repeat (ITR) sequences, i.e., 5′ and 3′ ITRs, or a variant thereof.

Generally, ITR sequences are about 145 bp in length. The entire sequences encoding the ITRs can be used, although some degree of minor modification of these sequences is permissible. The ability to modify these ITR sequences is within the skill of the art. (See, e.g., texts such as Sambrook et al., “Molecular Cloning. A Laboratory Manual”, 2d ed., Cold Spring Harbor Laboratory, New York (1989); and K. Fisher et al., J Virol., 70:520 532 (1996)). An example of a nucleic acid employed in the present invention is a “cis-acting” plasmid containing the transgene, in which the selected transgene sequence (and in some cases associated regulatory elements) is flanked by the 5′ and 3′ AAV ITR sequences. AAV ITR sequences may be obtained from any known AAV. Examples include, but are not necessarily limited to: AAV1, AAV2, AAV5, AAV6, AAV6.2, AAV7, AAV8, AAV9, AAV10, AAV11, and rh10 ITRs, and variants thereof. In some cases the ITRs are AAV2 ITRs. In some cases the ITRs are ITRs of a clade E AAV. In some cases the ITRs are AAV8 ITRs. In some cases the ITRs are Rh10 ITRs. In some cases, the ITR is a variant ITR. In some cases one ITR is from one source (e.g., AAV2) and the other ITR is from a different source (e.g., AAV8).

In some such cases, the variant ITR lacks a functional terminal resolution site (TRS). The term “lacking a terminal resolution site” can refer to an AAV ITR that includes a mutation (e.g., a substitution mutation, deletion, insertion) that abrogates the function of the terminal resolution site (TRS) of the ITR. One example is a truncated AAV ITR that lacks a functional TRS. Without wishing to be bound by any particular theory, a rAAV vector comprising an ITR lacking a functional TRS produces a self-complementary rAAV vector, for example as described by McCarthy (2008) Molecular Therapy 16(10):1648-1656.

### Transduction Systems

As would be known to one of ordinary skill in the art, production of AAV in a cell (a packaging cell) can involve the introduction of more than one nucleic acid into the cell. For example, production of AAV can be accomplished by triple transfection. The most common approach to generate rAAV by transient transfection is a triple plasmid system in combination with E1-expressing cells (e.g., 293HEK). In such a system, one plasmid contains the vector genome, which includes a transgene sequence or an expression cassette (transgene sequence operably linked to a promoter) flanked by ITRs; the second plasmid (AAV helper) encodes Rep proteins and Cap proteins that are specific for the desired serotype (e.g., a subject retrograde-enhanced clade E variant AAV capsid protein); and the third plasmid (Ad helper) carries the minimal adenoviral genes required to support AAV replication (E2, E4 and VARNA). Notably, genes coding for AAV and Ad helper function can be cloned in a single plasmid and therefore a double (instead of triple) transfection approach can be sufficient to generate rAAV.

Thus, a subject transduction system includes one or more nucleic acids (e.g., 1, 2, or 3 nucleic acids, or in some cases more). In some cases, a subject transduction system includes from 1 to 3 nucleic acids. While all subject transduction systems include a nucleotide sequence that encodes a subject retrograde-enhanced clade E variant AAV capsid protein (e.g., AAV8-retro, rh10-retro) as a ‘first component’, such systems can include a ‘second component’ (e.g., a nucleotide sequence that encodes adenoviral genes to support AAV replication) and/or a ‘third component’ (e.g., transgene sequence or an expression cassette flanked by ITRs) as well—and the components can be included in any convenient combination.

For example, in some cases, a subject transduction system includes 1 nucleic acid that encodes a subject retrograde-enhanced clade E variant AAV capsid protein (e.g., AAV8-retro, rh10-retro). In some such cases, the one nucleic acid also encodes adenoviral genes to support AAV replication (e.g., E2, E4, and/or VARNA).

In some cases, a subject transduction system includes 2 nucleic acids. In such a system, the first nucleic acid encodes a subject retrograde-enhanced clade E variant AAV capsid protein (e.g., AAV8-retro, rh10-retro). In some cases, the first nucleic acid encodes a subject retrograde-enhanced clade E variant AAV capsid protein and the second nucleic acid encodes includes a transgene sequence or an expression cassette (transgene sequence operably linked to a promoter) flanked by ITRs. In some cases, the first nucleic acid encodes a subject retrograde-enhanced clade E variant AAV capsid protein and the second nucleic acid encodes adenoviral genes to support AAV replication. In some cases, the first nucleic acid encodes a subject retrograde-enhanced clade E variant AAV capsid protein and also encodes adenoviral genes to support AAV replication, and the second nucleic acid encodes includes a transgene sequence or an expression cassette (transgene sequence operably linked to a promoter) flanked by ITRs.

In some cases, a subject transduction system includes 3 nucleic acids. In such a system, the first nucleic acid encodes a subject retrograde-enhanced clade E variant AAV capsid protein (e.g., AAV8-retro, rh10-retro). In some cases, the second nucleic acid includes a transgene sequence or an expression cassette (transgene sequence operably linked to a promoter) flanked by ITRs. In some cases, the second nucleic acid encodes adenoviral genes to support AAV replication. In some cases, the second nucleic acid includes a transgene sequence or an expression cassette (transgene sequence operably linked to a promoter) flanked by ITRs, and the third nucleic acid encodes adenoviral genes to support AAV replication.

In light of the above, provided are transduction systems that include one or more nucleic acids, where the one or more nucleic acids encodes a retrograde-enhanced clade E variant AAV capsid protein (e.g., AAV8-retro, rh10-retro). In some cases, the one or more nucleic acids encodes a retrograde-enhanced clade E variant AAV capsid protein and also includes a transgene sequence or an expression cassette (transgene sequence operably linked to a promoter) flanked by ITRs. In some cases, the one or more nucleic acids encodes a retrograde-enhanced clade E variant AAV capsid protein and also encodes adenoviral genes to support AAV replication. In some cases, the one or more nucleic acids encodes a retrograde-enhanced clade E variant AAV capsid protein, includes a transgene sequence or an expression cassette (transgene sequence operably linked to a promoter) flanked by ITRs, and also encodes adenoviral genes to support AAV replication.

As noted, in some cases, the one or more nucleic acids include a transgene sequence, and in some cases the transgene sequence is present as part of an expression cassette, i.e., is operably linked to a promoter, e.g., a promoter that will function in the target cell to express the transgene sequence in the cell. In some cases the transgene sequence is not operably linked to a promoter (e.g., one may desire the transgene sequence to integrate into a target DNA in a target cell such that the transgene sequence will be under the control of a promoter, such as an endogenous promoter of the cell's genome, that is already present in the cell). The transgene sequence (or expression cassette) will in some cases be flanked by ITRs, as discussed in more detail elsewhere herein. The term “transgene sequence” encompasses any sequence of interest and a more detailed discussion of example transgene sequences is included elsewhere herein (See above)—any of which can be included in a subject transduction system. In some cases, a subject transduction system includes as a transgene sequence, an RNAi agent such as an shRNA (e.g., in some cases embedded in a miRNA), e.g., one that targets CamKv.

As such in some case as subject transduction system includes one or more nucleic acids, where the one or more nucleic acids encode a retrograde-enhanced clade E variant AAV capsid protein (e.g., AAV8-retro, rh10-retro), and also includes a transgene sequence (operably linked to a promoter or not) that encodes an shRNA that targets CamKv. I some cases the transgene sequenced encodes a genome-targeting effector protein such as a type II or type V CRISPR/Cas effector protein, a ZFN, or a TALEN.

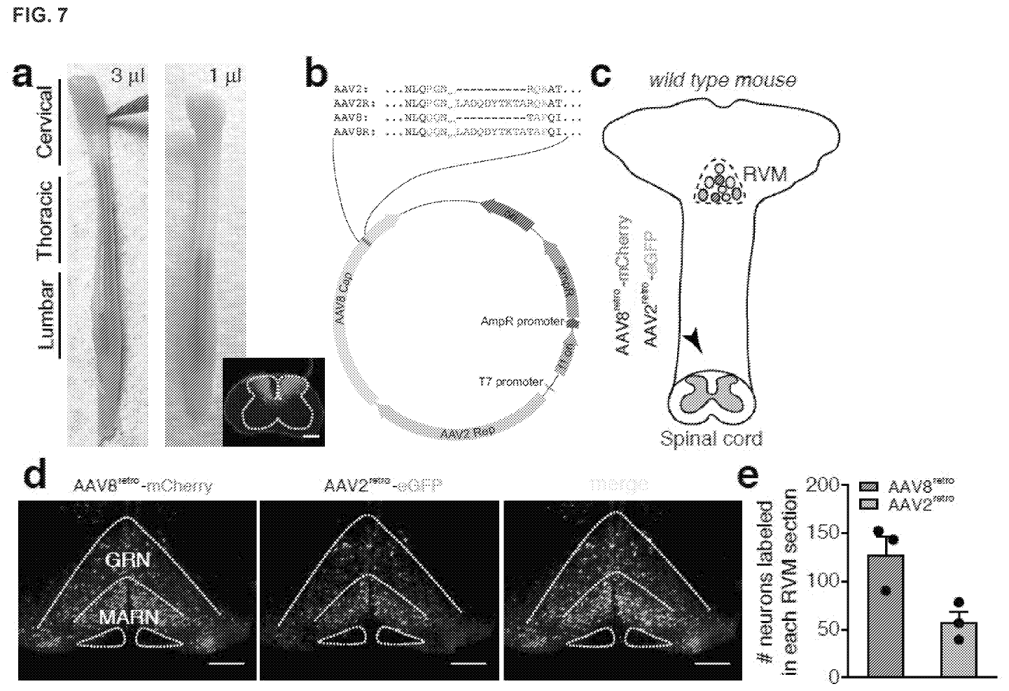

FIG. 7 (panel b) provides an illustrative example of a nucleic acid that includes a nucleotide sequence encoding a subject retrograde-enhanced clade E variant AAV capsid protein.

Aav Viral Particles and Methods of Making them

Methods of producing an AVV viral particle that includes a subject retrograde-enhanced clade E variant AAV capsid protein (e.g., AAV8-retro, rh10-retro) are provided. Such methods include introducing a transduction system (for example as discussed above) into a eukaryotic cell (e.g., a mammalian cell) that is competent for packaging AAV. Such cells will be known to one of ordinary skill in the art and any convenient competent cell can be used. For example, a subject method of making a retrograde-enhanced recombinant AAV particle includes introducing a nucleic acid that encodes a retrograde-enhanced clade E variant AAV capsid protein (e.g., AAV8-retro, rh10-retro) into a cell, and the cell produces the viral particle.

Retrograde-enhanced clade E variant AAV particles are also provided herein. Such a particle will include a clade E variant AAV capsid protein (e.g., AAV8-retro, rh10-retro). In some cases the particle will also include a nucleic acid that includes a transgene sequence or expression cassette (the transgene sequence operably linked to a promoter) flanked by ITRs. See the discussions elsewhere herein for details related to the transgene sequence and for details related to the ITRs. In some cases, a subject retrograde-enhanced clade E variant AAV includes a retrograde-enhanced clade E variant AAV capsid protein (e.g., AAV8-retro, rh10-retro) and also includes a transgene that encodes an RNAi agent such as an shRNA (e.g., in some cases embedded in a miRNA), e.g., one that targets CamKv. In some cases, the transgene encodes a genome-targeting effector protein such as a type II or type V CRISPR/Cas effector protein, a ZFN, or a TALEN. In some cases, the transgene encodes a marker protein (e.g., a fluorescent protein). In some cases, the transgene encodes a polypeptide intended to provide a therapeutic benefit.

### Host Cells and Packaging

Host cells can be used for generating infectious AAV vectors as well as for generating AAV virions based on the disclosed AAV vectors. Accordingly, the present disclosure provides host cells for generation and packaging of AAV virions based on the AAV vectors of the present disclosure. A variety of host cells are known in the art and find use in the methods of the present disclosure. Any host cells described herein or known in the art can be employed with the compositions and methods described herein.

The present disclosure provides host cells, e.g., comprising a subject rAAV particle (virion) and/or a subject nucleic acid. A subject host cell can be an isolated cell, e.g., a cell in in vitro culture. In some cases, the cell is in vivo. A subject host cell can be useful for producing a subject AAV vector or AAV virion, as described below. Where a subject host cell is used to produce a subject AAV virion, it is referred to as a “packaging cell.” In some embodiments, a subject host cell is stably genetically modified with a subject AAV vector. In other embodiments, a subject host cell is transiently genetically modified with a subject AAV vector.

In some embodiments, a subject nucleic acid is introduced stably or transiently into a host cell, using established techniques, including, but not limited to, electroporation, calcium phosphate precipitation, liposome-mediated transfection, baculovirus infection, and the like. For stable transformation, a subject nucleic acid will generally further include a selectable marker, e.g., any of several well-known selectable markers such as neomycin resistance, and the like.

In some embodiments, the host cell for use in generating infectious virions can be selected from any biological organism, including prokaryotic (e.g., bacterial) cells, and eukaryotic cells, including, insect cells, yeast cells and mammalian cells. A subject host cell is generated by introducing a subject nucleic acid (i.e., AAV vector) into any of a variety of cells, e.g., mammalian cells, including, e.g., murine cells, and primate cells (e.g., human cells). Particularly desirable host cells are selected from among any mammalian species. In some embodiments, cells include without limitation, cells such as A549, WEHI, 10T1/2, BHK, MDCK, COS 1, COS 7, BSC 1, BSC 40, BMT 10, WI38, HeLa, CHO, 293, Vero, NIH 3T3, PC12, Huh-7 Saos, C2C12, RAT1, Sf9, L cells, HT1080, human embryonic kidney (HEK), human embryonic stem cells, human adult tissue stem cells, pluripotent stem cells, induced pluripotent stem cells, reprogrammed stem cells, organoid stem cells, bone marrow stem cells, HLHepG2, HepG2 and primary fibroblast, hepatocyte and myoblast cells derived from mammals including human, monkey, mouse, rat, rabbit, and hamster. The selection of the mammalian species providing the cells is not a limitation of this disclosure; nor is the type of mammalian cell, i.e., fibroblast, hepatocyte, tumor cell, etc. The requirement for the cell used is it is capable of infection or transfection by an AAV vector. In some embodiments, the host cell is one that has Rep and Cap stably transfected in the cell, including in some embodiments a variant AAV capsid polypeptide as described herein. In some embodiments, the host cell expresses a variant AAV capsid polypeptide of the disclosure or part of an AAV vector as described herein, such as a heterologous nucleic acid sequence contained within the AAV vector.

In some embodiments, the preparation of a host cell according to the disclosure involves techniques such as assembly of selected DNA sequences. This assembly may be accomplished utilizing conventional techniques. Such techniques include cDNA and genomic cloning, which are well known and are described in Sambrook et al., cited above, use of overlapping oligonucleotide sequences of the adenovirus and AAV genomes, combined with polymerase chain reaction, synthetic methods, and any other suitable methods providing the desired nucleotide sequence.

In some embodiments, introduction of the AAV vector into the host cell may also be accomplished using techniques known to the skilled artisan and as discussed throughout the specification. In a preferred embodiment, standard transfection techniques are used, e.g., CaPO₄ transfection or electroporation, and/or infection by hybrid adenovirus/AAV vectors into cell lines such as the human embryonic kidney cell line HEK293 (a human kidney cell line containing functional adenovirus E1 genes providing trans-acting E1 proteins).

In some embodiments, a subject genetically modified host cell includes, in addition to a nucleic acid comprising a nucleotide sequence encoding a variant AAV capsid protein, as described above, a nucleic acid that comprises a nucleotide sequence encoding one or more AAV Rep proteins. In other embodiments, a subject host cell further comprises an AAV vector. An AAV virion can be generated using a subject host cell. Methods of generating an AAV virion are described in, e.g., U.S. Patent Publication No. 2005/0053922 and U.S. Patent Publication No. 2009/0202490.

In addition to an AAV vector, in some cases the host cell contains the sequences driving expression of the AAV capsid polypeptide (including variant AAV capsid polypeptides and non-variant parent capsid polypeptides) in the host cell and Rep sequences of the same serotype as the serotype of the AAV Inverted Terminal Repeats (ITRs) found in a nucleic acid that includes a transgene sequence, or a cross-complementing serotype. The AAV Cap and Rep sequences may be independently obtained from an AAV source and may be introduced into the host cell in any manner known to one of skill in the art or as described herein. Additionally, when pseudotyping an AAV vector in an AAV8 capsid for example, the sequences encoding each of the essential Rep proteins may be supplied by AAV8, or the sequences encoding the Rep proteins may be supplied by different AAV serotypes (e.g., AAV1, AAV2, AAV3, AAV4, AAV5, AAV6, AAV7, and/or AAV9).

In some embodiments, the host cell stably contains the capsid protein under the control of a suitable promoter (including, for example, the variant AAV capsid polypeptides of the disclosure), such as those described above. In some embodiments, the capsid protein is expressed under the control of an inducible promoter. In some embodiments, the capsid protein is supplied to the host cell in trans. When delivered to the host cell in trans, the capsid protein may be delivered via a plasmid containing the sequences necessary to direct expression of the selected capsid protein in the host cell. In some embodiments, when delivered to the host cell in trans, the vector encoding the capsid protein (including, for example, the variant AAV capsid polypeptides of the disclosure) also carries other sequences required for packaging the AAV, e.g., the Rep sequences.

In some embodiments, the host cell stably contains the Rep sequences under the control of a suitable promoter, such as those described above. In some embodiments, the essential Rep proteins are expressed under the control of an inducible promoter. In another embodiment, the Rep proteins are supplied to the host cell in trans. When delivered to the host cell in trans, the Rep proteins may be delivered via a plasmid containing the sequences necessary to direct expression of the selected Rep proteins in the host cell. In some embodiments, when delivered to the host cell in trans, the vector encoding the capsid protein (including, for example, the variant AAV capsid polypeptides of the disclosure) also carries other sequences required for packaging the AAV vector, e.g., the Rep sequences.

In some embodiments, the Rep and Cap sequences may be transfected into the host cell on a single nucleic acid molecule and exist stably in the cell as an unintegrated episome. In another embodiment, the Rep and Cap sequences are stably integrated into the chromosome of the cell. Another embodiment has the Rep and Cap sequences transiently expressed in the host cell. For example, a useful nucleic acid molecule for such transfection comprises, from 5′ to 3′, a promoter, an optional spacer interposed between the promoter and the start site of the Rep gene sequence, an AAV Rep gene sequence, and an AAV Cap gene sequence.

Although the molecule(s) providing Rep and capsid can exist in the host cell transiently (i.e., through transfection), in some embodiments, one or both of the Rep and capsid proteins and the promoter(s) controlling their expression be stably expressed in the host cell, e.g., as an episome or by integration into the chromosome of the host cell. The methods employed for constructing embodiments of the disclosure are conventional genetic engineering or recombinant engineering techniques such as those described in the references above.

In some embodiments, the packaging host cell can require helper functions in order to package the AAV vector of the disclosure into an AAV virion. In some embodiments, these functions may be supplied by a herpesvirus. In some embodiments, the necessary helper functions are each provided from a human or non-human primate adenovirus source, and are available from a variety of sources, including the American Type Culture Collection (ATCC), Manassas, Va. (US). In some embodiments, the host cell is provided with and/or contains an E1a gene product, an E1b gene product, an E2a gene product, and/or an E4 ORF6 gene product. In some embodiments, the host cell may contain other adenoviral genes such as VAI RNA. In some embodiments, no other adenovirus genes or gene functions are present in the host cell.

### Methods of Generating an AAV Virion

In various embodiments, the disclosure provides a method for generating an AAV virion of the disclosure. A variety of methods for generating AAV virions are known in the art and can be used to generate AAV virions comprising the AAV vectors described herein. Generally, the methods involve inserting or transducing an AAV vector of the disclosure into a host cell capable of packaging the AAV vector into an AAV virion. Exemplary methods are described and referenced below; however, any method known to one of skill in the art can be employed to generate the AAV virions of the disclosure.

An AAV vector comprising a heterologous nucleic acid and used to generate an AAV virion can be constructed using any convenient method, including methods that are well known in the art. See, e.g., Koerber et al. (2009) Mol. Ther., 17:2088; Koerber et al. (2008) Mol Ther., 16: 1703-1709; as well as U.S. Pat. Nos. 7,439,065, 6,951,758, and 6,491,907. For example, the heterologous sequence(s) can be directly inserted into an AAV genome with the major AAV open reading frames (“ORFs”) excised therefrom. Other portions of the AAV genome can also be deleted, so long as a sufficient portion of the ITRs remain to allow for replication and packaging functions. Such constructs can be designed using techniques well known in the art. See, e.g., U.S. Pat. Nos. 5,173,414 and 5,139,941; International Publication Nos. WO 92/01070 (published Jan. 23, 1992) and WO 93/03769 (published Mar. 4, 1993); Lebkowski et al. (1988) Molec. Cell. Biol. 8:3988-3996; Vincent et al. (1990) Vaccines 90 (Cold Spring Harbor Laboratory Press); Carter, B. J. (1992) Current Opinion in Biotechnology 3:533-539; Muzyczka, N. (1992) Curr. Topics Microbiol. Immunol. 158:97-129; Kotin, R. M. (1994) Human Gene Therapy 5:793-801; Shelling and Smith (1994) Gene Therapy 1:165-169; and Zhou et al. (1994) J. Exp. Med. 179:1867-1875.

In order to produce AAV virions, an AAV vector can be introduced into a suitable host cell using known techniques, such as by transfection. A number of transfection techniques are generally known in the art. See, e.g., Graham et al. (1973) Virology, 52:456, Sambrook et al. (1989) Molecular Cloning, A Laboratory Manual, Cold Spring Harbor Laboratories, New York, Davis et al. (1986) Basic Methods in Molecular Biology, Elsevier, and Chu et al. (1981) Gene 13:197. Particularly suitable transfection methods include calcium phosphate co-precipitation (Graham et al. (1973) Virol. 52:456-467), direct micro-injection into cultured cells (Capecchi, M. R. (1980) Cell 22:479-488), electroporation (Shigekawa et al. (1988) BioTechniques 6:742-751), liposome-mediated gene transfer (Mannino et al. (1988) BioTechniques 6:682-690), lipid-mediated transduction (Feigner et al. (1987) Proc. Natl. Acad. Sci. USA 84:7413-7417), and nucleic acid delivery using high-velocity microprojectiles (Klein et al. (1987) Nature 327:70-73).

Suitable host cells for producing AAV virions include any species and/or type of cell that can be, or have been, used as recipients of a heterologous AAV DNA molecule, and can support the expression of required AAV production cofactors from helper viruses. Such host cells can include but are not limited to microorganisms, yeast cells, insect cells, and mammalian cells, that can be, or have been, used as recipients of a heterologous DNA molecule. The term includes the progeny of the original cell transfected. Thus, a “host cell” as used herein generally refers to a cell transfected with an exogenous DNA sequence. Cells from the stable human cell line, HEK293 (readily available through, e.g., the American Type Culture Collection under Accession Number ATCC CRL1573) can be used. The human cell line HEK293 is a human embryonic kidney cell line that has been transformed with adenovirus type-5 DNA fragments (Graham et al. (1977) J. Gen. Virol. 36:59), and expresses the adenoviral E1a and E1b genes (Aiello et al. (1979) Virology 94:460). The HEK293 cell line is readily transfected, and provides a convenient platform in which to produce AAV virions.

Methods of producing an AAV virion in insect cells are known in the art, and can be used to produce a subject AAV virion. See, e.g., U.S. Patent Publication No. 2009/0203071; U.S. Pat. No. 7,271,002; and Chen (2008) Mol. Ther. 16:924.

In some embodiments, the AAV virion or AAV vector is packaged into an infectious virion or virus particle, by any of the methods described herein or known in the art.

In some embodiments, the variant AAV capsid polypeptide allows for similar packaging as compared to a non-variant parent capsid polypeptide. In some embodiments, an AAV vector packaged with the variant AAV capsid polypeptides transduce into cells in vivo better than a vector packaged from non-variant parent capsid polypeptides. In some embodiments, the AAV vector packaged with the variant AAV capsid polypeptides transduce into cells in vitro better than a vector packaged from non-variant parent capsid polypeptides. In some embodiments, the variant AAV capsid polypeptides result in nucleic acid expression higher than a nucleic acid packaged from non-variant parent capsid polypeptides. In some embodiments, the AAV vector packaged with said variant AAV capsid polypeptides result in transgene expression better than a transgene packaged from non-variant parent capsid polypeptides.

### Pharmaceutical Compostions & Dosing

Also provided are pharmaceutical compositions useful in treating subjects according to the methods of the disclosure as described herein. Further, the present disclosure provides dosing regimens for administering the described pharmaceutical compositions. The present disclosure provides pharmaceutical compositions comprising: a) a subject AAV vector or AAV virion, as described herein as well as therapeutic molecules packaged by or within capsids comprising variant polypeptides as described herein; and b) a pharmaceutically acceptable carrier, diluent, excipient, or buffer. In some embodiments, the pharmaceutically acceptable carrier, diluent, excipient, or buffer is suitable for use in a human.

Such excipients, carriers, diluents, and buffers include any pharmaceutical agent that can be administered without undue toxicity. Pharmaceutically acceptable excipients include, but are not limited to, liquids such as water, saline, glycerol and ethanol. Pharmaceutically acceptable salts can be included therein, for example, mineral acid salts such as hydrochlorides, hydrobromides, phosphates, sulfates, and the like; and the salts of organic acids such as acetates, propionates, malonates, benzoates, and the like. Additionally, auxiliary substances, such as wetting or emulsifying agents, pH buffering substances, and the like, may be present in such vehicles. A wide variety of pharmaceutically acceptable excipients are known in the art and need not be discussed in detail herein. Pharmaceutically acceptable excipients have been amply described in a variety of publications, including, for example, A. Gennaro, (2000) Remington: The Science and Practice of Pharmacy, 20th edition, Lippincott, Williams, & Wilkins; Pharmaceutical Dosage Forms and Drug Delivery Systems (1999) H. C. Ansel et al., eds., 7.sup.th ed., Lippincott, Williams, & Wilkins; and Handbook of Pharmaceutical Excipients (2000) A. H. Kibbe et al., eds., 3.sup.rd ed. Amer. Pharmaceutical Assoc.

A subject composition can comprise a liquid comprising a subject variant AAV capsid polypeptide of the disclosure or AAV virion comprising a variant AAV capsid polypeptide in solution, in suspension, or both. As used herein, liquid compositions include gels. In some cases, the liquid composition is aqueous. In some embodiments, the composition is an in situ gellable aqueous composition, e.g., an in situ gellable aqueous solution. Aqueous compositions have ophthalmically compatible pH and osmolality.

Such compositions include solvents (aqueous or non-aqueous), solutions (aqueous or non-aqueous), emulsions (e.g., oil-in-water or water-in-oil), suspensions, syrups, elixirs, dispersion and suspension media, coatings, isotonic and absorption promoting or delaying agents, compatible with pharmaceutical administration or in vivo contact or delivery. Aqueous and non-aqueous solvents, solutions and suspensions may include suspending agents and thickening agents. Such pharmaceutically acceptable carriers include tablets (coated or uncoated), capsules (hard or soft), microbeads, powder, granules and crystals. Supplementary active compounds (e.g., preservatives, antibacterial, antiviral and antifungal agents) can also be incorporated into the compositions.

Pharmaceutical compositions can be formulated to be compatible with a particular route of administration or delivery, as set forth herein or known to one of skill in the art. Thus, pharmaceutical compositions include carriers, diluents, or excipients suitable for administration by various routes.

Compositions suitable for parenteral administration comprise aqueous and non-aqueous solutions, suspensions or emulsions of the active compound. Preparations are typically sterile and can be isotonic with the blood of the intended recipient. Non-limiting illustrative examples include water, saline, dextrose, fructose, ethanol, animal, vegetable or synthetic oils.

For transmucosal or transdermal administration (e.g., topical contact), penetrants can be included in the pharmaceutical composition. Penetrants are known in the art, and include, for example, for transmucosal administration, detergents, bile salts, and fusidic acid derivatives. For transdermal administration, the active ingredient can be formulated into aerosols, sprays, ointments, salves, gels, or creams as generally known in the art. For contact with skin, pharmaceutical compositions typically include ointments, creams, lotions, pastes, gels, sprays, aerosols, or oils. Useful carriers include Vaseline®, lanolin, polyethylene glycols, alcohols, transdermal enhancers, and combinations thereof.

Cosolvents and adjuvants may be added to the formulation. Non-limiting examples of cosolvents contain hydroxyl groups or other polar groups, for example, alcohols, such as isopropyl alcohol; glycols, such as propylene glycol, polyethyleneglycol, polypropylene glycol, glycol ether; glycerol; polyoxyethylene alcohols and polyoxyethylene fatty acid esters. Adjuvants include, for example, surfactants such as, soya lecithin and oleic acid; sorbitan esters such as sorbitan trioleate; and polyvinylpyrrolidone.

Pharmaceutical compositions and delivery systems appropriate for the AAV vector or AAV virion and methods and uses of are known in the art (see, e.g., Remington: The Science and Practice of Pharmacy (2003) 20.sup.th ed., Mack Publishing Co., Easton, Pa.; Remington's Pharmaceutical Sciences (1990) 18.sup.th ed., Mack Publishing Co., Easton, Pa.; The Merck Index (1996) 12.sup.th ed., Merck Publishing Group, Whitehouse, N.J.; Pharmaceutical Principles of Solid Dosage Forms (1993), Technonic Publishing Co., Inc., Lancaster, Pa.; Ansel and Stoklosa, Pharmaceutical Calculations (2001) 11.sup.th ed., Lippincott Williams & Wilkins, Baltimore, Md.; and Poznansky et al., Drug Delivery Systems (1980), R. L. Juliano, ed., Oxford, N.Y., pp. 253-315).

Doses can vary and depend upon whether the treatment is prophylactic or therapeutic, the type, onset, progression, severity, frequency, duration, or probability of the disease treatment is directed to, the clinical endpoint desired, previous or simultaneous treatments, the general health, age, gender, race or immunological competency of the subject and other factors that will be appreciated by the skilled artisan. The dose amount, number, frequency or duration may be proportionally increased or reduced, as indicated by any adverse side effects, complications or other risk factors of the treatment or therapy and the status of the subject. The skilled artisan will appreciate the factors that may influence the dosage and timing required to provide an amount sufficient for providing a therapeutic or prophylactic benefit.

Methods and uses of the disclosure as disclosed herein can be practiced within about 1 hour to about 2 hours, about 2 hours to about 4 hours, about 4 hours to about 12 hours, about 12 hours to about 24 hours or about 24 hours to about 72 hours after a subject has been identified as having the disease targeted for treatment, has one or more symptoms of the disease, or has been screened and is identified as positive as set forth herein even though the subject does not have one or more symptoms of the disease. In some embodiments, the disclosure as disclosed herein can be practiced within about 1 hour, about 2 hours, about 3 hours, about 4 hours, about 5 hours, about 6 hours, about 7 hours, about 8 hours, about 9 hours, about 10 hours, about 11 hours, about 12 hours, about 24 hours, about 36 hours, about 48 hours, or about 72 hours or more. Of course, methods and uses of the disclosure can be practiced about 1 day to about 7 days, about 7 days to about 14 days, about 14 days to about 21 days, about 21 days to about 48 days or more, months or years after a subject has been identified as having the disease targeted for treatment, has one or more symptoms of the disease, or has been screened and is identified as positive as set forth herein. In some embodiments, the disclosure as disclosed herein can be practiced within about 1 day, about 2 days, about 3 days, about 4 days, about 5 days, about 6 days, about 7 days, about 8 days, about 9 days, about 10 days, about 11 days, about 12 days, about 14 days, about 21 days, about 36 days, or about 48 days or more.

In some embodiments, the present disclosure provides kits with packaging material and one or more components therein. A kit typically includes a label or packaging insert including a description of the components or instructions for use in vitro, in vivo, or ex vivo, of the components therein. A kit can contain a collection of such components, e.g., a variant AAV capsid polypeptide, an AAV vector, a nucleic acid encoding a variant AAV protein, and/or an AAV virion (in any combination thereof) and optionally a second active ingredient, such as another compound, agent, drug or composition.

A kit refers to a physical structure housing one or more components of the kit. Packaging material can maintain the components sterilely, and can be made of material commonly used for such purposes (e.g., paper, corrugated fiber, glass, plastic, foil, ampules, vials, tubes, etc.).

Labels or inserts can include identifying information of one or more components therein, dose amounts, clinical pharmacology of the active ingredient(s) including mechanism of action, pharmacokinetics and pharmacodynamics. Labels or inserts can include information identifying the manufacturer, lot numbers, manufacturer location and date, expiration dates. Labels or inserts can include information identifying manufacturer information, lot numbers, manufacturer location and date. Labels or inserts can include information on a disease a kit component may be used for. Labels or inserts can include instructions for the clinician or subject for using one or more of the kit components in a method, use, or treatment protocol or therapeutic regimen. Instructions can include dosage amounts, frequency or duration, and instructions for practicing any of the methods, uses, treatment protocols or prophylactic or therapeutic regimes described herein.

Labels or inserts can include information on any benefit that a component may provide, such as a prophylactic or therapeutic benefit. Labels or inserts can include information on potential adverse side effects, complications or reactions, such as warnings to the subject or clinician regarding situations where it would not be appropriate to use a particular composition. Adverse side effects or complications could also occur when the subject has, will be or is currently taking one or more other medications that may be incompatible with the composition, or the subject has, will be or is currently undergoing another incompatible treatment protocol or therapeutic regimen and, therefore, instructions could include information regarding such incompatibilities.

Labels or inserts include “printed matter,” e.g., paper or cardboard, or separate or affixed to a component, a kit or packing material (e.g., a box), or attached to an ampule, tube or vial containing a kit component. Labels or inserts can additionally include a computer readable medium, such as a bar-coded printed label, a disk, optical disk such as CD- or DVD-ROM/RAM, DVD, MP3, magnetic tape, or an electrical storage media such as RAM and ROM or hybrids of these such as magnetic/optical storage media, FLASH media or memory type cards.

### Methods of Expressing a Transgene in Neuron

The present disclosure provides methods of expressing a transgene in a neuron—in some cases to a neuron of an individual. In some cases the neuron is in vitro, e.g., is a neuron in culture, and in some cases the neuron is in vivo, e.g., is in the body of an individual. In some cases, a method of treatment, e.g., to treat chronic pain, does not include the use of a subject retrograde-enhanced clade E variant AAV (having a retrograde-enhanced clade E variant AAV capsid protein) as described herein. In other cases, a method of treatment, e.g., to treat chronic pain, does include the use of a subject retrograde-enhanced clade E variant AAV (having a retrograde-enhanced clade E variant AAV capsid protein) as described herein—and therefore in some cases utilizes a subject method of expressing a transgene.

The present disclosure provides methods for expressing a transgene of interest in a neuron. Such method can include a step of contacting a neuron with a subject retrograde-enhanced clade E variant AAV (e.g., AAV8-retro, rh10-retro, and the like—see detailed description of such AAVs elsewhere herein). In some cases the neuron is in vitro (e.g., in cell culture, part of a tissue sample such as a brain slice, and the like). In some cases the neuron is in vivo (i.e., in an individual's body)—and as such contacting the neuron can be accomplished by administering a subject retrograde-enhanced clade E variant AAV (see more detailed description elsewhere herein) to an individual.

Contacting can occur in any convenient location. For example, in some cases, the contacting occurs in an individual's spinal cord (e.g., via injection). In some cases, the contacting occurs in an individual's thalamus. The neuron can be any desired neuron. For example, in some cases, the neuron is a projection neuron. Projection neurons are neurons whose axons extend from a neuronal cell body within the central nervous system (CNS) to one or more distant regions of the CNS. Examples of such include, but are not limited to rostral ventromedial medulla (RVM), thalamocortical, corticothalamic, zona incerta, and lateral superior colliculus (lSCIndG) projection neurons. In some cases, the neuron is a spinal cord projecting neuron. In some cases, the neuron is a spinal cord-projecting neuron of the rostral ventromedial medulla (RVM). In some cases, the neuron is a spinal cord-projecting neuron is a neuron of the locus coeruleus (LC). In some cases, the neuron is a corticothalamic projecting neuron. In some cases, the neuron is an opioid receptor mu 1 (OPRM1) expressing neuron (e.g., in some cases one that is a spinal cord-projecting neuron of the RVM).

In some cases the retrograde-enhanced clade E variant AAV will encapsidate a nucleic acid that includes a transgene sequence. Any desirable transgene sequence can be used (see detailed discussion of transgene sequences elsewhere herein). For example, in some cases the transgene sequence encodes a non-coding RNA such as an RNAi agent or a CRISPR/Cas guide RNA. In some cases the transgene sequence encodes a protein (e.g., a genome-targeting protein, a therapeutic protein, and the like). In some cases an RNAi agent targets CamKv. In some cases a guide RNA targets CamKv. In some cases a genome-targeting protein is a CRISPR/Cas effector protein that is nuclease defect and is associate with (e.g., fused to) a transcriptional repressor domain (e.g., a chromatin modifying domain, a DNA-modification domain, and the like) (e.g., CRISPRi/CRISPRa).

In an example embodiment, the disclosure provides a method of administering a pharmaceutical composition of the disclosure to a subject in need thereof to treat a disease or disorder of a subject (e.g., chronic pain).

In some embodiments, the subject variant AAV capsid polypeptide (retrograde-enhanced clade E variant adeno-associated virus (AAV) capsid protein, e.g., AAV8-retro or rh10-retro) packages a therapeutic expression cassette comprised of a heterologous nucleic acid comprising a nucleotide sequence encoding a heterologous gene product, such as for example a therapeutic protein. In some embodiments, the AAV virion or AAV vector comprises a therapeutic expression cassette comprised of a heterologous nucleic acid comprising a nucleotide sequence encoding a non-coding RNA such as a guide RNA or an RNAi agent. In some such cases the non-coding RNA targets CamKv.

In some embodiments, the variant AAV capsid polypeptides of the disclosure are employed as part of vaccine delivery. Vaccine delivery can include delivery of any of the therapeutic proteins as well as nucleic acids described herein. In some embodiments, variant AAV capsid polypeptides of the disclosure are employed as part of a vaccine regimen and dosed according to the methods described herein.

In some embodiments, the variant AAV capsid polypeptides, the AAV virions, or AAV vectors of the disclosure are used in a therapeutic treatment regimen.

In some embodiments, the variant AAV capsid polypeptides, the AAV virions, or AAV vectors of the disclosure are used for therapeutic polypeptide production.

In some cases, a subject variant AAV capsid polypeptides or AAV vector, when introduced into the cells of a subject, provides for high level production of the heterologous gene product packaged by the variant AAV capsid polypeptides or encoded by the AAV vector. For example, a heterologous polypeptide packaged by the variant AAV capsid polypeptides or encoded by the AAV can be produced.

In some cases, subject retrograde-enhanced clade E variant AAV capsid polypeptides, AAV virion, or AAV vector, when introduced into a subject, provide for production of the heterologous gene product packaged by the variant AAV capsid polypeptides or encoded by the AAV vector in at least about 10%, at least about 15%, at least about 20%, at least about 25%, at least about 30%, at least about 35%, at least about 40%, at least about 50% at least about 60%, at least about 70%, at least about 80%, or more than 80%, of the target cells.

In some embodiments, the present disclosure provides a method of treating a disease or disorder (e.g., chronic pain), the method comprising administering to an individual in need thereof an effective amount of a therapeutic molecule packaged by the variant AAV capsid polypeptides or subject AAV vector (e.g., a transgene sequence encoding a non-coding RNA or a protein) as described elsewhere herein.

Subject retrograde-enhanced clade E variant AAV can be administered systemically, regionally or locally, or by any route, for example, by injection, infusion, orally (e.g., ingestion or inhalation), or topically (e.g., transdermally). Possible delivery and administration methods can include parenteral, intravenous, intramuscular, intraperitoneal, intradermal, subcutaneous, intracavity, intracranial, transdermal (topical), transmucosal and rectal administration. Example administration and delivery routes include intravenous, intraperitoneal, intrarterial, parenteral, subcutaneous, intra-pleural, topical, dermal, intradermal, transdermal, transmucosal, oral (alimentary), mucosal, respiration, intranasal, intubation, intrapulmonary, intrapulmonary instillation, buccal, sublingual, intravascular, intrathecal, intracavity, iontophoretic, intraocular, ophthalmic, optical, intraglandular, intraorgan, and intralymphatic. In some cases the delivery route is systemic (e.g., parenteral, intravenous).

In some cases, a therapeutically effective amount of a therapeutic molecule packaged by the variant AAV capsid polypeptides or a subject AAV vectors is an amount that, when administered to an individual in one or more doses, is effective to slow the progression of the disease or disorder in the individual, or is effective to ameliorate symptoms. For example, a therapeutically effective amount of a therapeutic molecule (e.g., an RNAi agent) packaged by the variant AAV capsid polypeptides or a subject AAV vectors can be an amount that, when administered to an individual in one or more doses, is effective to slow the progression of the disease or disorder (e.g., chronic pain) by at least about 15%, at least about 20%, at least about 25%, at least about 30%, at least about 40%, at least about 50%, at least about 60%, at least about 70%, at least about 80%, or more than about 80%, compared to the progression of the disease in the absence of treatment with the therapeutic molecule packaged by the variant AAV capsid polypeptides or AAV vectors.

A therapeutic or beneficial effect of treatment is therefore any objective or subjective measurable or detectable improvement or benefit provided to a particular subject. A therapeutic or beneficial effect can but need not be complete ablation of all or any particular adverse symptom, disorder, illness, or complication of a disease. Thus, a satisfactory clinical endpoint is achieved when there is an incremental improvement or a partial reduction in an adverse symptom, disorder, illness, or complication caused by or associated with a disease, or an inhibition, decrease, reduction, suppression, prevention, limit or control of worsening or progression of one or more adverse symptoms, disorders, illnesses, or complications caused by or associated with the disease, over a short or long duration (hours, days, weeks, months, etc.).

Improvement of clinical symptoms can also be monitored by one or more methods known to the art, and used as an indication of therapeutic effectiveness. Clinical symptoms may also be monitored by anatomical or physiological means. In some embodiments, a therapeutic molecule (including, for example, nucleic acid that includes a nucleotide sequence of interest) packaged by the variant AAV capsid polypeptides, a subject AAV vector, or AAV virus, when introduced into a subject, provides for production of a heterologous gene product (e.g., non-coding or coding RNA, a protein) for a period of time from about 2 days to about 6 months, e.g., from about 2 days to about 7 days, from about 1 week to about 4 weeks, from about 1 month to about 2 months, or from about 2 months to about 6 months. In some embodiments, therapeutic molecules packaged by the variant AAV capsid polypeptides, a subject AAV vector or virus, when introduced into a subject provides for production of the heterologous gene product for a period of time of more than 6 months, e.g., from about 6 months to 20 years or more, or greater than 1 year, e.g., from about 6 months to about 1 year, from about 1 year to about 2 years, from about 2 years to about 5 years, from about 5 years to about 10 years, from about 10 years to about 15 years, from about 15 years to about 20 years, or more than 20 years.

Multiple doses of a subject AAV virion can be administered to an individual in need thereof. Where multiple doses are administered over a period of time, an active agent is administered once a month to about once a year, from about once a year to once every 2 years, from about once every 2 years to once every 5 years, or from about once every 5 years to about once every 10 years, over a period of time. For example, a subject AAV virion is administered over a period of from about 3 months to about 2 years, from about 2 years to about 5 years, from about 5 years to about 10 years, from about 10 years to about 20 years, or more than 20 years. The actual frequency of administration, and the actual duration of treatment, depends on various factors. In some embodiments, the administration regimen is part of a vaccination regimen.

The dose to achieve a therapeutic effect, e.g., the dose in vector genomes/per kilogram of body weight (vg/kg), will vary based on several factors including, but not limited to: route of administration, the level of heterologous polynucleotide expression required to achieve a therapeutic effect, the specific disease treated, any host immune response to the viral vector, a host immune response to the heterologous polynucleotide or expression product (e.g., RNA or protein), and the stability of the expressed molecule. One skilled in the art can readily determine a virion dose range to treat a patient having a particular disease or disorder based on the aforementioned factors, as well as other factors. In some embodiments, doses will range from at least about, or more, for example, 1×10⁹, 1×10¹⁰, 1×10¹¹, 1×10¹², 1×10¹³, or 1×10¹⁴, or more, vector genomes per kilogram (vg/kg) of the weight of the subject, to achieve a therapeutic effect.

An effective amount or a sufficient amount can, but need not be, provided in a single administration, may require multiple administrations, and, can but need not be, administered alone or in combination with another composition (e.g., agent), treatment, protocol or therapeutic regimen. For example, the amount may be proportionally increased as indicated by the need of the subject, type, status and severity of the disease treated or side effects (if any) of treatment. In addition, an effective amount or a sufficient amount need not be effective or sufficient if given in single or multiple doses without a second composition (e.g., another drug or agent), treatment, protocol or therapeutic regimen, since additional doses, amounts or duration above and beyond such doses, or additional compositions (e.g., drugs or agents), treatments, protocols or therapeutic regimens may be included in order to be considered effective or sufficient in a given subject. Amounts considered effective also include amounts that result in a reduction of the use of another treatment, therapeutic regimen or protocol.

An effective amount or a sufficient amount need not be effective in each and every subject treated, or a majority of treated subjects in a given group or population. An effective amount or a sufficient amount means effectiveness or sufficiency in a particular subject, not a group or the general population. As is typical for such methods, some subjects will exhibit a greater response, or less or no response to a given treatment method or use. Thus, appropriate amounts will depend upon the condition treated, the therapeutic effect desired, as well as the individual subject (e.g., the bioavailability within the subject, gender, age, etc.).

With regard to a disease or disorder or symptom thereof, or an underlying cellular response, a detectable or measurable improvement includes a subjective or objective decrease, reduction, inhibition, suppression, limit or control in the occurrence, frequency, severity, progression, or duration of the disease or disorder, or complication caused by or associated with the disease or disorder, or an improvement in a symptom or an underlying cause or a consequence of the disease or disorder, or a reversal of the disease or disorder.

Thus, a successful treatment outcome can lead to a “therapeutic effect,” or “benefit” of decreasing, reducing, inhibiting, suppressing, limiting, controlling or preventing the occurrence, frequency, severity, progression, or duration of a disease or disorder or disorder, or one or more adverse symptoms or underlying causes or consequences of the disease or disorder in a subject. Treatment methods and uses affecting one or more underlying causes of the disease or disorder or adverse symptoms are therefore considered to be beneficial. A decrease or reduction in worsening, such as stabilizing the disease or disorder, or an adverse symptom thereof, is also a successful treatment outcome.

A therapeutic benefit or improvement therefore need not be complete ablation of the disease or disorder, or any one, most or all adverse symptoms, complications, consequences or underlying causes associated with the disease or disorder. Thus, a satisfactory endpoint is achieved when there is an incremental improvement in a subject's disease or disorder, or a partial decrease, reduction, inhibition, suppression, limit, control or prevention in the occurrence, frequency, severity, progression, or duration, or inhibition or reversal, of the disease or disorder (e.g., stabilizing one or more symptoms or complications), over a short or long duration of time (hours, days, weeks, months, etc.). Effectiveness of a method or use, such as a treatment that provides a potential therapeutic benefit or improvement of a disease or disorder, can be ascertained by various methods.

Disclosed methods and uses can be combined with any compound, agent, drug, treatment or other therapeutic regimen or protocol having a desired therapeutic, beneficial, additive, synergistic or complementary activity or effect. Exemplary combination compositions and treatments include second actives, such as, biologics (proteins), agents and drugs. Such biologics (proteins), agents, drugs, treatments and therapies can be administered or performed prior to, substantially contemporaneously with or following any other method or use of the disclosure.

The compound, agent, drug, treatment or other therapeutic regimen or protocol can be administered as a combination composition, or administered separately, such as concurrently or in series or sequentially (prior to or following) delivery or administration of an AAV vector or AAV virion as described herein. The disclosure therefore provides combinations where a method or use of the disclosure is in a combination with any compound, agent, drug, therapeutic regimen, treatment protocol, process, remedy or composition, set forth herein or known to one of skill in the art. The compound, agent, drug, therapeutic regimen, treatment protocol, process, remedy or composition can be administered or performed prior to, substantially contemporaneously with or following administration of an AAV vector or AAV virion as described herein, to a subject. Specific non-limiting examples of combination embodiments therefore include the foregoing or other compound, agent, drug, therapeutic regimen, treatment protocol, process, remedy or composition.

Methods and uses of the disclosure also include, among other things, methods and uses that result in a reduced need or use of another compound, agent, drug, therapeutic regimen, treatment protocol, process, or remedy. For example, for chronic pain, a method or use of the disclosure has a therapeutic benefit if in a given subject a less frequent or reduced dose or elimination of administration of an anti-pain medication in the subject. Thus, in accordance with the disclosure, methods and uses of reducing need or use of another treatment or therapy are provided.

The disclosure is useful in animals including veterinary medical applications. Suitable subjects therefore include mammals, such as humans, as well as non-human mammals such as non-human primates. The term “subject” refers to an animal, typically a mammal, such as humans, non-human primates (apes, gibbons, gorillas, chimpanzees, orangutans, macaques), a domestic animal (dogs and cats), a farm animal (poultry such as chickens and ducks, horses, cows, goats, sheep, pigs), and experimental animals (mouse, rat, rabbit, guinea pig). Human subjects include fetal, neonatal, infant, juvenile and adult subjects. Subjects include animal disease models, for example, mouse and other animal models of blood clotting diseases and others known to those of skill in the art.

In some embodiments, a method or use of the disclosure includes: (a) providing an AAV virion whose capsid comprises a variant AAV capsid polypeptide (e.g., prepared as described herein), wherein the AAV virion comprises a heterologous nucleic acid sequence (e.g., in some cases operably linked to an expression control element conferring transcription of said nucleic acid sequence); and (b) administering an amount of the AAV virion to the mammal such that said heterologous nucleic acid is expressed in the mammal.

In some embodiments, a method or use of the disclosure includes: (a) providing a therapeutic molecule packaged by variant AAV capsid polypeptides (e.g., prepared as described herein), wherein the therapeutic molecule comprises a heterologous nucleic acid sequence (e.g., which can in some cases be operably linked to an expression control element conferring transcription of said nucleic acid sequence); and (b) administering an amount of the therapeutic molecule (including, for example, a vaccine) packaged by variant AAV capsid polypeptides to the mammal such that said heterologous nucleic acid is expressed in the mammal.

In some embodiments, a method or use of the disclosure includes delivering or transferring a heterologous polynucleotide sequence into a mammal or a cell of a mammal, by administering a heterologous polynucleotide packaged by the variant AAV capsid polypeptides, a plurality of heterologous polynucleotides packaged by variant AAV capsid polypeptides, an AAV virion prepared as described herein, or a plurality of AAV virions comprising the heterologous nucleic acid sequence to a mammal or a cell of a mammal, thereby delivering or transferring the heterologous polynucleotide sequence into the mammal or cell of the mammal. In some embodiments, the heterologous nucleic acid sequence encodes a protein expressed in the mammal, or where the heterologous nucleic acid sequence encodes an inhibitory sequence or protein that reduces expression of an endogenous protein in the mammal.

In some embodiments, a method or use of the disclosure includes is a method of expressing a transgene of interest to the central nervous system of an individual, and includes administering to the individual a nucleic acid or a recombinant AAV (rAAV) particle as described herein.

### Additional Definitions

Unless defined otherwise, all technical and scientific terms used herein have the same meaning as commonly understood by one of ordinary skill in the art to which the invention pertains. In describing and claiming the present invention, the following terminology will be used.

The term “abnormal” when used in the context of organisms, tissues, cells or components thereof, refers to those organisms, tissues, cells or components thereof that differ in at least one observable or detectable characteristic (e.g., age, treatment, time of day, etc.) from those organisms, tissues, cells or components thereof that display the “normal” (expected) respective characteristic. Characteristics which are normal or expected for one cell or tissue type, might be abnormal for a different cell or tissue type.

A “helper virus” for AAV refers to a virus allowing AAV (e.g. wild-type AAV) to be replicated and packaged by a mammalian cell. A variety of such helper viruses for AAV are known in the art, including adenoviruses, herpesviruses and poxviruses such as vaccinia. The adenoviruses encompass a number of different subgroups, although Adenovirus type 5 of subgroup C is most commonly used as a helper virus. Numerous adenoviruses of human, non-human mammalian and avian origin are known and available from depositories such as the ATCC. Viruses of the herpes family include, for example, herpes simplex viruses (HSV) and Epstein-Barr viruses (EBV), as well as cytomegaloviruses (CMV) and pseudorabies viruses (PRV); which are also available from depositories such as ATCC.

“Helper virus function(s)” refers to function(s) encoded in a helper virus genome allowing AAV replication and packaging (in conjunction with other requirements for replication and packaging described herein). As described herein, “helper virus function” may be provided in a number of ways, including by providing helper virus or providing, for example, polynucleotide sequences encoding the requisite function(s) to a producer cell in trans.

An “infectious” virion, virus or viral particle is one comprising a polynucleotide component deliverable into a cell tropic for the viral species. The term does not necessarily imply any replication capacity of the virus. As used herein, an “infectious” virus or viral particle is one that upon accessing a target cell, can infect a target cell, and can express a heterologous nucleic acid in a target cell. Thus, “infectivity” refers to the ability of a viral particle to access a target cell, enter a target cell, and express a heterologous nucleic acid in a target cell. Infectivity can refer to in vitro infectivity or in vivo infectivity. Assays for counting infectious viral particles are described elsewhere in this disclosure and in the art. Viral infectivity can be expressed as the ratio of infectious viral particles to total viral particles. Total viral particles can be expressed as the number of viral genome copies. The ability of a viral particle to express a heterologous nucleic acid in a cell can be referred to as “transduction.” The ability of a viral particle to express a heterologous nucleic acid in a cell can be assayed using a number of techniques, including assessment of a marker gene, such as a green fluorescent protein (GFP) assay (e.g., where the virus comprises a nucleotide sequence encoding GFP), where GFP is produced in a cell infected with the viral particle and is detected and/or measured; or the measurement of a produced protein, for example by an enzyme-linked immunosorbent assay (ELISA) or fluorescence-activated cell sorting (FACS).

A “replication-competent” virion or virus (e.g. a replication-competent AAV) refers to an infectious phenotypically wild-type virus, and is replicable in an infected cell (i.e. in the presence of a helper virus or helper virus functions). In the case of AAV, replication competence generally requires the presence of functional AAV packaging genes. In some embodiments, AAV vectors, as described herein, lack of one or more AAV packaging genes and are replication-incompetent in mammalian cells (especially in human cells). In some embodiments, AAV vectors lack any AAV packaging gene sequences, minimizing the possibility of generating replication competent AAV by recombination between AAV packaging genes and an incoming AAV vector. In many embodiments, AAV vector preparations as described herein are those containing few if any replication competent AAV (rcAAV, also referred to as RCA) (e.g., less than about 1 rcAAV per 10² AAV particles, less than about 1 rcAAV per 10⁴ AAV particles, less than about 1 rcAAV per 10⁸ AAV particles, less than about 1 rcAAV per 10¹² AAV particles, or no rcAAV).

“Recombinant,” e.g., as applied to a polynucleotide means, a product of various combinations of cloning, restriction or ligation steps, and other procedures resulting in a molecule distinct and/or different from one found in nature. For example, a recombinant virus can be a viral particle encapsidating a recombinant polynucleotide.

A “control element” or “control sequence” is a nucleotide sequence involved in an interaction of molecules contributing to the functional regulation of a polynucleotide, including replication, duplication, transcription, splicing, translation, or degradation of the polynucleotide. The regulation may affect the frequency, speed, or specificity of the process, and may be enhancing or inhibitory in nature. Control elements known in the art include, for example, transcriptional regulatory sequences such as promoters and enhancers. A promoter is a DNA region capable under certain conditions of binding RNA polymerase and initiating transcription usually downstream (in the 3′ direction) from the promoter.

“Operatively linked” or “operably linked” refers to a juxtaposition of genetic elements, wherein the elements are in a relationship permitting them to operate in the expected manner. For instance, a promoter is operatively linked to a sequence of interest (the sequence of interest can also be said to be operatively linked to the promoter) if the promoter helps initiate transcription of the sequence of interest. There may be intervening residues between the promoter and sequence of interest so long as this functional relationship is maintained.

“Heterologous” means derived from a genotypically distinct entity from the rest of the entity to it is being compared too. For example, a polynucleotide introduced by genetic engineering techniques into a plasmid or vector derived from a different species is a heterologous polynucleotide. A promoter removed from its native coding sequence and operatively linked to a different coding sequence is heterologous to that sequence. For example, an AAV including a heterologous nucleic acid encoding a heterologous gene product is an AAV including a nucleic acid not normally included in a naturally-occurring, wild-type AAV, and the encoded heterologous gene product is a gene product not normally encoded by a naturally-occurring, wild-type AAV. An AAV including a nucleic acid encoding a variant AAV capsid polypeptide includes a heterologous nucleic acid sequence. An AAV including a variant AAV capsid polypeptide includes a heterologous AAV capsid, and the capsid can be said to be heterologous to the nucleic acid that is encapsidated. Once transferred/delivered into a host cell, a heterologous polynucleotide, contained within the virion, can be expressed (e.g., transcribed, and translated if appropriate). Alternatively, a transferred/delivered heterologous polynucleotide into a host cell, contained within the virion, need not be expressed.

The terms “polypeptide,” “peptide,” and “protein” are used interchangeably herein to refer to polymers of amino acids of any length. The “polypeptides,” “proteins” and “peptides” encoded by the “polynucleotide sequences,” include full-length native sequences, as with naturally occurring proteins, as well as functional subsequences, modified forms or sequence variants so long as the subsequence, modified form or variant retains some degree of the intended functionality. The terms also encompass a modified amino acid polymer; for example, disulfide bond formation, glycosylation, lipidation, phosphorylation, methylation, carboxylation, deamidation, acetylation, or conjugation with a labeling component. Polypeptides such as anti-angiogenic polypeptides, neuroprotective polypeptides, and the like, when discussed in the context of delivering a gene product to a mammalian subject, and compositions therefor, refer to the respective intact polypeptide, or any fragment or genetically engineered derivative thereof, retaining the desired biochemical function of the intact protein.

“Isolated” means altered or removed from the natural state. For example, a nucleic acid or a polypeptide naturally present in a living animal is not “isolated,” but the same nucleic acid or polypeptide partially or completely separated from the coexisting materials of its natural state is “isolated.” An isolated nucleic acid or protein can exist in substantially purified form, or can exist in a non-native environment such as, for example, a host cell. An “isolated” plasmid, nucleic acid, vector, virus, virion, host cell, or other substance refers to a preparation of the substance devoid of at least some of the other components present where the substance or a similar substance naturally occurs or from which it is initially prepared. Thus, for example, an isolated substance may be prepared by using a purification technique to enrich it from a source mixture. Enrichment can be measured on an absolute basis, such as weight per volume of solution, or it can be measured in relation to a second, potentially interfering substance present in the source mixture. Increasing enrichments of the embodiments of this invention are increasingly more isolated. An isolated plasmid, nucleic acid, vector, virus, host cell, or other substance is in some embodiments purified, e.g., from about 80% to about 90% pure, at least about 90% pure, at least about 95% pure, at least about 98% pure, or at least about 99%, or more, pure.

By the term “highly conserved” is meant at least about 80% identity, preferably at least 90% identity, and more preferably, over about 97% identity. Identity is readily determined by one of skill in the art by resort to algorithms and computer programs known by those of skill in the art.

As used herein, the term “wild-type” refers to a gene or gene product isolated from a naturally occurring source or having a naturally occurring sequence (e.g., a wild type protein with a naturally occurring amino acid sequence can be isolated from a natural source or from a synthetic source, but would still be considered a wild type protein). In contrast, the term “modified,” “variant,” or “mutant” refers to a gene or gene product that possesses modifications in sequence and/or functional properties (i.e., altered characteristics) when compared to the wild-type gene or gene product.

As used herein, the terms “treatment,” “treating,” and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment,” as used herein, covers any treatment of a disease in a mammal, particularly in a human, and includes: (a) preventing the disease from occurring in a subject predisposed to the disease or at risk of acquiring the disease but has not yet been diagnosed as having it; (b) inhibiting the disease, i.e., arresting its development; and (c) relieving the disease, i.e., causing regression of the disease.

As used herein, the terms “therapy” or “therapeutic regimen” refer to those activities taken to prevent, treat or alter a disease or disorder, e.g., a course of treatment intended to reduce or eliminate at least one sign or symptom of a disease or disorder using pharmacological, surgical, dietary and/or other techniques. A therapeutic regimen may include a prescribed dosage of one or more compounds and/or or surgery. Therapies will most often be beneficial and reduce or eliminate at least one sign or symptom of the disorder or disease state, but in some instances the effect of a therapy will have non-desirable or side-effects. The effect of therapy will also be impacted by the physiological state of the subject, e.g., age, gender, genetics, weight, other disease conditions, etc.

The term “therapeutically effective amount” refers to the amount of the subject compound or composition that will elicit the intended biological, physiologic, clinical or medical response of a cell, tissue, organ, system, or subject that is being sought by the researcher, veterinarian, medical doctor or other clinician. The term “therapeutically effective amount” includes that amount of a compound or composition that, when administered, is sufficient to treat one or more of the signs or symptoms of the disorder or disease being treated (e.g., chronic pain). The therapeutically effective amount will vary depending on the compound or composition, the disease and its severity and the age, weight, etc., of the subject to be treated. A “therapeutically effective amount” will fall in a relatively broad range determinable through experimentation and/or clinical trials. For example, for in vivo injection, a therapeutically effective dose can in some cases be on the order of from about 10⁶ to about 10¹⁵ of AAV virions per kilogram bodyweight of the subject. In some embodiments, a therapeutically effective dose will be on the order of from about 10⁸ to 10¹² AAV virions per kilogram bodyweight of the subject. Other effective dosages can be readily established by one of ordinary skill in the art through routine trials establishing dose response curves.

The terms “individual,” “subject,” and “patient” are used interchangeably herein, and refer to a mammal, including, but not limited to, human and non-human primates, including simians and humans; mammalian sport animals (e.g., horses); mammalian farm animals (e.g., sheep, goats, etc.); mammalian pets (dogs, cats, etc.); and rodents (e.g., mice, rats, etc.) for whom diagnosis, treatment, or therapy is desired, particularly humans.

The terms “pharmaceutically acceptable” and “physiologically acceptable” mean a biologically acceptable formulation, gaseous, liquid or solid, or mixture thereof, suitable for one or more routes of administration, in vivo delivery or contact. A “pharmaceutically acceptable” or “physiologically acceptable” composition is a material that is not biologically or otherwise undesirable, e.g., the material may be administered to a subject without causing substantial undesirable biological effects. Thus, such a pharmaceutical composition may be used, for example in administering an AAV vector or AAV virion as disclosed herein, or transformed cell to a subject.

The phrase a “unit dosage form” as used herein refers to physically discrete units suited as unitary dosages for the subject to be treated; each unit containing a predetermined quantity optionally in association with a pharmaceutical carrier (excipient, diluent, vehicle or filling agent) which, when administered in one or more doses, produces a desired effect (e.g., prophylactic or therapeutic effect). In some embodiments, unit dosage forms may be within, for example, ampules and vials, including a liquid composition, or a composition in a freeze-dried or lyophilized state; a sterile liquid carrier, for example, can be added prior to administration or delivery in vivo. Individual unit dosage forms can be included in multi-dose kits or containers. AAV vectors or AAV virions, and pharmaceutical compositions thereof can be packaged in single or multiple unit dosage form for ease of administration and uniformity of dosage.

An “effective amount” or “sufficient amount” refers to an amount providing, in single or multiple doses, alone or in combination, with one or more other compositions (therapeutic agents such as a drug), treatments, protocols, or therapeutic regimens agents (including, for example, vaccine regimens), a detectable response of any duration of time (long or short term), an expected or desired outcome in or a benefit to a subject of any measurable or detectable degree or for any duration of time (e.g., for minutes, hours, days, months, years, or cured).

The doses of an “effective amount” or “sufficient amount” for treatment (e.g., to ameliorate or to provide a therapeutic benefit or improvement) typically are effective to provide a response to one, multiple or all adverse symptoms, consequences or complications of the disease or disorder (e.g., chronic pain), one or more adverse symptoms, disorders, illnesses, pathologies, or complications, for example, caused by or associated with the disease, to a measurable extent, although decreasing, reducing, inhibiting, suppressing, limiting or controlling progression or worsening of the disease is also a satisfactory outcome.

“Prophylaxis” and grammatical variations thereof mean a method in which contact, administration or in vivo delivery to a subject is prior to disease. Administration or in vivo delivery to a subject can be performed prior to development of an adverse symptom, condition, complication, etc. caused by or associated with the disease. For example, a screen (e.g., genetic) can be used to identify such subjects as candidates for the described methods and uses, but the subject may not manifest the disease. Such subjects therefore include those screened positive for an insufficient amount or a deficiency in a functional gene product (protein), or producing an aberrant, partially functional or non-functional gene product (protein), leading to disease; and subjects screening positive for an aberrant, or defective (mutant) gene product (protein) leading to disease, even though such subjects do not manifest symptoms of the disease.

The phrases “tropism” and “transduction” are interrelated, but there are differences. The term “tropism” as used herein refers to the ability of an AAV vector or virion to infect one or more specified cell types, but can also encompass how the vector functions to transduce the cell in the one or more specified cell types; i.e., tropism refers to preferential entry of the AAV vector or virion into certain cell or tissue type(s) and/or preferential interaction with the cell surface that facilitates entry into certain cell or tissue types, optionally and preferably followed by expression (e.g., transcription and, optionally, translation) of sequences carried by the AAV vector or virion in the cell, e.g., for a recombinant virus, expression of the heterologous nucleotide sequence(s). As used herein, the term “transduction” refers to the ability of an AAV vector or virion to infect one or more particular cell types; i.e., transduction refers to entry of the AAV vector or virion into the cell and the transfer of genetic material contained within the AAV vector or virion into the cell to obtain expression from the vector genome. In some cases, but not all cases, transduction and tropism may correlate.

An “oligonucleotide” or “polynucleotide” is a nucleic acid ranging from at least 2, preferably at least 8, 15 or 25 nucleotides in length, but may be up to 50, 100, 1000, or 5000 nucleotides long or a compound that specifically hybridizes to a polynucleotide. Polynucleotides include sequences of deoxyribonucleic acid (DNA) or ribonucleic acid (RNA) or mimetics thereof which may be isolated from natural sources, recombinantly produced or artificially synthesized. A further example of a polynucleotide of the present invention may be a peptide nucleic acid (PNA). (See U.S. Pat. No. 6,156,501 which is hereby incorporated by reference in its entirety.) The invention also encompasses situations in which there is a nontraditional base pairing such as Hoogsteen base pairing which has been identified in certain tRNA molecules and postulated to exist in a triple helix. “Polynucleotide” and “oligonucleotide” are used interchangeably in this disclosure. It will be understood that when a nucleotide sequence is represented herein by a DNA sequence (e.g., A, T, G, and C), this also includes the corresponding RNA sequence (e.g., A, U, G, C) in which “U” replaces “T”.

As used herein, “polynucleotide” includes cDNA, RNA, DNA/RNA hybrid, antisense RNA, ribozyme, genomic DNA, synthetic forms, and mixed polymers, both sense and antisense strands, and may be chemically or biochemically modified to exhibit non-natural or derivatized, synthetic, or semi-synthetic nucleotide bases. Also, contemplated are alterations of a wild type or synthetic gene, including but not limited to deletion, insertion, substitution of one or more nucleotides, or fusion to other polynucleotide sequences.

“Sample” or “biological sample” as used herein means a biological material isolated from a subject. The biological sample may contain any biological material suitable for detecting a mRNA, polypeptide or other marker of a physiologic or pathologic process in a subject, and may comprise fluid, tissue, cellular and/or non-cellular material obtained from the individual.

The term “antibody,” as used herein, refers to an immunoglobulin molecule which is able to specifically bind to a specific epitope on an antigen. Antibodies can be intact immunoglobulins derived from natural sources or from recombinant sources and can be immunoreactive portions of intact immunoglobulins. Antibodies may exist in a variety of forms including, for example, polyclonal antibodies, monoclonal antibodies, intracellular antibodies (“intrabodies”), Fv, Fab and F(ab)2, as well as single chain antibodies (scFv), heavy chain antibodies, such as camelid antibodies, synthetic antibodies, chimeric antibodies, and a humanized antibodies (Harlow et al., 1999, Using Antibodies: A Laboratory Manual, Cold Spring Harbor Laboratory Press, NY; Harlow et al., 1989, Antibodies: A Laboratory Manual, Cold Spring Harbor, New York; Houston et al., 1988, Proc. Natl. Acad. Sci. USA 85:5879-5883; Bird et al., 1988, Science 242:423-426).

An “antibody heavy chain,” as used herein, refers to the larger of the two types of polypeptide chains present in all antibody molecules in their naturally occurring conformations.

An “antibody light chain,” as used herein, refers to the smaller of the two types of polypeptide chains present in all antibody molecules in their naturally occurring conformations. □ and □ light chains refer to the two major antibody light chain isotypes.

By the term “synthetic antibody” as used herein, is meant an antibody which is generated using recombinant DNA technology, such as, for example, an antibody expressed by a bacteriophage as described herein. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art.

As used herein, an “immunoassay” refers to any binding assay that uses an antibody capable of binding specifically to a target molecule to detect and quantify the target molecule.

By the term “specifically binds,” as used herein, e.g., with respect to a protein specifically binding another molecule such as another protein, it is meant preferential binding over other possible binding partners. For example an antibody that specifically binds antigen X will preferentially bind to antigen X over other antigens. Another non-limiting example of specific binding is a receptor binding to its ligand. Similarly, the term “targets”, e.g., in the context of antibodies that “target” a particular antigen, is used to refer to the specific binding partner of a given molecule. For example, an agent (e.g., an antibody) that “targets” a particular protein/antigen specifically binds that protein/antigen in the sense that it preferentially binds to that particular protein/antigen over other proteins/antigens. Likewise, an RNA agent such as an shRNA “targets” a particular RNA target (due to hybridization) because it has complementarity to and preferentially hybridizes to a target sequence of the target RNA over sequences of other RNAs.

The term “coding sequence,” as used herein, means a sequence of a nucleic acid or its complement, or a part thereof, that can be transcribed and/or translated to produce the mRNA and/or the polypeptide or a fragment thereof. Coding sequences include exons in a genomic DNA or immature primary RNA transcripts, which are joined together by the cell's biochemical machinery to provide a mature mRNA. The anti-sense strand is the complement of such a nucleic acid, and the coding sequence can be deduced therefrom. In contrast, the term “non-coding sequence,” as used herein, means a sequence of a nucleic acid or its complement, or a part thereof, that is not translated into amino acid in vivo, or where tRNA does not interact to place or attempt to place an amino acid. Non-coding sequences include both intron sequences in genomic DNA or immature primary RNA transcripts, and gene-associated sequences such as promoters, enhancers, silencers, and the like.

As used herein, the terms “complementary” or “complementarity” are used in reference to polynucleotides (i.e., a sequence of nucleotides) related by the base-pairing rules. For example, the sequence “A-G-T,” is complementary to the sequence “T-C-A.” Complementarity may be “partial,” in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands. This is of particular importance in amplification reactions, as well as detection methods that depend upon binding between nucleic acids.

A “disease” is a state of health of an animal wherein the animal cannot maintain homeostasis, and wherein if the disease is not ameliorated then the animal's health continues to deteriorate. In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., guide RNA, rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

“Homologous”, “identical,” or “identity” as used herein in the context of two or more nucleic acids or polypeptide sequences means that the sequences have a specified percentage of residues that are the same over a specified region. The percentage can be calculated by optimally aligning the two sequences, comparing the two sequences over the specified region, determining the number of positions at which the identical residue occurs in both sequences to yield the number of matched positions, dividing the number of matched positions by the total number of positions in the specified region, and multiplying the result by 100 to yield the percentage of sequence identity. In cases where the two sequences are of different lengths or the alignment produces one or more staggered ends and the specified region of comparison includes only a single sequence, the residues of the single sequence are included in the denominator but not the numerator of the calculation. When comparing DNA and RNA, thymine (T) and uracil (U) can be considered equivalent. Identity can be performed manually or by using a computer sequence algorithm such as BLAST or BLAST 2.0.

“Instructional material,” as that term is used herein, includes a publication, a recording, a diagram, or any other medium of expression which can be used to communicate the usefulness of the nucleic acid, peptide, polypeptide, and/or compound of the invention in the kit for identifying or alleviating or treating the various diseases or disorders recited herein. Optionally, or alternately, the instructional material may describe one or more methods of identifying or alleviating the diseases or disorders in a cell or a tissue of a subject. The instructional material of the kit may, for example, be affixed to a container that contains the nucleic acid, polypeptide, and/or compound of the invention or be shipped together with a container that contains the nucleic acid, polypeptide, and/or compound. Alternatively, the instructional material may be shipped separately from the container with the intention that the recipient uses the instructional material and the compound cooperatively.

As used herein, the terms “purify” and “purified” in the context of a protein refers to level of purity that allows for the effective use of the protein, e.g., in vitro, ex vivo, or in vivo. For a protein to be useful for a given application, it should be substantially free of contaminants, other proteins, and/or chemicals that could interfere with the use of that protein in such application, or that at least would be undesirable for inclusion with the protein of interest. Such applications include that preparation of therapeutic compositions, the administration of the protein in a therapeutic composition, and other methods disclosed herein. Preferably, a “purified” protein, as referenced herein, is a protein that can be produced by any method (i.e., by direct purification from a natural source, recombinantly, or synthetically), and that has been purified from other protein components such that the protein comprises at least about 75% weight/weight of the total protein in a given composition, 80% weight/weight of the total protein in a given composition, and more preferably, at least about 85%, and more preferably at least about 90%, and more preferably at least about 91%, and more preferably at least about 92%, and more preferably at least about 93%, and more preferably at least about 94%, and more preferably at least about 95%, and more preferably at least about 96%, and more preferably at least about 97%, and more preferably at least about 98%, and more preferably at least about 99% weight/weight of the total protein in a given composition. As an example, a purified polypeptide is a polypeptide which has been separated from other components with which it might normally be associated in its naturally occurring state (e.g., if the protein is a naturally existing protein) and from components with which it may be associated while inside of a cell or in extracellular milieu. For example, in some cases a protein can be purified from a cellular lysate (e.g., from a lysate of bacterial cells in which the protein was exogenously expressed). As another example a protein can be purified from an extracellular medium, e.g., from culture medium into which cells (e.g., yeast cells) have secreted the protein.

By the term “modulating,” as used herein, is meant mediating a detectable increase or decrease in the activity and/or level of a mRNA, polypeptide, or a response in a subject compared with the activity and/or level of the mRNA, polypeptide or response in the subject in the absence of a treatment or compound, and/or compared with the activity and/or level of the mRNA, polypeptide, or response in an otherwise identical but untreated subject. The term encompasses activating, inhibiting and/or otherwise affecting a native signal or response thereby mediating a beneficial therapeutic, prophylactic, or other desired response in a subject, for example, a human.

A “nucleic acid” refers to a polynucleotide and includes poly-ribonucleotides and poly-deoxyribonucleotides. Nucleic acids according to the present invention may include any polymer or oligomer of pyrimidine and purine bases, preferably cytosine, thymine, and uracil, and adenine and guanine, respectively. (See Albert L. Lehninger, Principles of Biochemistry, at 793-800 (Worth Pub. 1982) which is herein incorporated in its entirety for all purposes). Indeed, the present invention contemplates any deoxyribonucleotide, ribonucleotide or peptide nucleic acid component, and any chemical variants thereof, such as methylated, hydroxymethylated or glucosylated forms of these bases, and the like. The polymers or oligomers may be heterogeneous or homogeneous in composition, and may be isolated from naturally occurring sources or may be artificially or synthetically produced. In addition, the nucleic acids may be DNA or RNA, or a mixture thereof, and may exist permanently or transitionally in single-stranded or double-stranded form, including homoduplex, heteroduplex, and hybrid states.

Ranges: throughout this disclosure, various aspects can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 2, 1 to 3, 1 to 4, 1 to 5, 2 to 3, 2 to 4, 2 to 5, 2 to 6, etc., as well as individual numbers within that range, for example, 1, 2, 2.7, 3, 4, 5, 5.3, and 6. This applies regardless of the breadth of the range.

### Kits

The present disclosure provides kits/systems for carrying out a subject method. Such kits comprise various combinations of components useful in any of the methods described elsewhere herein. In some embodiments a subject kit includes a subject retrograde-enhanced clade E variant AAV (virion) (e.g. AAV8-retro or rhi0-retro), a retrograde-enhanced clade E variant AAV vector, a nucleic acid encoding a subject retrograde-enhanced clade E variant AAV capsid protein, a subject transduction system (as described in detail elsewhere herein), a nucleic acid encoding an RNAi agent such as an shRNA that targets CamKv, a nucleic acid encoding an CRISPR/Cas guide RNA that targets CamKv, or any combination thereof. In some cases, a subject kit can further include a cell or population of cells for packaging/generating a subject retrograde-enhanced clade E variant AAV.

In a further embodiment, the kit comprises the components of an assay for monitoring the effectiveness of a treatment administered to a subject in need thereof, containing instructional material and the components for determining whether the level of IL-18 signaling in a biological sample obtained from the subject is modulated during or after administration of the treatment.

A kit can further include one or more additional reagents, where such additional reagents can be any convenient reagent. Components of a subject kit can be in separate containers; or can be combined in a single container. In some cases one or more of a kit's components are pharmaceutically formulated for administration to a human.

In addition to above-mentioned components, a subject kit can further include instructions for using the components of the kit to practice the subject methods (e.g., dosing instructions, instructions to administer the component(s) to an individual with an ongene-negative cancer such as a lung cancer (e.g., lung adenocarcinoma). The instructions for practicing the subject methods are generally recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or subpackaging) etc. In some embodiments, the instructions are present as an electronic storage data file present on a suitable computer readable storage medium, e.g. CD-ROM, diskette, flash drive, etc. In some embodiments, the actual instructions are not present in the kit, but means for obtaining the instructions from a remote source, e.g. via the internet, are provided. An example of this embodiment is a kit that includes a web address where the instructions can be viewed and/or from which the instructions can be downloaded. As with the instructions, this means for obtaining the instructions is recorded on a suitable substrate.

### Exemplary Non-Limiting Aspects of the Disclosure

Aspects, including embodiments, of the present subject matter described above may be beneficial alone or in combination, with one or more other aspects or embodiments. Without limiting the foregoing description, certain non-limiting aspects of the disclosure are provided below. As will be apparent to those of ordinary skill in the art upon reading this disclosure, each of the individually numbered aspects may be used or combined with any of the preceding or following individually numbered aspects. This is intended to provide support for all such combinations of aspects and is not limited to combinations of aspects explicitly provided below. It will be apparent to one of ordinary skill in the art that various changes and modifications can be made without departing from the spirit or scope of the invention.

1. A retrograde-enhanced clade E variant adeno-associated virus (AAV) capsid protein, comprising the amino acid sequence LADQDYTKTA (SEQ ID NO: 30) inserted into a clade E AAV capsid protein.

2. The retrograde-enhanced clade E variant AAV capsid protein of 1, wherein the LADQDYTKTA (SEQ ID NO: 30) sequence immediately follows a QQQN (SEQ ID NO: 28), QQTN (SEQ ID NO: 29), QQQD (SEQ ID NO: 30), or QQAN (SEQ ID NO: 31) sequence.

3. The retrograde-enhanced clade E variant AAV capsid protein of 1 or 2, comprising an amino acid sequence having 85% or more sequence identity with the amino acid sequence set forth in any one of SEQ ID NOs: 32-57.

4. The retrograde-enhanced clade E variant AAV capsid protein of 1 or 2, comprising an amino acid sequence having 85% or more sequence identity with the AAV8-retro capsid amino acid sequence set forth in SEQ ID NO: 32 or with the rh10-retro capsid amino acid sequence set for in SEQ ID NO: 57.

5. The retrograde-enhanced clade E variant AAV capsid protein of 1, comprising the amino acid sequence LADQDYTKTA (SEQ ID NO: 30) inserted into an AAV8 capsid protein or into an rh10 capsid protein.

6. The retrograde-enhanced clade E variant AAV capsid protein of 1, where the retrograde-enhanced clade E variant AAV capsid protein is an AAV8-retro capsid protein comprising the amino acid sequence set forth in SEQ ID NO 32 or an rh10-retro capsid protein comprising the amino acid sequence set forth in SEQ ID NO 57.

7. A transduction system comprising one or more nucleic acids, where said one or more nucleic acids comprises a nucleotide sequence that encodes the retrograde-enhanced clade E variant AAV capsid protein of any one of 1-6.

8. The transduction system of 7, where said one or more nucleic acids further comprises a transgene sequence flanked by inverted terminal repeat sequences (ITRs).

9. The transduction system of 8, wherein the transgene sequence encodes a non-coding RNA.

10. The transduction system of 9, wherein the non-coding RNA is a short hairpin RNA (shRNA).

11. The transduction system of 10, wherein the shRNA targets CamKv.

12. The transduction system of 9, wherein the non-coding RNA is a CRISPR/Cas guide RNA.

13. The transduction system of 8, wherein the transgene sequence encodes a polypeptide.

14. The transduction system of 13, wherein the polypeptide is a genome-targeting protein.

15. A retrograde-enhanced recombinant AAV particle comprising:
(a) the retrograde-enhanced clade E variant AAV capsid protein of any one of 1-6; and
(b) a nucleic acid comprising a transgene sequence.

16. The retrograde-enhanced recombinant AAV particle of 15, wherein the transgene sequence encodes a non-coding RNA.

17. The retrograde-enhanced recombinant AAV particle of 16, wherein the non-coding RNA is a short hairpin RNA (shRNA).

18. The retrograde-enhanced recombinant AAV particle of 17, wherein the shRNA targets CamKv.

19. The retrograde-enhanced recombinant AAV particle of 17, wherein the non-coding RNA is a CRISPR/Cas guide RNA.

20. The retrograde-enhanced recombinant AAV particle of 16, wherein the transgene sequence encodes a polypeptide.

21. The retrograde-enhanced recombinant AAV particle of 20, wherein the polypeptide is a genome-targeting protein.

22. The retrograde-enhanced recombinant AAV particle of 21, wherein the genome-targeting protein is a CRISPR/Cas effector protein, a zinc finger fusion, or a TALE fusion.

23. A method of making a retrograde-enhanced recombinant AAV particle, the method comprising: introducing the transduction system of any one of 7-14 into a eukaryotic cell, wherein the eukaryotic cell produces said retrograde-enhanced recombinant AAV particle.

24. A method of expressing a transgene of interest in a neuron, the method comprising: contacting the neuron with the retrograde-enhanced recombinant AAV particle of any one of 15-22.

25. The method of 24, wherein said contacting occurs in an individual's spinal cord or thalamus.

26. The method of 24 or 25, wherein the neuron is a spinal cord projecting neuron or a corticothalamic projecting neuron.

27. The method of 26, wherein the spinal cord-projecting neuron is a neuron of the rostral ventromedial medulla (RVM).

28. The method of 26, wherein the spinal cord-projecting neuron is a neuron of the locus coeruleus (LC).

29. The method of any one of 24-28, wherein the neuron is an opioid receptor mu 1 (OPRM1) expressing neuron.

30. A method of treating an individual in need, the method comprising:
administering to an individual who has chronic pain a therapy that reduces CamKv activity in opioid receptor mu 1 (OPRM1) expressing neurons of the individual's rostral ventromedial medulla (RVM).

31. The method of 30, wherein said therapy is an agent that reduces expression of CamKv in said neurons.

32. The method of 31, wherein said agent comprises a retrograde-enhanced recombinant AAV particle that comprises a retrograde-enhanced clade E variant adeno-associated virus (AAV) capsid protein comprising the amino acid sequence LADQDYTKTA (SEQ ID NO: 30) inserted into a clade E AAV capsid protein.

33. The method of 32, wherein the LADQDYTKTA (SEQ ID NO: 30) sequence immediately follows a QQQN (SEQ ID NO: 28), QQTN (SEQ ID NO: 29), QQQD (SEQ ID NO: 30), or QQAN (SEQ ID NO: 31) sequence.

34. The method of 32 or 33, wherein the retrograde-enhanced clade E variant AAV capsid protein comprises an amino acid sequence having 85% or more sequence identity with the amino acid sequence set forth in any one of SEQ ID NOs: 32-57.

35. The method of 32 or 33, wherein the retrograde-enhanced clade E variant AAV capsid protein comprises an amino acid sequence having 85% or more sequence identity with the AAV8-retro capsid amino acid sequence set forth in SEQ ID NO: 32 or with the rh10-retro capsid amino acid sequence set for in SEQ ID NO: 57.

36. The method of 32 or 33, wherein the retrograde-enhanced clade E variant AAV capsid protein comprises the amino acid sequence LADQDYTKTA (SEQ ID NO: 30) inserted into an AAV8 capsid protein or into an rh10 capsid protein.

37. The method of 32, wherein the retrograde-enhanced clade E variant AAV capsid protein is an AAV8-retro capsid protein comprising the amino acid sequence set forth in SEQ ID NO 32 or an rh10-retro capsid protein comprising the amino acid sequence set forth in SEQ ID NO 57.

38. The method of any one of 32-37, wherein the retrograde-enhanced recombinant AAV particle comprises a nucleic acid comprising a transgene sequence.

39. The method of 38, wherein the transgene sequence encodes an shRNA that targets CamKv mRNA.

40. The method of 38, wherein the transgene sequence encodes a CRISPR/Cas guide RNA that targets CamKv.

41. The method of 38, wherein the transgene sequence encodes a polypeptide.

42. The method of 41, wherein the polypeptide is a genome-targeting protein.

43. The method of 42, wherein the genome-targeting protein is a CRISPR/Cas effector protein or fusion protein thereof, a zinc finger fusion, or a TALE fusion.

44. The method of 31, wherein said agent comprises an RNAi agent that targets CamKv mRNA.

45. The method of 44, wherein said RNAi agent is an shRNA or a DNA encoding the shRNA.

46. The method of 31, wherein said agent comprises a protein or a nucleic acid encoding the protein, wherein the protein is a CRISPR/Cas effector protein or fusion protein thereof, a zinc finger fusion, or a TALE fusion.

47. The method of 46, wherein said agent comprises: (i) the CRISPR/Cas effector protein or fusion protein thereof, or a nucleic acid encoding the CRISPR/Cas effector protein or fusion protein thereof; and (ii) a CRISPR/Cas guide RNA that targets CamKv.

48. The method of any one of 31-47, comprising injecting said agent into the individual's spinal cord.

49. The method of 30, wherein said therapy comprises reducing excitatory input into the RVM from RVM projecting lateral superior colliculus (lSCIndG) neurons.

50. The method of 30, wherein said therapy comprises increasing inhibitory input into the RVM.

51. The method of 50, wherein said inhibitory input into the RVM is from zona incerta neurons.

52. The method of 51, wherein the therapy comprises deep brain stimulation of said zona incerta neurons.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather should be construed to encompass any and all variations which become evident as a result of the teaching provided herein.

Without further description, it is believed that one of ordinary skill in the art can, using the preceding description and the following illustrative examples, make and utilize the present invention and practice the claimed methods. The following working examples therefore are not to be construed as limiting in any way the remainder of the disclosure.

### Example 1: Collicular Input Upregulating Pseudokinase CaMKv in Medulla Drives Mechanical Pain

Persistent mechanical pain caused by inflammation or nerve injury is a debilitating clinical problem. The spinal cord-projecting neurons in the rostral ventromedial medulla (RVM^{SC} neurons) play active roles in pain facilitation¹⁻⁴. However, the underlying circuitry and molecular mechanisms remain largely unknown. The below experiments demonstrate that acute activation of OPRM1⁺ RVM^{SC} neurons does not facilitate pain in normal mice, but activity of these neurons is required for both initiation and maintenance of mechanical hypersensitivity in mouse models of inflammatory and neuropathic pain. The work described herein led to the surprising finding that the lateral superior colliculus rather than the traditionally assumed periaqueductal gray, provides the excitatory input onto the OPRM1⁺ RVM^{SC} neurons that drives mechanical hypersensitivity. Combining Ribotag RNA profiling and pathway manipulation, the work described herein established that collicular inputs are essential for upregulating pseudokinase CaMKv in the OPRM1⁺ RVM^{SC} neurons after nerve injury, and demonstrated that up- or down- regulation of CaMKv is sufficient to drive or reverse mechanical hypersensitivity. Together, the results reveal a collicular-medulla-spinal cord pathway that drives persistent pain and identify CaMKv as a key molecular determinant of mechanical hypersensitivity.

The periaqueductal gray (PAG)-rostral ventromedial medulla (RVM, including the raphe magnus and gigantocellular reticular nuclei) system have been established as important nodes in descending pain modulation network. Electrical stimulation or local injection of morphine into PAG or RVM elicits a potent analgesic effect. The PAG does not project directly to the spinal cord. Instead, it innervates the RVM, which contains descending neurons that interact with local pain circuitry in the spinal cord. Classic in vivo electrophysiological studies classified the spinal cord-projecting neurons in the RVM (RVM^{SC} neurons) into ON-, OFF- and Neutral cells based on whether they are activated, inhibited or unaffected by nociceptive stimuli. Moreover, morphine directly inhibits ON-cells and indirectly activates OFF-cells via disinhibition. These response properties lead to the hypothesis that ON-cells activity facilitates pain whereas OFF-cells activity inhibits pain. However, there are two technical challenges that prevented previous studies from selectively manipulating activity of descending ON- or OFF-cells in awake behaving mice, therefore this hypothesis has not been directly tested.

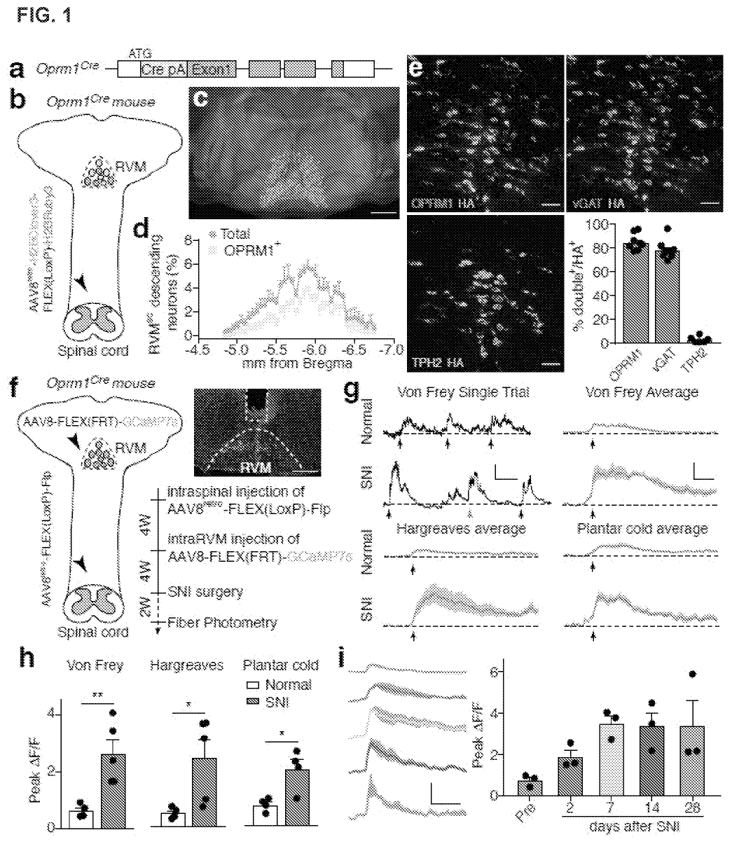

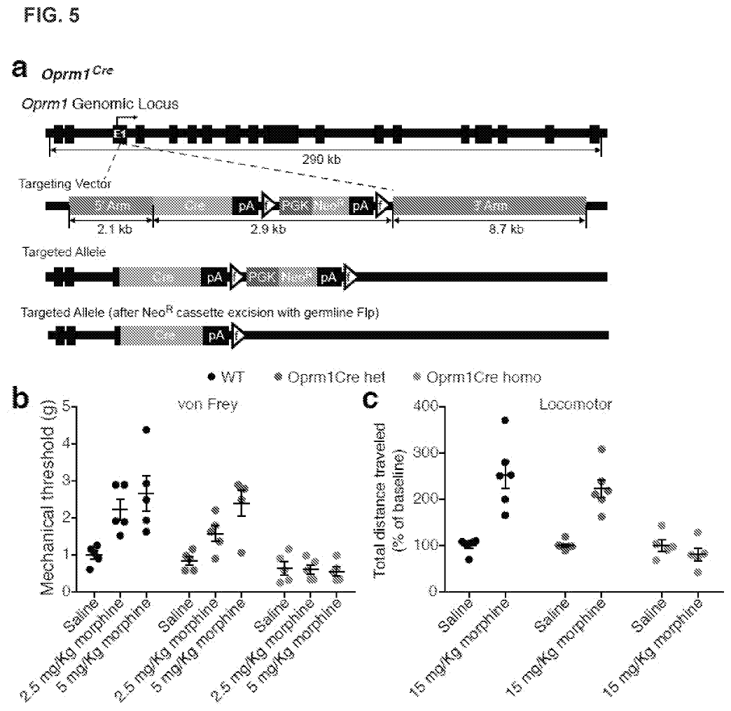

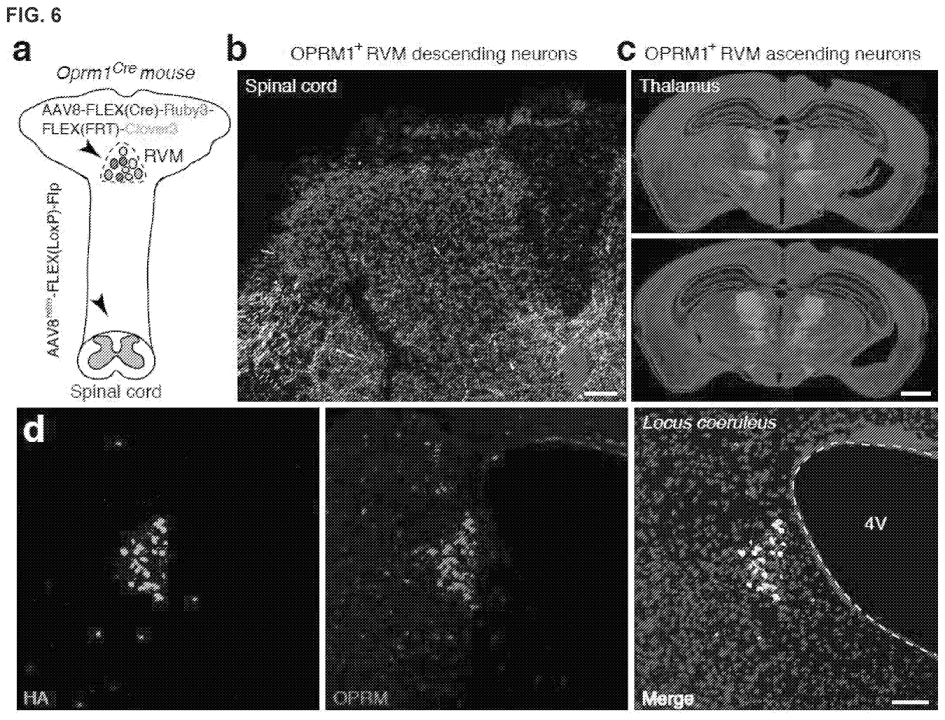

The first challenge is the lack of genetic access to different types of RVM neurons. Inhibition of ON-cells by intra RVM injection of morphine suggests the expression of p-opioid receptor (encoded by the Oprm1 gene) in ON-cells. A knockin mouse line was therefore generated to express Cre recombinase from the endogenous Oprm1 locus (Oprm1-Cre mice, FIG. 1a and FIG. 5). However, besides RVM^{SC} neurons, local interneurons or ascending neurons in the RVM could also expresses the μ-opioid receptor (FIG. 6a-c). Therefore, the second challenge is to gain specific and effective access to the RVM^{SC} neurons. To address this challenge, a neonatal spinal virus injection procedure was optimized and spreading of adeno-associated virus (AAV) broadly in the dorsal horn between cervical and lumbar spinal cord was achieved (FIG. 7a,b). A new retrograde AAV was then developed by inserting the decapeptide LADQDYTKTA between N590 and T591 of the AAV8 capsid (AAV8-retro) (FIG. 7c). This modification surprisingly led to about a 2.3 fold increase in retrogradely labeled RVM^{SC} neurons compared to AAV2-retro (FIG. 7d,e). AAV8-retro-H2B-Clover3-FLEX(LoxP)-H2B-Ruby3 was injected into the spinal cord of Oprm1-Cre mice at P1.5 to express nuclear-localized Ruby3 and Clover3 in Oprm1+ and all spinal projecting neurons, respectively (FIG. 1b). About 5600 RVM^{SC} neurons were labeled per mouse ranging from bregma −4.7 to −6.5 mm and 65% of which express the p-opioid receptor (FIGS. 1c and d). Using RNAscope in situ hybridization, it was found that the majority of Oprm1+ RVM^{SC} neurons are GABAergic but not serotonergic (FIG. 1e).

Besides RVM, many neurons in locus coeruleus (LC) were also labeled (FIG. 6d), which is consistent with robust Oprm1 expression in LC16. An intersectional strategy was therefore devised to specifically target Oprm1+ RVM^{SC} neurons but spare Oprm1+ LC→SC neurons. AAV8-retro expressing Cre-dependent Flp (AAV8-retro-FLEX(LoxP)-Flp, 1 μl) was first injected into the spinal cord of Oprm1-Cre mice at P1.5, to express Flp recombinase in all Oprm1+ spinal cord-projecting neurons. Six weeks later, AAV8 expressing Flp-dependent effectors (AAV8-FLEX(FRT)-effectors) was then injected into the RVM of these mice, to achieve restricted expression of a variety of effectors in Oprm1+ RVM^{SC} neurons. Using this intersectional strategy, Oprm1+ RVM^{SC} neurons were transduced with genetically encoded neuronal activity sensor jGCaMP7s18, and then fiber photometry was used to record in vivo responses of Oprm1+ RVM^{SC} neurons during Von Frey, Hargreaves and plantar cold tests (FIG. 1f)¹⁹. Oprm1+ RVM^{SC} neurons showed time-locked calcium transients when mice withdrew their paw to both temperature and mechanical stimuli. After a neuropathic pain state was induced by spared nerve injury (SNI), the Oprm1+ RVM^{SC} neuron responses were significantly increased (FIG. 1g-i)²⁰. These observations provided direct evidence supporting that Oprm1+ RVM^{SC} neurons are ON-cells. Interestingly, calcium events in SNI mice associated with spontaneous paw flinching was often observed (FIG. 1g, red arrow), consistent with the idea that activity of ON-cells reflects pain but not sensory stimuli.

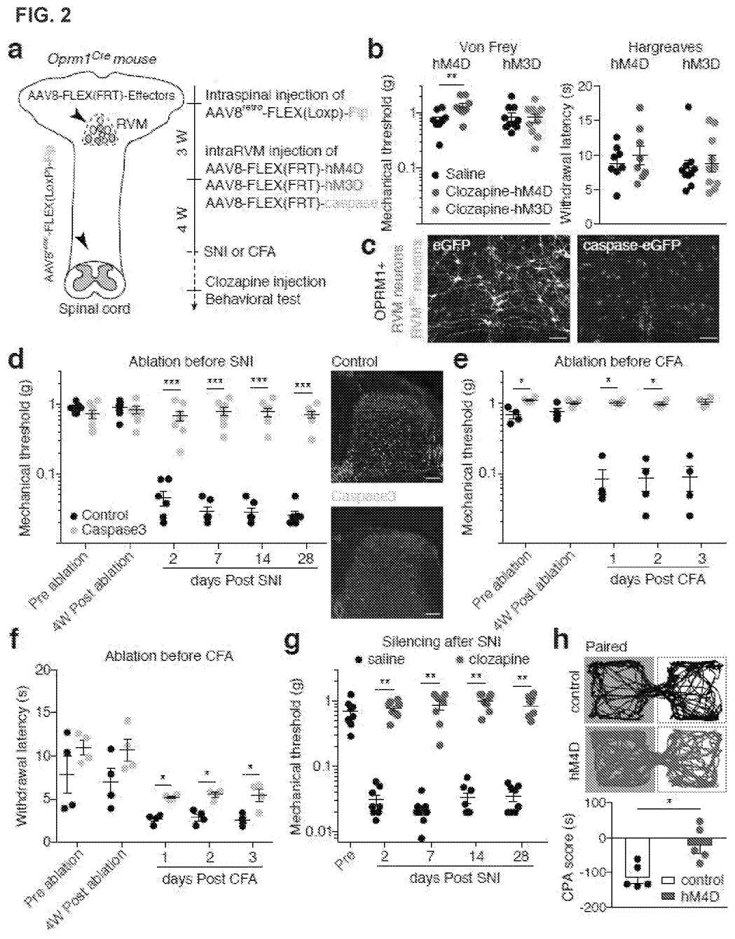

The ability to effectively access the OPRM1+ RVM^{SC} neurons offers the opportunity to determine their roles in normal nociception and persistent pain caused by nerve injury and inflammation. OPRM1+ RVM^{SC} neurons were transduced with inhibitory or excitatory designed receptors hM4Di or hM3Dq21, respectively, so that these neurons could be experimentally activated or inhibited by intraperitoneal (I.P.) injection of clozapine (FIG. 2a)²². The results showed that acute inhibition or activation of OPRM1+ RVM^{SC} neurons in these mice had little effect on their behavioral responses to mechanical and temperature stimuli, indicating that contribution of the descending OPRM1+ pathway to acute, transient nociception is small (FIG. 2b). Roles of OPRM1+ RVM^{SC} neurons in neuropathic and inflammatory pain was next investigated. It was reasoned that if OPRM1+ RVM^{SC} neurons are required for developing persistent pain, then ablation of these neurons before injury should prevent its initiation. Activated Caspase3 was expressed in OPRM1+ RVM^{SC} neurons to induce cell-autonomous apoptosis²³, which eliminated their cell body in the RVM and their terminals in the spinal cord (FIGS. 2c and d, see methods). In these ablated mice, neither SNI nor Complete Freund's Adjuvant (CFA) injection decreased mechanical withdrawal threshold in Von Frey test, and mechanical stimulation no longer caused increased c-Fos expression in the spinal cord (FIGS. 2d and e). Notably, CFA still caused significant thermal hypersensitivity after ablation of OPRM1+ RVM^{SC} neurons (FIG. 2f). Thus, OPRM1+ RVM^{SC} neurons are required for the initiation of SNI and CFA induced mechanical but not thermal hypersensitization.

Similarly, if activity of OPRM1+ RVM^{SC} neurons is required for maintaining the persistent pain state, then silencing these neurons after injury should alleviate ongoing pain. hM4Di was expressed in OPRM1+ RVM^{SC} neurons, and SNI surgery was then performed to induce robust mechanical hypersensitivity in these mice. In contrast to the mild effect of silencing OPRM1+ RVM^{SC} neurons in non-injured mice (FIG. 2b), clozapine infusion completely reversed sensitized mechanical responses to normal level (FIG. 2g). Importantly, 28 days after SNI, the time point when SNI induced mechanical hypersensitivity becomes morphine resistant²⁴, silencing of OPRM1+ RVM^{SC} neurons still robustly alleviated neuropathic pain (FIG. 2g). Moreover, it was found that OPRM1+ RVM^{SC} silencing also blocked mechanical stimuli induced conditioned place aversion (CPA) in SNI mice, indicating the reduction of pain-induced negative affect (FIG. 2h). Together, these results revealed that the descending OPRM1+ pathway is required for both the initiation and maintenance of mechanical hypersensitivity.

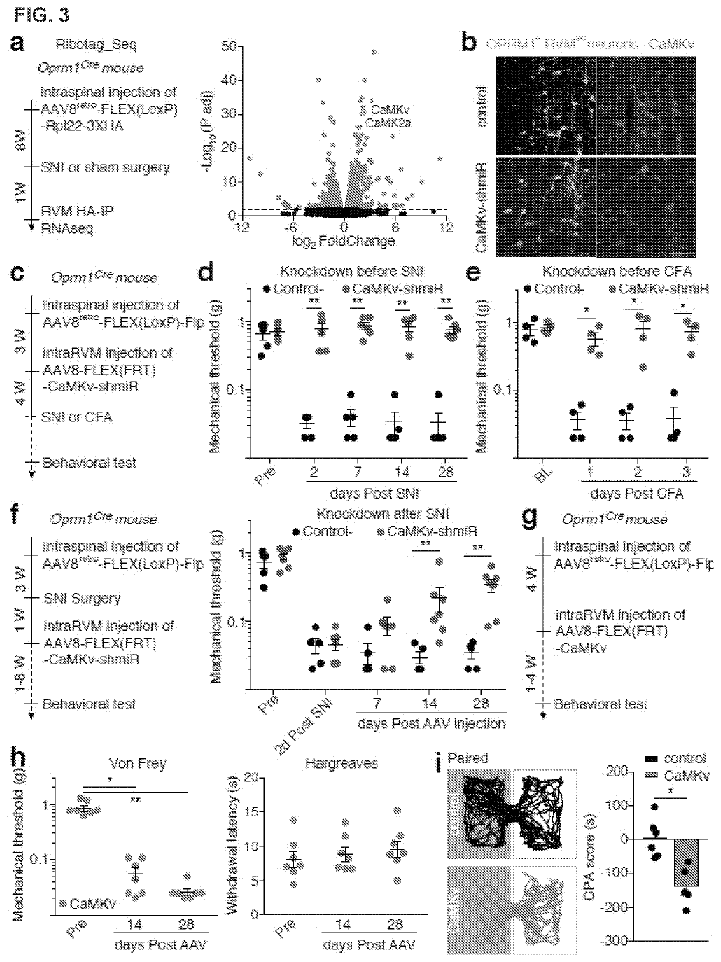

The finding of OPRM1+ RVM^{SC} neurons as a potent target for treating mechanical hypersensitivity prompted further investigation into its underlying molecular mechanisms. Key molecular players should be not only necessary for nerve injury caused mechanical hypersensitivity, but also sufficient to drive mechanical hypersensitivity without injury. To identify such molecules, a Ribosomal Tagging (RiboTag) strategy was used by selectively expressing hemagglutinin A-tagged ribosomal protein L22 (Rpl22-HA) in OPRM1+ RVM^{SC} neurons. Ribosome-associated mRNAs were then immunoprecipitated and sequenced, to profile actively translated genes in these neurons (FIG. 3a, see methods)²⁵. By comparing RiboTag sequencing results from mice with or without SNI surgery, ˜200 genes were identified that showed at least a two-fold difference in their expression levels. Among these differentially expressed genes, two Ca2+/calmodulin-dependent protein kinases were of particular interest, CaMK2a and CaMKv (calmodulin kinase-like vesicle-associated), as their synthesis is regulated by neuronal activity and both of them can play important roles in neuronal plasticity (FIG. 3a)^{26,27}.

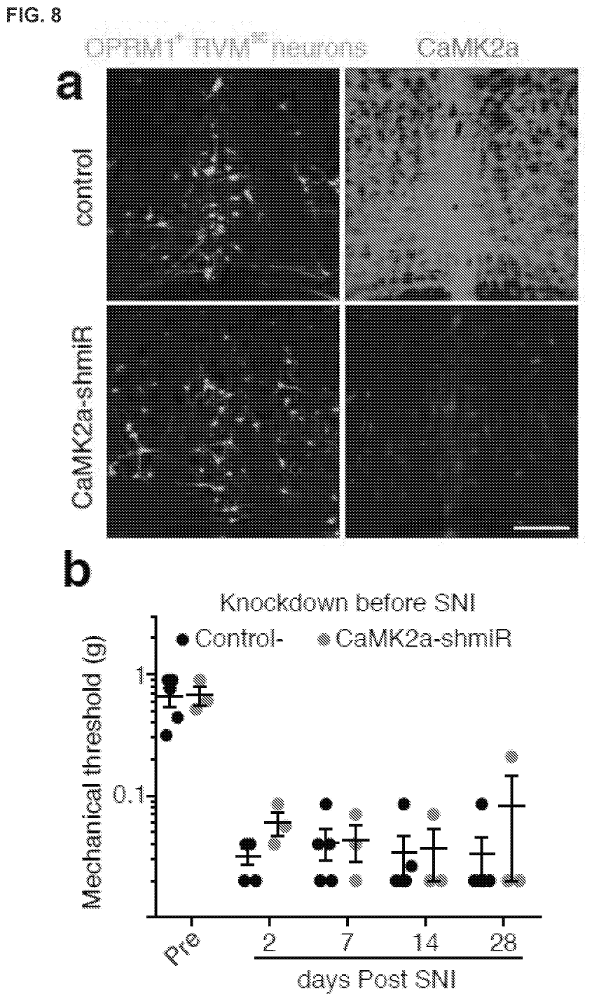

If upregulation of CaMK2a or CaMKv in OPRM1+ RVM^{SC} neurons mediate SNI induced mechanical hypersensitivity, then reducing their expression should alleviate neuropathic pain. To achieve high knockdown efficiency, a shmiR strategy was used by embedding CaMK2a or CaMKv specific short hairpin RNA (shRNA) in the miRNA-155 backbone (FIGS. 3b and c)²⁸. Expression of shmiR for CaMKv but not for CaMK2a in OPRM1⁺ RVM^{SC} neurons before SNI or CFA injection prevented the development of mechanical hypersensitivity (FIGS. 3d and e, FIG. 8). Expression of CaMKv-shmiR after SNI gradually restored mechanical response threshold to normal (FIG. 3f). Therefore, CaMKv is necessary for both the initiation and maintenance of mechanical hypersensitivity after nerve injury. To assess its sufficiency, CaMKv was selectively overexpressed in OPRM1+ RVM^{SC} neurons in normal mice and an increased sensitivity in mechanical but not thermal thresholds was observed in these mice (FIGS. 3g and h). Moreover, repetitive innocuous mechanical stimuli (0.16 g filament) resulted in robust CPA in CaMKv-overexpressing but not control mice (FIG. 3i). Together, the results established a causal role for CaMKv in OPRM1+ RVM^{SC} neurons in mediating hypersensitivity of mechanical pain.

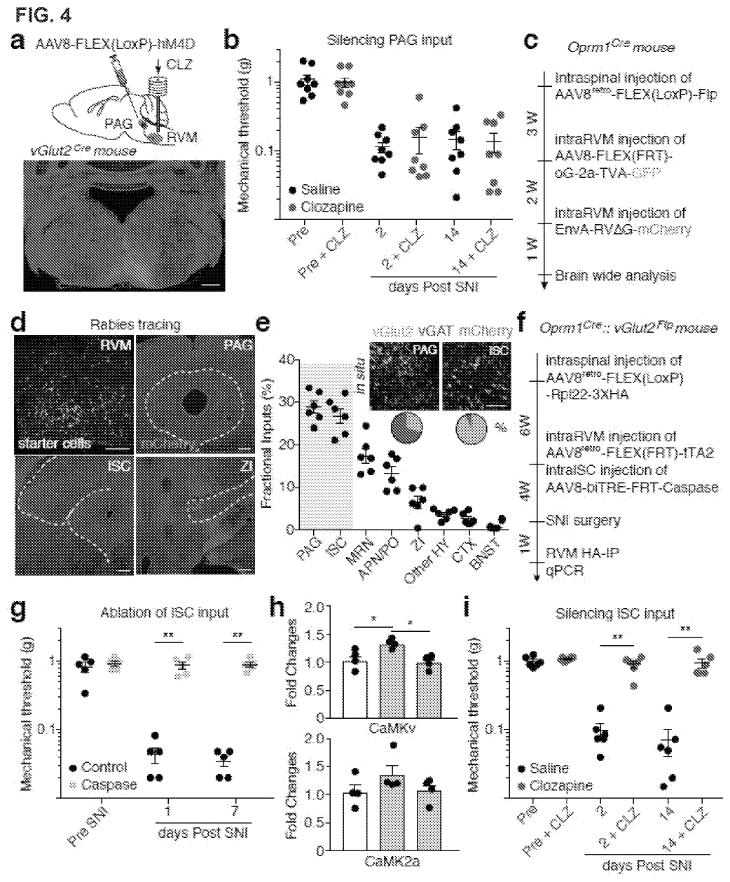

Because increasing neuronal activity promotes CaMKv synthesis and CaMKv is essential for mechanical hypersensitivity, the next task was to identify excitatory inputs onto OPRM1+ RVM^{SC} neurons. It was hypothesized that silencing the excitatory input that drives CaMKv upregulation would not only restore normal levels of CaMKv translation but also alleviate SNI induced mechanical hypersensitivity. Current models emphasize PAG to RVM transmission in descending pain modulation, the impact of silencing the excitatory PAG terminals in the RVM on nociception and mechanical hypersensitivity was therefore examined. AAV8-FLEX(LoxP)-hM4Di was injected into the ventrolateral PAG of vGlut2-Cre mice, then infused clozapine in the RVM to silence the excitatory PAG to RVM pathway (FIG. 4a). Surprisingly, silencing this pathway had no effect on either the baseline mechanical threshold or SNI-induced mechanical hypersensitivity (FIG. 4b), indicating the existence of other excitatory inputs responsible for driving the engagement of OPRM1+ RVM^{SC} neurons in mechanical hypersensitivity.

cTRIO (Tracing the Relationship of Inputs and Outputs) experiments were therefore performed to identify all monosynaptic inputs onto OPRM1+ RVM^{SC} neurons. Codon optimized glycoprotein and EnvA receptor were expressed specifically in the OPRM1+ RVM^{SC} neurons, then EnvA-pseudotyped mCherry expressing G-deleted rabies virus was injected into the RVM (FIG. 4c). Examining mCherry expressing neurons throughout the entire brain, it was found that, after PAG, intermediate/deep gray layers of lateral superior colliculus (lSCIndG) contained the second largest number of retrogradely labeled neurons (FIGS. 4d and e). These brain regions were further examined using RNAscope probing for inhibitory (VGAT) and excitatory (VGLUT2) neurons and found that about 30% of retrogradely labeled neurons in PAG were VGLUT2+, whereas this number is more than 90% in lSCIndG (FIG. 4e, inset). Therefore, lSCIndG provided the most prominent excitatory input onto OPRM1+ RVM^{SC} neurons.

Could excitatory inputs from lSCIndG mediate upregulation of CaMKv in OPRM1+ RVM^{SC} neurons after SNI? To address this question, the impact of ablating this input on the CaMKv synthesis was quantified. In the Oprm1-Cre and vGlut2-Flp double transgenic mice, OPRM1+ RVM^{SC} neurons were transduced with Rpl22-HA, then injected AAV8-retro expressing Flp-dependent tetracycline-controlled transactivator (AAV8-retro-FLEX(FRT)-tTA) into the RVM. In the same mice, AAV8 expressing Flp-dependent Caspase3 under the control of the low-leakiness bidirectional tetracycline-responsive element (AAV8-FLEX(FRT)-biTRE-Caspase3) was injected into lSCIndG to selectively ablate excitatory RVM projecting neurons in the lSCIndG (FIG. 4f, see method). AAV8-FLEX(FRT)-biTRE-BFP was used as control. Four weeks later, it was found that SNI surgery could no longer cause mechanical hypersensitivity in caspase ablated mice, but not BFP expressing control mice (FIG. 4g). After behavioral tests, the RVM from these mice was dissected, their ribosomes were immunoprecipitated, and qPCR was used to quantify ribosome-associated CaMKv mRNA. qPCR analysis revealed that CaMKv translation levels were similar between SNI-caspase and non-SNI groups, but lower than SNI-control group (FIG. 4h). Lastly, chemogenetic silencing of excitatory lSCIndG terminals in RVM after SNI also restored normal mechanical threshold (FIG. 4i). Therefore, lSCIndG inputs are required for SNI induced upregulation of CaMKv translation, as well as for initiation and maintenance of mechanical hypersensitization.

Taken together, the results support a model in which excitatory input from lSCIndG promotes upregulation of protein synthesis of CaMKv in OPRM1+ RVM^{SC} neurons after nerve injury, which drives the initiation and maintenance of mechanical hypersensitivity. Silencing of the excitatory collicular-RVM pathway, inhibitory OPRM1+ RVM-spinal cord pathway, or knocking down CaMKv in OPRM1+ RVM^{SC} neurons, restored normal mechanical responses in SNI mice. These results implicate the collicular-medulla-spinal cord pathway as an attractive target for treating chronic mechanical pain. lSCIndG is known to be important for sensorimotor transformation. It receives both bottom-up inputs from the spinal cord and also top-down inputs from the somatosensory and motor cortex.

The cTRIO tracing and in situ characterization revealed both excitatory and inhibitory PAG neurons innervating OPRM1+ RVM^{SC} neurons. Although silencing of excitatory PAG terminals in the RVM did not affect either nociception or SNI-induced mechanical pain, the inhibitory PAG-RVM pathway can be used to modulate pain. Notably, cTRIO tracing also identified zona incerta, which contains mostly inhibitory neurons, directly innervates OPRM1+ RVM^{SC} neurons (FIG. 4d). Activation of these inhibitory inputs can decrease activity in the descending OPRM1+ pathway and alleviate mechanical pain. Lastly, CaMKv was identified as a key molecular determinant for mechanical pain in OPRM1+ RVM^{SC} neurons. CaMKv binds calmodulin in the presence of calcium but lacks kinase activity, and is hence a pseudokinase.

### Methods

### Mice

All procedures were in accordance with the US National Institutes of Health (NIH) guidelines for the care and use of laboratory animals, and were approved by Stanford University's Administrative Panel on Laboratory Animal Care. Mice (1.5 day-10 weeks) from both genders were used in experiments. Genetically-engineered mouse lines used in this study included Oprm1Cre/+ (see below), Vglut2-IRES-Cre (JAX #016963), Vglut2-IRES2-FLPo-D (JAX #030212). Oprm1Cre/+; Vglut2FLP/+ line was generated by crossing the corresponding lines listed above. The Oprm1Cre/+ knock-in mouse line was generated in the Stanford University Transgenic, Knockout and Tumor model Center (TKTC) using conventional ES cell targeting strategies. The Cre recombinase cDNA, followed by the rabbit β-globin poly-A signal, was introduced via homologous recombination immediately after the start codon in exon 1 of the mouse Oprm1 gene (FIG. 1a). Heterozygous mice were generated by mating chimeric mice to C57BL/6 mice.

### Virus

The following AAV virus were produced and packaged in the lab and used in this study: AAV8-hSyn-H2BClover3-FLEX(LoxP)-H2BmRuby3 (2.0E13 gc ml-1), AAV8-hSyn-FLEX(FRT)-hM4D-IRES-EGFP (2.0E13 gc ml-1), AAV8-hSyn-FLEX(FRT)-hM3D-EYFP (3.0E13 gc ml-1), AAV8-hSyn-FLEX(FRT)-taCaspse3-TEVp (1.0E13 gc ml-1) or AAV8-hSyn-FLEX(LoxP)-Ruby3-FLEx(FRT)-Clover3 (1.0E13 gc ml-1), AAV8-retro-hSyn-FLEX-mTagBFP2-P2A-FlpO (5.0E13 gc ml-1), AAV8-hSyn-FLEX(FRT)-Clover3-shmiRNACamkv (1.0E13 gc ml-1), AAV8-hSyn-FLEX(FRT)-Clover3-shmiRNACamk2a (1.0E13 gc ml-1), AAV8-hSyn-FLEX(FRT)-Clover3-shmiRNAscrambled (1.0E13 gc ml-1), AAV8-retro-hSyn-FLEX(LoxP)-Rpl22-3XHA (2.0 E13 gc ml-1), AAV8-retro-hSyn-FLEX(LoxP)-tTA2 (5.0 E13 gc ml-1), AAV8-hSyn-FLEX(LoxP)-hM4D-IRES-GFP (5.0E13 gc ml-1), AAV8-hSyn-FLEX(FRT)-jGCaMP7S (5.0E13 gc ml-1), AAV8-Ruby3-biTRE-FRT-FLEX(LoxP)-mTagBFP2 (2.0 E13 gc ml-1), AAV8-Ruby3-biTRE-FRT-FLEX(LoxP)-taCaspse3-TEVp (2.0 E13 gc ml-1), AAV8-hSyn-FLEX(FRT)-EGFP-P2A-TVA-T2A-oG (5.0E13 gc ml-1), and SAD□G-mCherry(EnvA) (2.0 E8 IU ml-1).

### Surgery

Stereotaxic injection, optical fiber and cannular implantation. Stereotaxic surgeries were performed on 5- to 7-week old mice under ketamine and xylazine (100 mg Kg-1 and 5 mg Kg-1, i.p.) anesthesia using a stereotaxic instrument (BenchMARK Digital, Lecia). Virus was injected into the RVM (200 nl AAV, bregma −5.60 mm, lateral 0.1 mm, ventral 5.75 mm), lateral SC (200 nl AAV, bregma −3.45 mm, lateral 1.65 mm, ventral 2.40 mm), PAG (250 nl AAV, bregma −4.65 mm, lateral 0.5 mm, ventral 3 mm) with a pulled glass capillary at a slow rate (100 nl min-1) using a pressure microinjector (Micro 4 system, World Precision Instruments). The injection capillary was removed 5 min after the end of the injection. For mice used for chemogenetic silencing or fiber photometry, an infusion cannula (PlasticsOne) or optical fiber (Inper, HangZhou, China) was placed at least 500 μM above the target brain region and cemented to the skull using dental cement (Lang Dental Manufacturing). After surgery, a dummy cannula was inserted and a cap was screwed on to keep the guide cannula from becoming occluded. Mice were allowed at least 2 weeks to recover and to express the virus before behavioral training commenced.

Spared nerve Injury. SNI surgery were performed as previously described on 5- to 7-week old mice under ketamine and xylazine (100 mg Kg−1 and 5 mg Kg−1, i.p.) anesthesia. Briefly, following skin incision and blunt dissection to expose the sciatic nerve, and the tibial and common peroneal branches of the sciatic nerve were ligated with a 5.0 silk suture and transected distally, while the sural nerve was left intact. For sham surgery, only skin incision and blunt dissection were performed. After injury, skin was sutured, and mice were recovered on heated pad before being returned to their home cage. Mechanical and thermal thresholds were measured 2 days after the surgery.

CFA injection. Mice were anesthetized with isoflurane (2%). 5 μl of CFA was injected into the plantar surface of the left hindpaw. Mechanical and thermal thresholds were measured at 1-3 days after CFA treatment.

Neonatal spinal cord injection. The neonatal intraspinal cord injection method was modified based on the published method31. Neonatal pups were injected within 1.5-2.5 days after birth. The pups were covered in aluminum foil then surrounded in ice for 3-4 minutes until all movement stops and the skin color changes from pink to purple. 1-3 μl of rAAV vector containing 0.04% Tyrpan blue (for visualization of the injection site) was slowly injected into the spinal cord of cryoanesthetized neonates using 5 μl syringes (1 inch needle, 30 degrees bevel; Hamilton Company, Reno, NV). Only pups with clear visualization of blue line in the back of the body were used for experiments. After injection, pups were recovered on a warming blanket till the skin color changes to pink with body movements can be observed and then returned to the home cage.

### Fiber Photometry

Fiber photometry experiments were performed at least 4 weeks after AAV8-hSyn-FLEX(FRT)-jGCaMP7S was injected into the RVM of the Oprm1Cre/+ mice receiving neonatal intraspinal cord injection of AAV8-retro-hSyn-FLEX(LoxP)-mTagBFP-P2A-FlpO. The implanted fiber was connected to Fiber Optic Meter (FOM-02M, C-Light, SooChow, China) through an optical fiber patch cord (400 μm, 0.50 NA, Inper, Hangzhou, China). Mice were habituated for 3 days (30 min each) to fiber tethering before fiber photometry recording. To record fluorescence signals, a beam from a 480 LED was reflected with a dichroic mirror, focused with a lens coupled to an CMOS detector (Thorlabs, Inc. DCC3240M). The LED power at the tip of the patch cord was less than 50 μW. A Labview program was used to control the CMOS camera which recorded calcium signals at 50 Hz. The analog voltage signal was digitalized, filtered (200 Hz low-pass) and sampled at 3 kHz using a RZ5D processor (Tucker-Davis Technologies). Fiber photometry data were recorded using OpenEx software (Tucker-Davis Technologies) and analyzed using custom-written MATLAB script (MathWorks). To record mechanical stimulation evoked response in uninjured and SNI mice, 4 g and 0.4 g von Frey fiber was applied to the lateral part of the plantar surface of the hind paw for 4-6 times with a 30 sec interval between each stimulation, respectively. For thermal stimulation, an infrared laser (70% of maximum power, Ugo Basile SRL) or dry ice was applied to the plantar surface through a glass pane underneath of the hind paw for 4-6 times with a 30 sec interval between each stimulation. The video recording was synchronized with the fiber photometry recording through a TL signal. The fluorescence change (ΔF/F) was calculated as (F−F0)/F0, where F0 is averaged fluorescence signals during 3 seconds baseline period in each trial.

### Chemogenetic Manipulation

For chemogenetic activation or silencing experiments, 0.1 mg Kg−1clozapine were injected (i.p.) 30 mins before behavior tests. For terminal silencing experiments, 300 nl of 5 μM clozapine were infused into the RVM through the cannula 30 mins before behavior tests.

### Behavior Tasks

Von Frey Test. Each mouse was habituated in a red plastic cylinder on an elevated wire grid for at least 1 h prior to testing. Mechanical sensitivity was determined with a set of calibrated von Frey filaments (0.02-4 g). For SNI model, filament was applied to lateral part of the left hindpaw. Between individual measurements, von Frey filaments were applied at least 3s after the mice had returned to their initial resting state. The 50% paw withdrawal threshold was determined using the Dixon's up-down method32.

Hargreaves' Test. Each mouse was habituated in a red plastic cylinder on a glass floor for at least 1 h prior to testing. A radiant heat beam (Hargreaves apparatus, Ugo Basile) was focused onto the left hind paw. The latency to hindpaw withdrawal was recorded with at least 2 trials per animal repeated ≥10 min apart. A cut-off latency of 20 s was set to avoid tissue damage.

Conditioned place aversion (CPA) test. The CPA assay was used based on previously described 33. Briefly, mice were habituated for 3 days (30 min each) to a custom-designed two-compartment CPA apparatus (30 cm×25 cm×20 cm) placed on an elevated mesh rack. Each chamber contained unique visual cues (black and grey stripes or plain grey walls). On the final day of habituation, baseline preferences were video-recorded for 10 min and movement was tracked using the custom tracking software running on MATLAB (MathWorks). Following baseline measurements, animals were confined to their preferred side of the chamber and paired with repeatedly stimulating the left hindpaw once every 10 s for 10 min using a 0.16 g filament. After pairing, mice were returned to their home cage for 20 min before re-exposed to the CPA chamber with free access to both side of the CPA chamber for 10 min. CPA scores were calculated by subtracting the time spent in the filament stimulation-paired side of the chamber during baseline from the time spent in the same side of the chamber during the re-exposure.

### Ctrio Tracing and Analysis.

Experiments in FIG. 4c-e were performed in Oprm1Cre/+ mice with neonatal injection of AAV8-retro-hSyn-FLEX(LoxP)-mTagBGFP-P2A-FlpO in the spinal cord. Six weeks later, 200 nl of AAV8-hSyn-FLEX(FRT)-EGFP-P2A-TVA-T2A-oG were injected into the RVM using a stereotaxic instrument (BenchMARK Digital, Lecia) of mice anesthetized by ketamine and xylazine (100 mg Kg−1 and 5 mg Kg−1, i.p.). Four weeks later, 200 nl SADOG-mCherry(EnvA) was injected into the same area of the RVM using the procedure described above. Mice were housed in a biosafety-level-2 (BSL2) facility for 7 days before sacrificing. For quantification of long-range input brain region, brain regions that are 1 mm anterior or posterior to the injection site were excluded from analysis. Images were taken from consecutive 50 m coronal sections using Zeiss Axioplan2 using 2.5× or 5× objective. Cell counting was performed manually using Fiji. For quantifications of subregions, boundaries were based on the Allen Institute's reference atlas (https://mouse.brain-map.org/experiment/thumbnails/100048576?image_type=atlas). Potential double counting cells from consecutive sections was not adjusted.

### Ablation of Oprm1+ RVM^{SC} Neurons

AAV8-hSyn-FLEX(FRT)-taCaspse3-TEVp and AAV8-hSyn-FLEX(LoxP)-Ruby3-FLEX(FRT)-Clover3 was co-injected into the RVM of the Oprm1Cre/+ mice with neonatal injection of AAV8-retro-hSyn-FLEX-mTagBFP-P2A-FlpO (1 l) in the spinal cord. For control mice, AAV8-hSyn-FLEX(LoxP)-Ruby3-FLEX(FRT)-Clover3 was injected into the RVM. The Oprm1+ RVM neurons express mRuby3, and Oprm1+ RVM^{SC} neurons express both mRuby3 and Clover3. Four weeks later, after behavioral test, the brains and spinal cord were collected from both groups for histological analysis.

### Ribosomal Immunoprecipitation

Isolation of polysome-associated mRNA using RiboTag was performed as previously published34. Briefly, 1.5 mm brainstem region containing the RVM were dissected with surgical scissors. Tissues from 4 mice (control or after SNI) were pooled and transferred into dounce homogenizer containing 1 ml homogenization buffer (50 mM Tris, pH 7.5, 100 mM KCl, 12 mM MgCl2, 1% Nonidet P-40, 1 mM dithiothreitol (DTT), 200 U/mL RNasin, 1 mg/mL heparin, 100 μg/mL cycloheximide, and 1×protease inhibitor mixture) and mechanically dissociated using pestle A and B (15 time each). Lysates were centrifugated for 10 min at 10,000 rpm at 4° C. 80 μl input samples were mixed with 350 μl RLT buffer from the RNeasy Mini Kit (Qiagen) supplemented with beta mercaptoethanol (10 μl/ml) and stored in −80° C. 800 μl supernatants were transferred to fresh 1.5 mL microcentrifuge tubes. For immunoprecipitation, 5 μL of anti-hemagglutinin 1.1 antibody (BioLegend) was added to the lysate-containing tube and incubated for 4 h at 4° C. on a microtube rotator. Pierce™ Protein A/G Magnetic Beads (Thermo Fisher Scientific) were added to the lysate with antibody and incubated on a microtube rotator at 4° C. overnight. After incubation, the microcentrifuge tubes were placed into the magnetic stand on ice to remove the supernatant. The magnetic beads were washed with high-salt buffer (50 mM Tris, pH 7.5, 300 mM KCl, 12 mM MgCl2, 1% Nonidet P-40, 1 mM DTT, and 100 μg/mL cycloheximide) for 3 times to remove the non-specific binding from immunoprecipitation. After washing, 350 μL of RLT plus buffer with p-mercaptoethanol from the RNeasy Mini Kit (Qiagen, Germany) was added. The extraction of total RNA was performed with the RNeasy Mini Kit. All RNA samples were quantified with the Qubit RNA Assay Kit (Invitrogen).

### Ribotag RNA-Seq Library Construction and Analysis

The Ribotag RNA-Seq library was generated by adapting the low input cDNA synthesis method described previously with dual indexing by tagmentation35. 10 ng of RNA was used as input for reverse transcription with an oligo dT primer (E3V6NEXT without bar code or UMI sequence) and TSO (E5V6NEXT) by Maxima H Minus RT. The samples were then bead purified and amplified using a primer complimentary (SINGV6) to the TSO sequence for 8 PCR cycles with Terra polymerase. Samples were then bead purified and dual indexing was performed using the NEXTERA XT kit. The sequencing used performed by the Stanford Genome Sequencing Service Center on the HiSeq 4000 paired end 100 bp run. Analysis of the HiSeq data was performed using standard programs with default settings. Briefly, reads were trimmed and checked with FastQC. Alignment to the mouse genome (GRCm38) was performed with HISAT2. Stringtie was used to assemble and generate counts. Differential expression analysis and initial visualization was performed with DeSeq2.

Quantitative RT-PCR Analysis from Oprm1+ RVMs Neurons.

The ribosome-bound mRNA was isolated from animals with or without SNI, and from SNI animals with the lSCIndG excitatory input ablation. The extraction of total RNA was performed with the RNeasy Mini Kit (Qiagen, Germany), and reverse transcribed into cDNA using Maxima H Minus Reverse Transcriptase (Thermo Fisher). qPCR analysis of CaMKv, CaMK2a, actin was performed using the following primers: 5′ GATGGAGGTGGAGCAAGACC (CaMKv forward), 5′ cggacagccttcttccactt (CaMKv forward); 5′GAAGATGTGCGACCCTGGAA (CaMK2a forward), 5′ TGCGGATATAGGCGATGCAG (CaMK2a reverse); 5′GTGGTACGACCAGAGGCATAC (beta actin forward), 5′ AAGGCCAACCGTGAAAAGAT (beta actin reverse).

### Immunostaining and RNAscope In Situ Hybridization.

Mice were deeply anaesthetized with pentobarbital sodium solution and transcardially perfused with PBS, followed by 4% paraformaldehyde (PFA) in 1×PBS at room temperature. Brains and spinal cords were dissected from perfused mice and post-fixed in 4% PFA in 1×PBS at 4° C. overnight, cryoprotected in 30% sucrose in 1×PBS at 4° C. for overnight, embedded in OCT compound, and frozen using dry ice and kept at −80° C. Brains and spinal cords were cryosectioned (15 m for RNAscope in situ hybridization or 50 m for immunostaining) using a cryostat (Leica). For immunostaining, 50 m sections were washed three times for 5 min each with 1×PBS containing, incubated with blocking solutions (0.3% PBST containing 10% normal donkey serum (LAMPIRE Biological Products 7332100) for 2 h at room temperature, followed by primary antibodies diluted in 0.3% PBST containing 3% normal donkey serum at 4° C. overnight. Sections were washed three times for 10 min each with PBS, incubated with secondary antibodies diluted in 0.3% PBST containing 5% normal donkey serum for 2 h at room temperature, followed by 3 time of washes for 10 min each with PBS (Hoechst 33342 solution, ThermoFisher) was included in the second wash at 1:10,000 dilution) and mounted with Fluoromount-G (Southern Biotech). Primary antibodies used in this study include rabbit anti-mCherry (1:1,000, 600401397, Rockland), rabbit anti-CaMKv (1:200, 147881AP, Proteintech), rabbit anti-cFos (1:1,000, 226003, Synaptic systems), mouse anti-HA (1:1000, 901514, Biolegend). Secondary antibodies included Alexa 594 or 647 conjugated donkey anti-rabbit antibodies, Alexa 647 conjugated donkey anti-mouse antibodies. All secondary antibodies were purchased from Life Technologies and used at 1:1000 dilutions. For RNAscope in situ hybridization, 15 μm sections were collected on glass slide, mRNA transcripts were detected using the RNAscope Fluorescent Multiplex Assay (Advanced Cell Diagnostics) and RNAscope Fluorescent Multiplex Reagent Kit v2 (cat. no. 323100). The RNAscope catalogue probes were used to detect Oprm1 (cat. no. 493251), vgat (cat. no. 319191), vglut2 (cat. no. 319171) ), Tph2 (cat. no. 318691) RNA molecules. Images were obtained using a Zeiss 710 confocal microscope using either 10× (Plan-Apochromat 10×, NA 0.45) or 20× (Plan-Apochromat 20×, NA 0.8) objectives. For imaging large spinal cord and brain sections, the tile-scan function was used and the tile images were stitched using Zeiss Zen microscope software.

For Fos Immunostaining, each SNI mouse was habituated in a red plastic cylinder on a glass floor for at least 1 h prior to testing. The left hindpaw were repeatedly stimulated once every 10 s for 10 min using a 0.16 g filament. 1.5 h after the delivery of each stimulation, mice were perfused and processed for Fos immunohistochemical analysis.

### 2D Registration of RVM Descending Neurons and 3D Visualization

30-40 brain slices (50 μm) containing the RVM were scanned using Olympus VS120 for 2D registration. Custom MATLAB software were used to remove all image features outside the brain slices. Background subtraction and contrast enhancement of the NTB channel were then applied. The processed NTB images for each section were then serially analyzed using a combination of automated and manual methods. For a more detailed description of this procedure see Xiong et al., 201836.

### Statistical Analysis

No statistics were used to predetermine sample size. However, the sample sizes were similar on previously published studies. Statistical methods are indicated when used. All analyses were performed using Prism (GraphPad software). No method of randomization was used in any of the experiments. Experimenters were not blind to group allocation in behavioral experiments, but CPA score were measured automatically by custom tracking software running on MATLAB (MathWorks). All animals that finished the entire behavioral training and testing were included in analysis. Data are presented as mean±SEM.

### Example 2: Addition Experiment Demonstrating Retro-AAV8 Activity

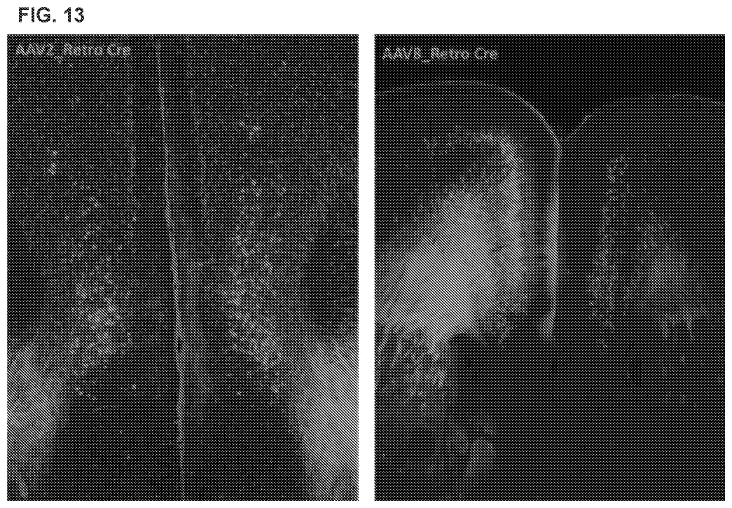

AAV2-retro-Cre and AAV8-retro-Cre were injected into the thalamus of a Ai9 reporter mice to retrogradely label corticothalamic projection neurons. Same titer AAV8-retro-Cre retrogradely labeled 5 times more neurons than AAV2-retro-Cre. (FIG. 13) (Blue are pan neuronal marker Neun, red are RFP signal for retrogradely labeled neurons)

### Example 3: Using AAV to Deliver an RNAi Agent to Knock Down CaMKv

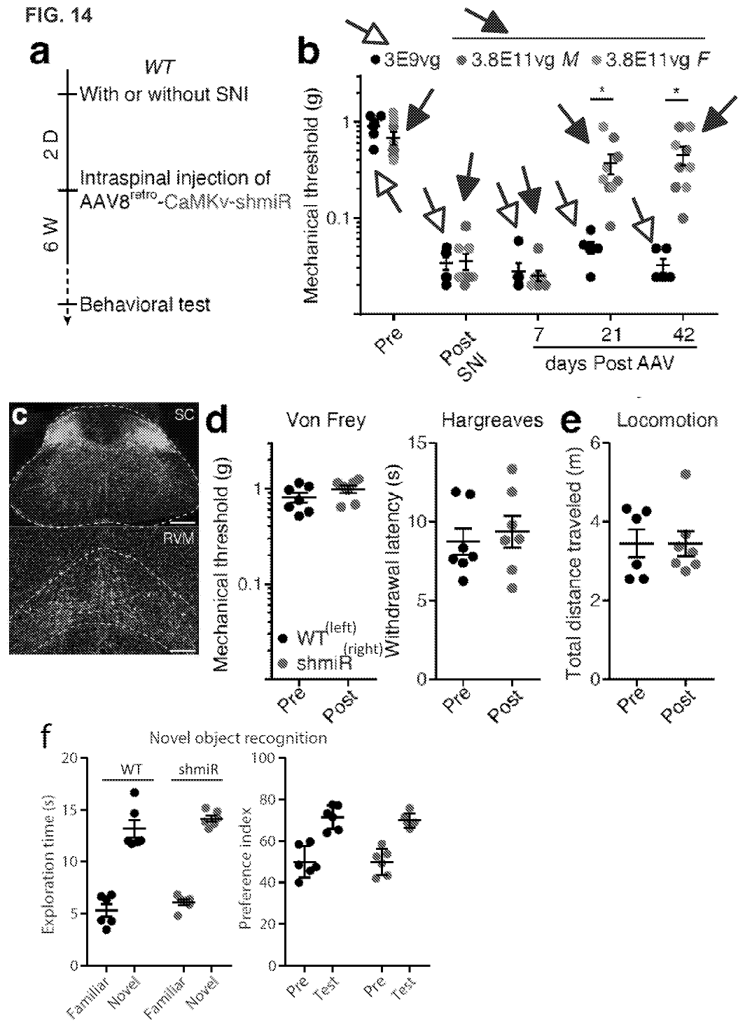

AAV8-retro expressing CaMKv-shmiR for gene therapy of neuropathic pain was further tested. A recent genome-wide association study using UK Biobank reported strong association of SNPs in CaMKv with chronic pain, which further supports our findings that CaMKv is relevant to chronic pain. However, the targeting strategy used above involves the injection of AAV8-retro-FLEX(LoxP)-Flp into the spinal cord and AAV8-FLEX(FRT)-CaMKv-shmiR into the RVM of Oprm1-Cre mice to achieve cell type and pathway specific gene manipulation, which approach would not typically be used in the clinic. To knock down CaMKv in a setting more directly similar to a clinical setting, single intraspinal injection of AAV8-retro-CaMKv-shmiR was performed in adult wild type mice (FIG. 14a). SNI surgery was performed and robust mechanical sensitization in these mice was confirmed using Von Frey test. 1 μl of AAV8-retro-CaMKv-shmiR containing 3.0E9 or 3.8E11 viral particles was injected into the spinal cord of different groups of SNI-mice, respectively. Gradual reduction of mechanical pain was observed over the course of virus expression in mice injected with 3.8E11 but not 3.0E9 viral particles, and the therapeutical effect reached a plateau after about 3 weeks post-virus injection (FIG. 14b). Both male and female mice were used in this experiment and it was found that intraspinal injection of the higher dose of AAV8-retro-CaMKv-shmiR was effective in treating SNI-induced mechanical pain in both sexes of mice (FIG. 14b).

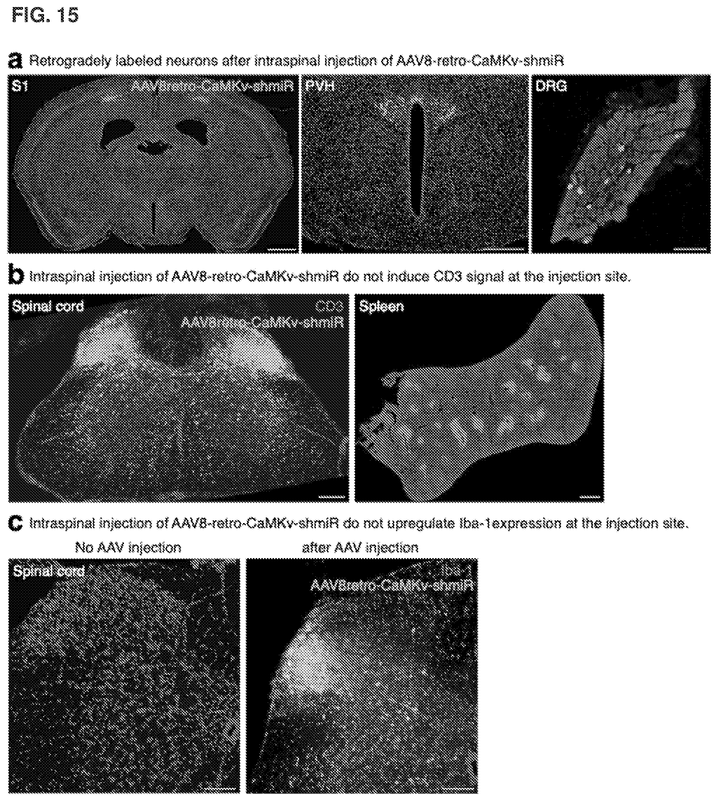

The safety of intraspinal injection of high dose of AAV8-retro-CaMKv-shmiR was next evaluated. As expected, this procedure inevitably led to the expression of CaMKv-shmiR in both spinal neurons at the injection site and spinal cord projecting neurons beyond RVM, such as somatosensory cortex, hypothalamus, as well as sensory neurons in the dorsal root ganglion (FIG. 14c, FIG. 15a). A series of behavioral tests was run to determine whether such broader knockdown of CaMKv caused any adverse side effects. Mechanical and thermal thresholds as well as locomotor activity were measured, and no difference between wild type mice and mice with intraspinal injection of 3.8E11 viral particles of AAV8-retro-CaMKv-shmiR was found (FIG. 14d, FIG. 14e). A novel object recognition task was then performed to test their cognitive function and no difference between these two groups of mice was found (FIG. 14f). Finally, expression of CD3 and iba1, the wildly used marker for infiltrating T cells and microglia activation, respectively, was evaluated. No evidence of immune response and inflammation caused by virus injection and expression was found (FIG. 15b, FIG. 15c). Thus, intraspinal cord injection of high dose AAV8-retro-CaMKv-shmiR into adult wild type mice was a safe and effective approach for treating SNI-induced chronic mechanical pain.

The disclosures of each and every patent, patent application, and publication cited herein are hereby incorporated herein by reference in their entirety. While this disclosure has been with reference to specific embodiments, it is apparent that other embodiments and variations may be devised by others skilled in the art without departing from the true spirit and scope of the disclosure. The appended claims are intended to be construed to include all such embodiments and equivalent variations. In addition, many modifications may be made to adapt a particular situation, material, composition of matter, process, process step or steps, to the objective, spirit and scope of the present disclosure. All such modifications are intended to be within the scope of the claims appended hereto.

What is claimed is:

1. A retrograde-enhanced clade E variant adeno-associated virus (AAV) capsid protein, comprising the amino acid sequence LADQDYTKTA (SEQ ID NO: 30) inserted into a clade E AAV capsid protein.

2. The retrograde-enhanced clade E variant AAV capsid protein of claim 1, wherein the LADQDYTKTA (SEQ ID NO: 30) sequence immediately follows a QQQN (SEQ ID NO: 28), QQTN (SEQ ID NO: 29), QQQD (SEQ ID NO: 30), or QQAN (SEQ ID NO: 31) sequence.

3. The retrograde-enhanced clade E variant AAV capsid protein of claim 1, comprising an amino acid sequence having 85% or more sequence identity with the amino acid sequence set forth in any one of SEQ ID NOs: 32-57.

4. (canceled)

5. The retrograde-enhanced clade E variant AAV capsid protein of claim 1, comprising the amino acid sequence LADQDYTKTA (SEQ ID NO: 30) inserted into an AAV8 capsid protein or into an rh10 capsid protein.

6. (canceled)

7. A transduction system comprising one or more nucleic acids, where said one or more nucleic acids comprises a nucleotide sequence that encodes the retrograde-enhanced clade E variant AAV capsid protein of claim 1.

8. The transduction system of claim 7, where said one or more nucleic acids further comprises a transgene sequence flanked by inverted terminal repeat sequences (ITRs), wherein the transgene sequence encodes a non-coding RNA or a polypeptide.

9. (canceled)

10. The transduction system of claim 8, wherein the non-coding RNA is a short hairpin RNA (shRNA) that targets CamKv or a CRISPR/Cas guide RNA that targets CamKv.

11-14. (canceled)

15. A retrograde-enhanced recombinant AAV particle comprising:
(a) the retrograde-enhanced clade E variant AAV capsid protein of claim 1; and
(b) a nucleic acid comprising a transgene sequence.

16. The retrograde-enhanced recombinant AAV particle of claim 15, wherein the transgene sequence encodes a non-coding RNA or a polypeptide.

17. The retrograde-enhanced recombinant AAV particle of claim 16, wherein the non-coding RNA is a short hairpin RNA (shRNA) that targets CamKv or a CRISPR/Cas guide RNA that targets CamKv.

18-22. (canceled)

23. A method of making a retrograde-enhanced recombinant AAV particle, the method comprising: introducing the transduction system of claim 7 into a eukaryotic cell, wherein the eukaryotic cell produces said retrograde-enhanced recombinant AAV particle.

24. A method of expressing a transgene of interest in a neuron, the method comprising: contacting the neuron with the retrograde-enhanced recombinant AAV particle of claim 15.

25. The method of claim 24, wherein said contacting occurs in an individual's spinal cord or thalamus.

26. The method of claim 24, wherein the neuron is a spinal cord projecting neuron, a spinal cord-projecting neuron of the rostral ventromedial medulla (RVM), a spinal cord-projecting neuron of the locus coeruleus (LC), or a corticothalamic projecting neuron.

27-28. (canceled)

29. The method of claim 24, wherein the neuron is an opioid receptor mu 1 (OPRM1) expressing neuron.

30. A method of treating an individual in need, the method comprising:
administering to an individual who has chronic pain a therapy that reduces CamKv activity in opioid receptor mu 1 (OPRM1) expressing neurons of the individual's rostral ventromedial medulla (RVM).

31. The method of claim 30, wherein said therapy is an agent that reduces expression of CamKv in said neurons.

32-48. (canceled)

49. The method of claim 30, wherein said therapy comprises reducing excitatory input into the RVM from RVM projecting lateral superior colliculus (lSCIndG) neurons.

50. The method of claim 30, wherein said therapy comprises increasing inhibitory input into the RVM.

51. The method of claim 50, wherein said inhibitory input into the RVM is from zona incerta neurons.

52. The method of claim 51, wherein the therapy comprises deep brain stimulation of said zona incerta neurons.


SEQUENCE LISTING (65 sequences)

SEQ ID NO: 1 - protein, 10 aa, synthetic construct
LADQDYTKTA

SEQ ID NO: 2 - protein, 738 aa, adeno-associated virus type 8
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 3 - protein, 738 aa, adeno-associated virus
MAADGYLPDWLEDNLSEGIREWWDLKPGAPKPKANQQKQDDGRGLVLPGCKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEPSPQRSPDSSTGIGKTGQQPAKKRLNFGQTGDSESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTQNEGTKTIANNLTSTIQVFTDSEYQLPYVLGSAHQGCPPPFPADVFMIPQYGYLTLNNGSQAVGRSSFYCLEYFPSQMRRTGNNFEFSYQFEDVPFHSSYAHSQSLDRLMNPLIDQYLYYLSRTQSTGGTAGTQQLLFSQAGPNNMSAQAKNWLPGPCYRQQRVSTTLSQNNNSNFAWTGATKYHLNGRDSLVNPGVAMATHKDDEERFFPSSGVLMFGKQGAGKDNVDYSSVMLTSEEEIKTTNPVATEQYGVVADNLQQQNAAPIVGAVNSQGALPGMVWQNRDVYLQGPIWAKIPHTDGNFHPSPLMGGFGLKHPPPQILIKNTPVPADPPTTFSQAKLASFITQYSTGQVSVEIEWELQKENSKRWNPEIQYTSNYNKSVNVDFTVDTNGVYSEPRPIGTRYLTRNL

SEQ ID NO: 4 - protein, 738 aa, adeno-associated virus
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEPSPQRSPDSSTGIGKTGQQPAKKRLNFGQTGDSESVPDPQPIGEPPAGPSGLGSGTMAAGGGAPMADNNEGADGVGSSSGNWHCDSAWLGDRVITTSTRPWALPTYNNHLYKQISNGTSGGSTNDNTYFGYSTPWGYFDFNRFHCHFSPRDWQRLINNNWGFRPKRLNFKLFNIQVKEVTQNEGTKTIANNLTSTIQVFTDSEYQLPYVLGSAHQGCPPPFPADVFMIPQYGYLTLNNGSQAVGRSSFYCLEYFPSQMRRTGNNFEFSYQFEDVPFHSSYAHSQSLDRLMNPLIDQYLYYLSRTQSTGGTAGTQQLLFSQAGPNNMSAQAKNWLPGPCYRQQRVSTTLSQNNNSNFAWTGATKYHLNGRDSLVNPGVAMATHKDDEERFFPSSGVLMFGKQGAGKDNVDYSSVMLTSEEEIKTTNPVATEQYGVVADNLQQQNAAPIVGAVNSQGALPGMVWQNRDVYLQGPIWAKIPHTDGNFHPSPLMGGFGLKHPPPQILIKNTPVPADPPTTFSQAKLASFITQYSTGQVSVEIEWELQKENSKRWNPEIQYTSNYYKSTNVDFAVNTEGTYSEPRPIGTRYLTRNL

SEQ ID NO: 5 - protein, 738 aa, adeno-associated virus
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEAAKTAPGKKRPVEPSPQRSPDSSTGIGKKGQQPAKKRLNFGQTGDSESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYTFEDVPFHSSYAHSQSLDRLMNPLIDQYLYYLSRTQSTGGTQGTQQLLFSQAGPANMSAQAKNWLPGPCYRQQRVSTTLSQSNNSNFAWTGATKYHLNGRDSLVNPGVAMATHKDDEERFFPSSGVLMFGKQGAGRDNVDYSSVMLTSEEEIKTTNPVATEQYGVVADNLQQTNTGPIVGNVNSQGALPGMVWQNRDVYLQGPIWAKIPHTDGNFHPSPLMGGLGLKHPPPQILIKNTPVPADPPTTFSQAKLASFITQYSTGQVSVEIEWELQKENSKRWNPEIQYTSNYYKSTNVDFAVNTEGTYSEPRPIGTRYLTRNL

SEQ ID NO: 6 - protein, 738 aa, adeno-associated virus
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEAAKTAPGKKRPVEPSPQRSPDSSTGIGKKGQRPAKKRLNFGQTGDSESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLSFKPFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYTFEDVPFHSSYAHSQSLDRLMNPLIDQYLHYLSRTQSTGGTQGTQQLLFSQAGPANMSAQAKNWLPGPCYRQQRVSTTLSQNNNSNFAWTGATKYHLNGRDSLVNPGVAMATHKDDEERFFPSSGVLMFGKQGAGRDNVDYSSVMLTSEEEIKTTNPVATEQYGVVADNLQQTNTGPIVGNVNSQGALPGMVWQNRDVYLQGPIWAKIPHTDGNFHPSPLMGGFGLKHPPPQILIKNTPVPADPPTTFSQAKLASFITQYSTGQVSVEIEWELQKENSKRWNPEIQYTSNYYKSTNVDFAVNTEGTYSEPRPIGTRYLTRNL

SEQ ID NO: 7 - protein, 738 aa, adeno-associated virus
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEAAKTAPGKKRPVEPSPQRSPDSSTGIGKKGQQPAKKRLSFGQTGDSESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNSEFSYTFEDVPFHSSYAHSQSLDRLMNPLIDQYLYYLSRTQSTGGTQGTQQLLFSQAGPANMSAQAKNWLPGPCYRQQRVSTTLSQNNNSNFAWTGATKYHLNGRDSLVNPGVAMATHKDDEERFFPSSGVLMFGKQGAGRDNVDYSSVMLTSEEEIKTTNPVATEQYGVVADNLQQTNTGPIVGNVNSQGALPGMVWQNRDVYLQGPIWAKIPHTDGNFHPSPLMGGFGLKHPPPQILIKNTPVPADPPTTFSQAKLASFITQYSTGQVSVEIEWELQKENSKRWNPEIQYTSNYYKSTNVDFAVNTEGTYSEPRPIGTRYLTRNL

SEQ ID NO: 8 - protein, 738 aa, adeno-associated virus
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEAAKTAPGKKRPVEPSPQRSPDSSTGIGKKGQQPAKKRLNFGQTGDSESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYTFEDVPFHSSYAHSQSLDRLMNPLIDQYLYYLSRTQSTGGTQGTQQLLFSQAGPANMSAQAKNWLPGPCYRQQRVSTTLSQNNNSNFAWTGATKYHLNGRDSLVNPGVAMATHKDDEERFFPSSGVLMFGKQGAGRDNVDYSSVMLTSEEEIKTTNPVATEQYGVVADNLQQTNTGPIVGNVNSQGALPGMVWQNRDVYLQGPIWAKIPHTDGNFHPSPLMGGFGLKHPPPQILIKNTPVPADPPTTFSQAKLASFITQYSTGQVSVEIEWELQKENSKRWNPEIQYTSNYYKSTNVDFAVNTEGTYSEPRPIGTRYLTRNL

SEQ ID NO: 9 - protein, 738 aa, adeno-associated virus
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGPVEEAAKTAPGKKRPVEPPPQRSPDSSTGIGKKGQQPAKKRLNFGQTGDSESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLSFKLFNIQVKEVTQNEGTKTVANNLTSTIQVFTDSEYQLPYVLGSAHQGCLPPFPADVFMIPQYGYLTLNNGSQAVGRSSFYCLEYFPSQMLRTGNNFEFSYTFEDVPFHSSYAHSQSLDRLMNPLIDQYLYYLSRTQSTGGTQGTQQLLFSQAGPANMSAQAKNWLPGPCYRQQRVSTTLSQNNNSNFAWTGATKYHLNGRDSLVNPGVAMATHKDDEERFFPSSGVLMFGKQGAGRDNVDYSSVMLTSEEEIKTTNPVATEQYGVVADNLQQTNTGPIVGNVNSQGALPGMVWQNRDVYLQGPIWAKIPHTDGNFHPSPLMGGFGLKHPPPQILIKNTPVPADPPTTFSQAKLASFITQYSTGQVSVEIEWELQKENSKRWNPEIQYTSNYYKSTNVDFAVNTEGTYSEPRPIGTRYLTRNL

SEQ ID NO: 10 - protein, 738 aa, adeno-associated virus
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEAAKTAPGKKRPVEPSPQRSPDSSTGIGKKGQQPAKKRLNFGQTGDSESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLSFKLFNIQVKEVTQDEGTKTIANNLTSTIQVFTDSEYQLPYVLGSAHQGCLPPFPADVFMIPQYGYLTLNNGSQAVGRSSFYCLEYFPSQMLRTGNNFEFSYTFEDVPFHSSYAHSQSLDRLMNPLIDQYLYYLSRTQSTGGTQGTQQLLFSQAGPANMSARAKNWLPGPCYRQQRVSTTLSQNNNSNFAWTGATKYHLNGRDSLVNPGVAMATHKDDEERFFPSSGVLMFGKQGAGRDNVDYSSVMLTSEEEIKTTNPVATEQYGVVADNLQQANTGPIVGNVNSQGALPGMVWQNRDVYLQGPIWAKIPHTDGNFHPSPLMGGFGLKHPPPQILIKNTPVPADPPTTFSQAKLASFITQYSTGQVSVEIEWELQKENSKRWNPEIQYTSNYYKSTNVDFAVNSEGTYSEPRPIGTRYLTRNL

SEQ ID NO: 11 - protein, 731 aa, adeno-associated virus
MAADGYLPDWLEDNLSEGIREWWALKPGAPQPKANQQKQDDGRGLVLPGYKYLGPFNGLDKGEPVNEADAAALEHDKAYDQQLKAGDNPYLRYNHADAEFQERLQEDTSFGGNLGRAVFQAKKRVLEPLGLVEEGAKTAPGKKRPVEPDSSSGIGKSGQQPAKKRLNFGQTGDSESVPDPQPLSEPPAGPSGLGSGTMAAGGGAPMADNNEGADGVGNVSGNWHCDSTWLGDRVITTSTRTWALPTYNNHLYKQISNGTSGGSSNDNTYFGYSTPWGYFDFNRFHCHFSPRDWQRLINNNWGFRPKKLNFKLFNIQVKEVTQNEGTKTIANNLTSTVQVFTDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLSRTQTNGTNATQTLLFAQAGPQNMSAQAKNWLPGPCYRQQRVSTTVSQNNNSNFTWTGATKYHLNGRDSLVSPGVAMATHKDDEERFFPSSGVLMFGKQGAGKDNVEYTNVMLTSEEEIKTTNPVATEQYGVVADNLQQTNSAPIVGAVNSQGALPGMVWQNRDVYLQGPIWAKIPHTDGNFHPSPLMGGFGLKHPPPQILIKNTPVPADPPVNFTDAKLASFITQYSTGQVSVEIEWELQKENSKRWNPEIQYTSNYYKSANVDFAVNADGVYSEPRPIGTRYLTRNL

SEQ ID NO: 12 - protein, 731 aa, adeno-associated virus
MAADGYLPDWLEDNLSEGIREWWALKPGAPQPKANQQKQDDGRGLVLPGYKYLGPFNGLDKGEPVNEADAAALEHDKAYDQQLKAGDNPYLRYNHADAEFQERLQEDTSFGGNLGRAVFQAKKRVLEPLGLVEEGAKTAPGKKRPVEPDSSSGIGKSGQQPAKKRLNFGPTGDSESVPDPQPLSEPPAGPSGLGSGTMAAGGGAPMADNNEGADGVGNVSGNWHCDSTWLGDRVITTSTRTWALPTYNNHLYKQISNGTSGGSSNDNTYFGYSTPWGYFDFNRFHCHFSPRDWQRLINNNWGFRPKKLNFKLFNIQVKEVTQNEGTKTTANNLTSTVQVFTDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLSRTQTNGTNATQTLLFAQAGPQNMSAQAKNWLPGPCYRQQRVSTAVSQNNNSNFTWTGATKYHLNGRDSLVNPGVAMATHKDDEERFFPSSGVLMFGKQGAGKDNVEYTNVMLTSEEEIKTTNPVATEQYGVVADNLQQTNSAPIVGAVNSQGALPGMVWQNRDVYLQGPIWAKIPHTDGNFHPSPLMGGFGLKHPPPQILIKNTPVPADPPVNFTDAKLASFITQYSTGQVSVEIEWELQKENSKRWNPEIQYTSNYYKSANVDFAVNADGVYSEPRPIGTRYLTRNL

SEQ ID NO: 13 - protein, 731 aa, adeno-associated virus
MAADGYLPDWLEDNLSEGIREWWALKPGAPQPKANQQKQDDGRGLVLPGYKYLGPFNGLDKGEPVNEADAAALEHDKAYDQQLKAGDNPYLRYNHADAEFQERLQEDTSFGGNLGRAVFQAKKRVLEPLGLVEEGAKTAPGKKRPVEPDSSSGIGKSGRQPAKKRLNFGQTGDSESVPDPQPLSEPPAGPSGLGSGTMAAGGGAPMADNNEGADGVGNASGNWHCDSTWLGDRVITTSTRTWALPTYNNHLYKQISNGTSGGSSNDNTYFGYSTPWGYFDFNRFHCHFSPRDWQRLINNNWGFRPKRLNFKLFNIQVKEVTQNEGTKTIANNLTSTVQVFTDSKYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLSRTQTNGTNATQTLLFAQAGPQNMSAQAKNWLPGPCYRQQRVSTTVSQNNNSNFTWTGATKYHLNGRDSLVNPGVAMATHKDDEERFFPSSGVLMFGKQGAGKDNVEYTNVMLTSEEEIKTTNPVATEQYGVVADNLQQTNSAPIVGAVNSQGALPGMVWQNRDVYLQGPIWAKIPHTDGNFHPSPLMGGFGLKHPPPQILIKNTPVPADPPVNFTDAKLASFITQYSTGQVSVEIEWELQKENSKRWNPEIQYTSNYYKSANVDFAVNADGVYSEPRPIGTRYLTRNL

SEQ ID NO: 14 - protein, 738 aa, adeno-associated virus
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPIGEPPAAPSSVGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLSFKLFNIQVKEVTQNEGTKTIANSLTSTIQVFTDSEYQLPYVLGSAHQGCLPPFPADVFMIPQYGYLTPNNGSQAVGRSSFYCLEYFPSQMLRTGNNFSFSYTFEDVPFHSSYAHSQSLDRLMNPLIDQYLYYLSRTQSTGGTAGTQQLLSSQAGPSNMSAQARNWLPGPCYRQQRVSTTLSQNNNSNFAWTGATKYHLNGRDSLVNPGVAMATNKDDEDRFFPSSGILMFGKQGAGKDNVDYSNVMLTSEEEIKTTNPVATEQYGVVADNLQQQNTAPIVGAVNSQGALPGMVWQNRDVYLQGPIWAKIPHTDGNFHPSPLMGGFGLKHPPPQILIKNTPVPADPPTAFNQAKLNSFITQYSTGQVSVEIEWELQKENSKRWNPEIQYTSNYYKSTNVDFAVNTEGVYSEPRPIGTRYLTRNL

SEQ ID NO: 15 - protein, 738 aa, adeno-associated virus
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPIGEPPAAPSSVGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFSFSYTFEDVPFHSSYVHSQSLDRLMNPLIDQYLYYLSRTQSTGGTAGTQQLLFSQAGPSNMSAQARNWLPGPCYRQQRVSTTLSQNNNSNFAWTGATKYHLNGRDSLVNSGVAMATNKDDEDRFFPSSGILMFGKQGAGKDNVDYSNVMLTSEEEIKTTNPVATEQYGVVADNLQQQNTAPIVGAVNSQGALPGMVWQNRDVYLQGPIWAKIPHTDGNFHPSPLMGGFGLKHPPPQILIKNTPVPADPPTAFNQAKLNSFITQYSTGQVSVEIEWELQKENSKRWNPEIQYTSNYYKSTNVDFAVNTEGVYSEPRPIGTRYPTRNL

SEQ ID NO: 16 - protein, 738 aa, adeno-associated virus
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEPSPQRSPDSSTGIGKKGQQPARKRLNFGRTGDSESVPDPQPIGEPPAAPSSVGSGTMAAGGGAPMADNNEGADGVGSSSGNWHCDSTWLGDRVITTSTRTWALPTYNNHLYKQISNGTSGGSTNDNTYFGYSTPWGYLDFNRFHCHFSPRDWQRLINNNWGFRPKRLSFKLFNIQVKEVTQNEGTKTIANNLASTIQVFTDSEYQPPYVLGSAHQGCLPPFPADVFMIPQYGYLTLNNGSQAVGRSSFYCLEYFPSQMLRTGNNFSFSYTFEDVPFHSSYAHSQSLDRLMNPLIDQYLYYLSRTQSTGGTAGTQQLLFSRAGPSNMSAQARNWLPGPCYRQQRVSTTLSQNNNSNFAWTGATKYHLNGRDSLVNPGVAMATNKDDEDRFFPSSGILMFGKQGAGKDNVDYSNVMLTSEEEIKTTNPVATEQYGVVADNLQQQNTAPTVGAVNSQGALPGMVWQNRDVYLQGPIWAKIPHTDGNFHPSPLMGGFGLKHPPPQILIKNTPVPADPPTAFNQAKLNSFIAQYSTGQVSVEIEWELQKENSKRWNPEIQYTSNYYKSTNADFAVNTEGVYSEPRPIGTRYLTRNL

SEQ ID NO: 17 - protein, 738 aa, adeno-associated virus
MAADGYLPDWLEDNLSEGIREWWDLKPGAPKPKANQQKQDDGRGLVLPGYKYLGPFNGLDKGEPVNAADAAALEHDKAYDQQLKAGDNPHLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQLIGEPPAAPSSVGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGNLTLNNGSQAVGRSSFYCLEYFPSQMLRTGNNFSFSYTFEDVPFHSSYAHSQSLDRLMNPLIDQYLYYLSRTQSTGGTAGTQQLLFSQAGPSNMSAQARNWLPGPCYRQQRVSTTLSQNNNSNFAWTGATKYHLNGRDSLVNPGVAMATNKDDEDRFFPSSGILMFGKQGAGKDNMGYSNVMLTSEEEIKTTNPVATEQYGVVADNLQQQNTAPIVGAVNSQGALPGMVWQNRDVYLQGPIWAKIPHTDGNFHPSPLMGGFGLKHPPPQILIKNTPVPADPPTAFNQAKLNSFITQYGTGQVSVEIEWELQKENSKRWNPEIQYTSNYYKSTNVDFAVNTEGVYSEPRPIGTRYLTRNL

SEQ ID NO: 18 - protein, 738 aa, adeno-associated virus
MVADGYLPDWLEDNLSEGIREWWDLKPGAPKPKANQQKQGDGRGLVLPGYKYLGPFNGLDKGEPVNAADAAALEHDKAYDQQLKAGDNPYLRYNHADAELQERLQEDTSFGGNLGRAVFQAKKRVLEPLGLVEEGAKTAPGKKRPVEPSPQRSPDSSTGIGKKGQQPARKRLNFGQTGDSESVPDPQPIGEPPAAPSSVGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCQPPFPADVFMIPQYGYLTLNNGSQAVGRSSFYCLEYFPSQMLRTGNNFSFSYTFEDVPFHSSYAHSQSLDRLMNPLIDQYLYYLSRTQSTGGTAGTQQLLFSQAGPSNMSAQARNWLPGPCYRQQRVSTTLSQNNNSNFAWTGATKYHLNGRDSLVNPGVAMATNKDDEDRFFPSSGILMFGKQGAGKDNVDYSNVMLTSEEEIKTTNPVATEQYGVVADNLQQQNTAPIVGAVNSQGALPGMVWQNRDVYLQGPIWAKIPHTDGNFHPSPLMGGFGLKHPPPQILIKNTPVPADPPTAFNQAKLNSFITQYSTGQVSVEIEWEPQKENSKRWNPEIQYTSNYYKSTNVDFAVNTEGVYSEPRPIGTRYLTRNL

SEQ ID NO: 19 - protein, 738 aa, adeno-associated virus
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPIGEPPAAPSSVGSGTMAAGGGAPMADNNEGADGVGSSSGNWHCDSTWLGDRVITTSTRTWALPTYNNHLYKQTSNGTSGGSTNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFSFSYTFEDVPFHSSYAHSQSLDRLMNPLIDQYLYYLSRTQSTGGTAGTQQLLFSQAGPSNMSAQARNWLPGPCYRQQRVSTTLSQNNNSNFAWTGATKYHLNGRDSLVNPGVAMATNKDDEDRFFPSSGILMFGKQGAGKDNVDYSNVMLTSEEEIKTTNPVATEQYGVVADNLQQQNTAPIVGAVNSQGALPGMVWQNRDVYLQGPIWAKIPHTDGNFHPSPLMGGFGLKHPPPQILIKNTPVPADPPTAFNQAKLNSFITQYSTGQVSAEIEWELQKENSKRWNPEIQYTSNYYKSTNVDFAVNTEGVYSEPRPIGTRYLTRNL

SEQ ID NO: 20 - protein, 738 aa, adeno-associated virus
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEAAKTAPGKKRPVEPSPQRSPDSSTGIGKKGQQPARKRLNFGQTGDSESVPDPQPIGEPPAAPSSVGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFSFSYTFEDVPFHSSYAHSQSLDRLMNPLIDQYLYYLSRTQSTGGTAGTQQLLFSQAGPSNMSAQARNWLPGPCYRQQRVSTTLSQNNNSNFAWTGATKYHLNGRDSLVNPGVAMATNKDDEDRFFPSSGILMFGKQGAGKDNVDYSNVMLTSEEEIKTTNPVATEQYGVVADNLQQQNTAPIVGAVNSQGALPGMVWQNRDVYLQGPIWAKIPHTDGNFHPSPLMGGFGLKHPPPQILIKSTPVPADPPTAFNQAKLNSFITQYSTGQVSVEIEWELQKENSKCWNPEIQYTSNYYKSTNVDFAVNTEGVYSEPRPIGTRYLTRNL

SEQ ID NO: 21 - protein, 738 aa, adeno-associated virus
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPIGEPPAAPSSVGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRPINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFSFSYPFEDVPFHSSYAHSQSLDRLMNPLIDQYLYYLSRTQSTGGTAGTQQLLFSQAGPSNMSAQARNWLPGPCYRQQRVSTTLSQNNNSNFAWTGATKYHLNGRDSLVNPGVAMATNKDDEDRFFPSSGILMFGKQGAGKDNVDYSNVMLTSEEEIKTTNPVATEQYGVVADNLQQQDTAPIVGAVNSQGALPGMVWQNRDVYLQGPIWAKIPHTDGNFHPSPLMGGFGLKHPPPQVLIKNTPVPADPPTAFNQAKLNSFITQYSTGQVSVEIEWELQKENSKRWNPEIQYTSNYYKSTNVDFAVNTEGVYSEPRPIGTRYLTRNL

SEQ ID NO: 22 - protein, 738 aa, adeno-associated virus
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEPSPQRSPDSSTGIGKKGQQPAGKRLNFGQTGDSESVPDPQPIGEPPAAPSSVGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFSFSYTFEDVPFHSSYAHSQSLDRLMNPLVDQYLYYLSRTQSTGGTAGTQQLLFSQAGPSNMSAQARNWLPGPCYRQQRVSTTLSQNNNSNFAWTGATKYHLNGRDSLVNPGVAMATNKDDEDRFFPSSGILMFGKQGAGKDNVDYSNVMLTSEEEIKTTNPVATEQYGVVADNLQQQNTAPIVGAVNSQGALPGMVWQNRDVYLQGPIWAKIPHTDGNFHPSPLMGGFGLKHPPPQILIKNTPVPADPPTAFNQAKLNSFITQYSTGQVSVEIEWELQKENSKRWSPEIQYTSNYYKSTNVDFAVNTEGVYSEPRPIGTRYLTRNL

SEQ ID NO: 23 - protein, 736 aa, adeno-associated virus
MAADGYLPDWLEDNLSEGIREWWDLKPGAPKPKANQQKQDDGRGLVLPGYKYLGPFNGLDKGEPVNAADAAALEHDKAYDQQLEAGDNPYLRYNHADAEFQERLQEDTSFGGNLGRAVFQAKKRVLEPLGLVEEGAKTAPGKKRPVEQSPQEPDSSSGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPVTGSCFWQQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 24 - protein, 738 aa, adeno-associated virus
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEAAKTAPGKKRPVEPSPQRSPDSSAGIGKKGQQPAKKRLNFGQTGDSESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLSFKLFNIQVKEVTQNEGTETIANNLTSTIQVFTDSEYQLPYVLGSAHQGCLPPFPADVFMIPQYGYLTLNNGSQAVGRSSFYCLEYFPSQMLRTGNNFEFSYTFEDVPFHSSCAHSQSSDRLMNPLIDQYLYYLSRTRSTGGTQGTQQLLFSQAGPANMSAQAKNWLPGPCYRQQRVSTTLSQNNNSNFAWTGATKYHLNGRDSLVNPGVAMATHKDDEERFFPSSGVLMFGKQGAGRDNVDYSSVMLTSEEEIKTTNPVATEQYGVVADNLQQTNTGPIVGNVNSQGALPGMVWQNRDVYLQGPIWAKIPHTDGNFHPSPLMGGFGLKHPPPQILIKNTPVPADPPTTFSQAKLASFITQYSTGQVSVEIEWELQKENSKRWNPEIQYTSNYYKSTNVDFAVNTEGTYSEPRPIGTRYLTRNL

SEQ ID NO: 25 - protein, 738 aa, adeno-associated virus
MAADGYLPDWLEDNLSEGIREWWDLKPGAPKPKANQQKQDDGRGLVLLGYKYLGPFNGLDKGEPVNAADAAALEHDKAYDQQLKAGDNPYLRYNHADAEFQERLQEDTSFGGNLGRAVFQAKKRVLEPLGLVEEAAKTAPGKKRPVEPSPQRSPDSSTGIGKKGQQPAKKRLNFGQTGDSESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYTFEDVPFHSGYAHSQSLDRLMNPLIDQYLYYLSRTQSTGGTQGTQQLLFSQAGPANMSAQAKNWLPGPCYRQQRVSTTLSQNNNSNFAWTGATKYHLNGRDSLVNPGVAMATHKDDEERFFPSSGVLMFGKQGAGRDNVDYSSVMLTSEEEIKTTNPVATEQYGVVADNLQQTNTGPIVGNVNSQGALPGMVWQNRDVYLQGPIWAKIPHTDGNFHPSPLMGGFGLKHPPPQILIKNTPVPADPPTTFSQAKLASFITQYSTGQVSVEIEWELQKENSKRWNPEIQYTSNYYKSTNVDFAVNTEGTYSEPRPIGTRYLTRNL

SEQ ID NO: 26 - protein, 738 aa, adeno-associated virus
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPIGEPPAAPSSVGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFSFSYTFEDVPFHSSYAHSQSLDRLMNPLIDQYLYYLSRTQSTGGTAGTQQLLFSQAGPSNMSAQARNWLPGPCYRQQRVSTTLSQNNNSNFAWTGATKYHLNGRDSLVNPGVAMATNKDDEDRFFPSSGILMFGKQGAGKDNVDYSNVMLTSEEEIKTTNPVATEQYGVVADNLQQQNTAPIVGAVNSQGALPGMVWQNRDVYLQGPIWAKIPHTDGNFHPSPLMGGFGLKHPPPQILIKNTPVPADPPTAFNQAKLNSFITQYSTGQVSVEIVWELQKENSKRRNPEIQYTSNYYKSTNVDFAVNTEGVYSEPRPIGTRYLTRNL

SEQ ID NO: 27 - protein, 738 aa, adeno-associated virus
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEPSPQRSPDSSTGIGKKGQQPAKKRLNFGQTGDSESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYQFEDVPFHSSYAHSQSLDRLMNPLIDQYLYYLSRTQSTGGTAGTQQLLFSQAGPNNMSAQAKNWLPGPCYRQQRVSTTLSQNNNSNFAWTGATKYHLNGRDSLVNPGVAMATHKDDEERFFPSSGVLMFGKQGAGKDNVDYSSVMLTSEEEIKTTNPVATEQYGVVADNLQQQNAAPIVGAVNSQGALPGMVWQNRDVYLQGPIWAKIPHTDGNFHPSPLMGGFGLKHPPPQILIKNTPVPADPPTTFSQAKLASFITQYSTGQVSVEIEWELQKENSKRWNPEIQYTSNYYKSTNVDFAVNTDGTYSEPRPIGTRYLTRNL

SEQ ID NO: 28 - protein, 4 aa, synthetic construct
QQQN

SEQ ID NO: 29 - protein, 4 aa, synthetic construct
QQTN

SEQ ID NO: 30 - protein, 4 aa, synthetic construct
QQQD

SEQ ID NO: 31 - protein, 4 aa, synthetic construct
QQAN

SEQ ID NO: 32 - protein, 748 aa, synthetic construct
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNLADQDYTKTATAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 33 - protein, 748 aa, synthetic construct
MAADGYLPDWLEDNLSEGIREWWDLKPGAPKPKANQQKQDDGRGLVLPGCKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEPSPQRSPDSSTGIGKTGQQPAKKRLNFGQTGDSESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTQNEGTKTIANNLTSTIQVFTDSEYQLPYVLGSAHQGCPPPFPADVFMIPQYGYLTLNNGSQAVGRSSFYCLEYFPSQMRRTGNNFEFSYQFEDVPFHSSYAHSQSLDRLMNPLIDQYLYYLSRTQSTGGTAGTQQLLFSQAGPNNMSAQAKNWLPGPCYRQQRVSTTLSQNNNSNFAWTGATKYHLNGRDSLVNPGVAMATHKDDEERFFPSSGVLMFGKQGAGKDNVDYSSVMLTSEEEIKTTNPVATEQYGVVADNLQQQNLADQDYTKTAAAPIVGAVNSQGALPGMVWQNRDVYLQGPIWAKIPHTDGNFHPSPLMGGFGLKHPPPQILIKNTPVPADPPTTFSQAKLASFITQYSTGQVSVEIEWELQKENSKRWNPEIQYTSNYNKSVNVDFTVDTNGVYSEPRPIGTRYLTRNL

SEQ ID NO: 34 - protein, 748 aa, synthetic construct
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEPSPQRSPDSSTGIGKTGQQPAKKRLNFGQTGDSESVPDPQPIGEPPAGPSGLGSGTMAAGGGAPMADNNEGADGVGSSSGNWHCDSAWLGDRVITTSTRPWALPTYNNHLYKQISNGTSGGSTNDNTYFGYSTPWGYFDFNRFHCHFSPRDWQRLINNNWGFRPKRLNFKLFNIQVKEVTQNEGTKTIANNLTSTIQVFTDSEYQLPYVLGSAHQGCPPPFPADVFMIPQYGYLTLNNGSQAVGRSSFYCLEYFPSQMRRTGNNFEFSYQFEDVPFHSSYAHSQSLDRLMNPLIDQYLYYLSRTQSTGGTAGTQQLLFSQAGPNNMSAQAKNWLPGPCYRQQRVSTTLSQNNNSNFAWTGATKYHLNGRDSLVNPGVAMATHKDDEERFFPSSGVLMFGKQGAGKDNVDYSSVMLTSEEEIKTTNPVATEQYGVVADNLQQQNLADQDYTKTAAAPIVGAVNSQGALPGMVWQNRDVYLQGPIWAKIPHTDGNFHPSPLMGGFGLKHPPPQILIKNTPVPADPPTTFSQAKLASFITQYSTGQVSVEIEWELQKENSKRWNPEIQYTSNYYKSTNVDFAVNTEGTYSEPRPIGTRYLTRNL

SEQ ID NO: 35 - protein, 748 aa, synthetic construct
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEAAKTAPGKKRPVEPSPQRSPDSSTGIGKKGQQPAKKRLNFGQTGDSESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYTFEDVPFHSSYAHSQSLDRLMNPLIDQYLYYLSRTQSTGGTQGTQQLLFSQAGPANMSAQAKNWLPGPCYRQQRVSTTLSQSNNSNFAWTGATKYHLNGRDSLVNPGVAMATHKDDEERFFPSSGVLMFGKQGAGRDNVDYSSVMLTSEEEIKTTNPVATEQYGVVADNLQQTNLADQDYTKTATGPIVGNVNSQGALPGMVWQNRDVYLQGPIWAKIPHTDGNFHPSPLMGGLGLKHPPPQILIKNTPVPADPPTTFSQAKLASFITQYSTGQVSVEIEWELQKENSKRWNPEIQYTSNYYKSTNVDFAVNTEGTYSEPRPIGTRYLTRNL

SEQ ID NO: 36 - protein, 748 aa, synthetic construct
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEAAKTAPGKKRPVEPSPQRSPDSSTGIGKKGQRPAKKRLNFGQTGDSESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLSFKPFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYTFEDVPFHSSYAHSQSLDRLMNPLIDQYLHYLSRTQSTGGTQGTQQLLFSQAGPANMSAQAKNWLPGPCYRQQRVSTTLSQNNNSNFAWTGATKYHLNGRDSLVNPGVAMATHKDDEERFFPSSGVLMFGKQGAGRDNVDYSSVMLTSEEEIKTTNPVATEQYGVVADNLQQTNLADQDYTKTATGPIVGNVNSQGALPGMVWQNRDVYLQGPIWAKIPHTDGNFHPSPLMGGFGLKHPPPQILIKNTPVPADPPTTFSQAKLASFITQYSTGQVSVEIEWELQKENSKRWNPEIQYTSNYYKSTNVDFAVNTEGTYSEPRPIGTRYLTRNL

SEQ ID NO: 37 - protein, 748 aa, synthetic construct
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEAAKTAPGKKRPVEPSPQRSPDSSTGIGKKGQQPAKKRLSFGQTGDSESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNSEFSYTFEDVPFHSSYAHSQSLDRLMNPLIDQYLYYLSRTQSTGGTQGTQQLLFSQAGPANMSAQAKNWLPGPCYRQQRVSTTLSQNNNSNFAWTGATKYHLNGRDSLVNPGVAMATHKDDEERFFPSSGVLMFGKQGAGRDNVDYSSVMLTSEEEIKTTNPVATEQYGVVADNLQQTNLADQDYTKTATGPIVGNVNSQGALPGMVWQNRDVYLQGPIWAKIPHTDGNFHPSPLMGGFGLKHPPPQILIKNTPVPADPPTTFSQAKLASFITQYSTGQVSVEIEWELQKENSKRWNPEIQYTSNYYKSTNVDFAVNTEGTYSEPRPIGTRYLTRNL

SEQ ID NO: 38 - protein, 748 aa, synthetic construct
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEAAKTAPGKKRPVEPSPQRSPDSSTGIGKKGQQPAKKRLNFGQTGDSESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYTFEDVPFHSSYAHSQSLDRLMNPLIDQYLYYLSRTQSTGGTQGTQQLLFSQAGPANMSAQAKNWLPGPCYRQQRVSTTLSQNNNSNFAWTGATKYHLNGRDSLVNPGVAMATHKDDEERFFPSSGVLMFGKQGAGRDNVDYSSVMLTSEEEIKTTNPVATEQYGVVADNLQQTNLADQDYTKTATGPIVGNVNSQGALPGMVWQNRDVYLQGPIWAKIPHTDGNFHPSPLMGGFGLKHPPPQILIKNTPVPADPPTTFSQAKLASFITQYSTGQVSVEIEWELQKENSKRWNPEIQYTSNYYKSTNVDFAVNTEGTYSEPRPIGTRYLTRNL

SEQ ID NO: 39 - protein, 748 aa, synthetic construct
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGPVEEAAKTAPGKKRPVEPPPQRSPDSSTGIGKKGQQPAKKRLNFGQTGDSESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLSFKLFNIQVKEVTQNEGTKTVANNLTSTIQVFTDSEYQLPYVLGSAHQGCLPPFPADVFMIPQYGYLTLNNGSQAVGRSSFYCLEYFPSQMLRTGNNFEFSYTFEDVPFHSSYAHSQSLDRLMNPLIDQYLYYLSRTQSTGGTQGTQQLLFSQAGPANMSAQAKNWLPGPCYRQQRVSTTLSQNNNSNFAWTGATKYHLNGRDSLVNPGVAMATHKDDEERFFPSSGVLMFGKQGAGRDNVDYSSVMLTSEEEIKTTNPVATEQYGVVADNLQQTNLADQDYTKTATGPIVGNVNSQGALPGMVWQNRDVYLQGPIWAKIPHTDGNFHPSPLMGGFGLKHPPPQILIKNTPVPADPPTTFSQAKLASFITQYSTGQVSVEIEWELQKENSKRWNPEIQYTSNYYKSTNVDFAVNTEGTYSEPRPIGTRYLTRNL

SEQ ID NO: 40 - protein, 748 aa, synthetic construct
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEAAKTAPGKKRPVEPSPQRSPDSSTGIGKKGQQPAKKRLNFGQTGDSESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLSFKLFNIQVKEVTQDEGTKTIANNLTSTIQVFTDSEYQLPYVLGSAHQGCLPPFPADVFMIPQYGYLTLNNGSQAVGRSSFYCLEYFPSQMLRTGNNFEFSYTFEDVPFHSSYAHSQSLDRLMNPLIDQYLYYLSRTQSTGGTQGTQQLLFSQAGPANMSARAKNWLPGPCYRQQRVSTTLSQNNNSNFAWTGATKYHLNGRDSLVNPGVAMATHKDDEERFFPSSGVLMFGKQGAGRDNVDYSSVMLTSEEEIKTTNPVATEQYGVVADNLQQANLADQDYTKTATGPIVGNVNSQGALPGMVWQNRDVYLQGPIWAKIPHTDGNFHPSPLMGGFGLKHPPPQILIKNTPVPADPPTTFSQAKLASFITQYSTGQVSVEIEWELQKENSKRWNPEIQYTSNYYKSTNVDFAVNSEGTYSEPRPIGTRYLTRNL

SEQ ID NO: 41 - protein, 741 aa, synthetic construct
MAADGYLPDWLEDNLSEGIREWWALKPGAPQPKANQQKQDDGRGLVLPGYKYLGPFNGLDKGEPVNEADAAALEHDKAYDQQLKAGDNPYLRYNHADAEFQERLQEDTSFGGNLGRAVFQAKKRVLEPLGLVEEGAKTAPGKKRPVEPDSSSGIGKSGQQPAKKRLNFGQTGDSESVPDPQPLSEPPAGPSGLGSGTMAAGGGAPMADNNEGADGVGNVSGNWHCDSTWLGDRVITTSTRTWALPTYNNHLYKQISNGTSGGSSNDNTYFGYSTPWGYFDFNRFHCHFSPRDWQRLINNNWGFRPKKLNFKLFNIQVKEVTQNEGTKTIANNLTSTVQVFTDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLSRTQTNGTNATQTLLFAQAGPQNMSAQAKNWLPGPCYRQQRVSTTVSQNNNSNFTWTGATKYHLNGRDSLVSPGVAMATHKDDEERFFPSSGVLMFGKQGAGKDNVEYTNVMLTSEEEIKTTNPVATEQYGVVADNLQQTNLADQDYTKTASAPIVGAVNSQGALPGMVWQNRDVYLQGPIWAKIPHTDGNFHPSPLMGGFGLKHPPPQILIKNTPVPADPPVNFTDAKLASFITQYSTGQVSVEIEWELQKENSKRWNPEIQYTSNYYKSANVDFAVNADGVYSEPRPIGTRYLTRNL

SEQ ID NO: 42 - protein, 741 aa, synthetic construct
MAADGYLPDWLEDNLSEGIREWWALKPGAPQPKANQQKQDDGRGLVLPGYKYLGPFNGLDKGEPVNEADAAALEHDKAYDQQLKAGDNPYLRYNHADAEFQERLQEDTSFGGNLGRAVFQAKKRVLEPLGLVEEGAKTAPGKKRPVEPDSSSGIGKSGQQPAKKRLNFGPTGDSESVPDPQPLSEPPAGPSGLGSGTMAAGGGAPMADNNEGADGVGNVSGNWHCDSTWLGDRVITTSTRTWALPTYNNHLYKQISNGTSGGSSNDNTYFGYSTPWGYFDFNRFHCHFSPRDWQRLINNNWGFRPKKLNFKLFNIQVKEVTQNEGTKTTANNLTSTVQVFTDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLSRTQTNGTNATQTLLFAQAGPQNMSAQAKNWLPGPCYRQQRVSTAVSQNNNSNFTWTGATKYHLNGRDSLVNPGVAMATHKDDEERFFPSSGVLMFGKQGAGKDNVEYTNVMLTSEEEIKTTNPVATEQYGVVADNLQQTNLADQDYTKTASAPIVGAVNSQGALPGMVWQNRDVYLQGPIWAKIPHTDGNFHPSPLMGGFGLKHPPPQILIKNTPVPADPPVNFTDAKLASFITQYSTGQVSVEIEWELQKENSKRWNPEIQYTSNYYKSANVDFAVNADGVYSEPRPIGTRYLTRNL

SEQ ID NO: 43 - protein, 741 aa, synthetic construct
MAADGYLPDWLEDNLSEGIREWWALKPGAPQPKANQQKQDDGRGLVLPGYKYLGPFNGLDKGEPVNEADAAALEHDKAYDQQLKAGDNPYLRYNHADAEFQERLQEDTSFGGNLGRAVFQAKKRVLEPLGLVEEGAKTAPGKKRPVEPDSSSGIGKSGRQPAKKRLNFGQTGDSESVPDPQPLSEPPAGPSGLGSGTMAAGGGAPMADNNEGADGVGNASGNWHCDSTWLGDRVITTSTRTWALPTYNNHLYKQISNGTSGGSSNDNTYFGYSTPWGYFDFNRFHCHFSPRDWQRLINNNWGFRPKRLNFKLFNIQVKEVTQNEGTKTIANNLTSTVQVFTDSKYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLSRTQTNGTNATQTLLFAQAGPQNMSAQAKNWLPGPCYRQQRVSTTVSQNNNSNFTWTGATKYHLNGRDSLVNPGVAMATHKDDEERFFPSSGVLMFGKQGAGKDNVEYTNVMLTSEEEIKTTNPVATEQYGVVADNLQQTNLADQDYTKTASAPIVGAVNSQGALPGMVWQNRDVYLQGPIWAKIPHTDGNFHPSPLMGGFGLKHPPPQILIKNTPVPADPPVNFTDAKLASFITQYSTGQVSVEIEWELQKENSKRWNPEIQYTSNYYKSANVDFAVNADGVYSEPRPIGTRYLTRNL

SEQ ID NO: 44 - protein, 748 aa, synthetic construct
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPIGEPPAAPSSVGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLSFKLFNIQVKEVTQNEGTKTIANSLTSTIQVFTDSEYQLPYVLGSAHQGCLPPFPADVFMIPQYGYLTPNNGSQAVGRSSFYCLEYFPSQMLRTGNNFSFSYTFEDVPFHSSYAHSQSLDRLMNPLIDQYLYYLSRTQSTGGTAGTQQLLSSQAGPSNMSAQARNWLPGPCYRQQRVSTTLSQNNNSNFAWTGATKYHLNGRDSLVNPGVAMATNKDDEDRFFPSSGILMFGKQGAGKDNVDYSNVMLTSEEEIKTTNPVATEQYGVVADNLQQQNLADQDYTKTATAPIVGAVNSQGALPGMVWQNRDVYLQGPIWAKIPHTDGNFHPSPLMGGFGLKHPPPQILIKNTPVPADPPTAFNQAKLNSFITQYSTGQVSVEIEWELQKENSKRWNPEIQYTSNYYKSTNVDFAVNTEGVYSEPRPIGTRYLTRNL

SEQ ID NO: 45 - protein, 748 aa, synthetic construct
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPIGEPPAAPSSVGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFSFSYTFEDVPFHSSYVHSQSLDRLMNPLIDQYLYYLSRTQSTGGTAGTQQLLFSQAGPSNMSAQARNWLPGPCYRQQRVSTTLSQNNNSNFAWTGATKYHLNGRDSLVNSGVAMATNKDDEDRFFPSSGILMFGKQGAGKDNVDYSNVMLTSEEEIKTTNPVATEQYGVVADNLQQQNLADQDYTKTATAPIVGAVNSQGALPGMVWQNRDVYLQGPIWAKIPHTDGNFHPSPLMGGFGLKHPPPQILIKNTPVPADPPTAFNQAKLNSFITQYSTGQVSVEIEWELQKENSKRWNPEIQYTSNYYKSTNVDFAVNTEGVYSEPRPIGTRYPTRNL

SEQ ID NO: 46 - protein, 748 aa, synthetic construct
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEPSPQRSPDSSTGIGKKGQQPARKRLNFGRTGDSESVPDPQPIGEPPAAPSSVGSGTMAAGGGAPMADNNEGADGVGSSSGNWHCDSTWLGDRVITTSTRTWALPTYNNHLYKQISNGTSGGSTNDNTYFGYSTPWGYLDFNRFHCHFSPRDWQRLINNNWGFRPKRLSFKLFNIQVKEVTQNEGTKTIANNLASTIQVFTDSEYQPPYVLGSAHQGCLPPFPADVFMIPQYGYLTLNNGSQAVGRSSFYCLEYFPSQMLRTGNNFSFSYTFEDVPFHSSYAHSQSLDRLMNPLIDQYLYYLSRTQSTGGTAGTQQLLFSRAGPSNMSAQARNWLPGPCYRQQRVSTTLSQNNNSNFAWTGATKYHLNGRDSLVNPGVAMATNKDDEDRFFPSSGILMFGKQGAGKDNVDYSNVMLTSEEEIKTTNPVATEQYGVVADNLQQQNLADQDYTKTATAPTVGAVNSQGALPGMVWQNRDVYLQGPIWAKIPHTDGNFHPSPLMGGFGLKHPPPQILIKNTPVPADPPTAFNQAKLNSFIAQYSTGQVSVEIEWELQKENSKRWNPEIQYTSNYYKSTNADFAVNTEGVYSEPRPIGTRYLTRNL

SEQ ID NO: 47 - protein, 748 aa, synthetic construct
MAADGYLPDWLEDNLSEGIREWWDLKPGAPKPKANQQKQDDGRGLVLPGYKYLGPFNGLDKGEPVNAADAAALEHDKAYDQQLKAGDNPHLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQLIGEPPAAPSSVGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGNLTLNNGSQAVGRSSFYCLEYFPSQMLRTGNNFSFSYTFEDVPFHSSYAHSQSLDRLMNPLIDQYLYYLSRTQSTGGTAGTQQLLFSQAGPSNMSAQARNWLPGPCYRQQRVSTTLSQNNNSNFAWTGATKYHLNGRDSLVNPGVAMATNKDDEDRFFPSSGILMFGKQGAGKDNMGYSNVMLTSEEEIKTTNPVATEQYGVVADNLQQQNLADQDYTKTATAPIVGAVNSQGALPGMVWQNRDVYLQGPIWAKIPHTDGNFHPSPLMGGFGLKHPPPQILIKNTPVPADPPTAFNQAKLNSFITQYGTGQVSVEIEWELQKENSKRWNPEIQYTSNYYKSTNVDFAVNTEGVYSEPRPIGTRYLTRNL

SEQ ID NO: 48 - protein, 748 aa, synthetic construct
MVADGYLPDWLEDNLSEGIREWWDLKPGAPKPKANQQKQGDGRGLVLPGYKYLGPFNGLDKGEPVNAADAAALEHDKAYDQQLKAGDNPYLRYNHADAELQERLQEDTSFGGNLGRAVFQAKKRVLEPLGLVEEGAKTAPGKKRPVEPSPQRSPDSSTGIGKKGQQPARKRLNFGQTGDSESVPDPQPIGEPPAAPSSVGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCQPPFPADVFMIPQYGYLTLNNGSQAVGRSSFYCLEYFPSQMLRTGNNFSFSYTFEDVPFHSSYAHSQSLDRLMNPLIDQYLYYLSRTQSTGGTAGTQQLLFSQAGPSNMSAQARNWLPGPCYRQQRVSTTLSQNNNSNFAWTGATKYHLNGRDSLVNPGVAMATNKDDEDRFFPSSGILMFGKQGAGKDNVDYSNVMLTSEEEIKTTNPVATEQYGVVADNLQQQNLADQDYTKTATAPIVGAVNSQGALPGMVWQNRDVYLQGPIWAKIPHTDGNFHPSPLMGGFGLKHPPPQILIKNTPVPADPPTAFNQAKLNSFITQYSTGQVSVEIEWEPQKENSKRWNPEIQYTSNYYKSTNVDFAVNTEGVYSEPRPIGTRYLTRNL

SEQ ID NO: 49 - protein, 748 aa, synthetic construct
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPIGEPPAAPSSVGSGTMAAGGGAPMADNNEGADGVGSSSGNWHCDSTWLGDRVITTSTRTWALPTYNNHLYKQTSNGTSGGSTNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFSFSYTFEDVPFHSSYAHSQSLDRLMNPLIDQYLYYLSRTQSTGGTAGTQQLLFSQAGPSNMSAQARNWLPGPCYRQQRVSTTLSQNNNSNFAWTGATKYHLNGRDSLVNPGVAMATNKDDEDRFFPSSGILMFGKQGAGKDNVDYSNVMLTSEEEIKTTNPVATEQYGVVADNLQQQNLADQDYTKTATAPIVGAVNSQGALPGMVWQNRDVYLQGPIWAKIPHTDGNFHPSPLMGGFGLKHPPPQILIKNTPVPADPPTAFNQAKLNSFITQYSTGQVSAEIEWELQKENSKRWNPEIQYTSNYYKSTNVDFAVNTEGVYSEPRPIGTRYLTRNL

SEQ ID NO: 50 - protein, 748 aa, synthetic construct
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEAAKTAPGKKRPVEPSPQRSPDSSTGIGKKGQQPARKRLNFGQTGDSESVPDPQPIGEPPAAPSSVGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFSFSYTFEDVPFHSSYAHSQSLDRLMNPLIDQYLYYLSRTQSTGGTAGTQQLLFSQAGPSNMSAQARNWLPGPCYRQQRVSTTLSQNNNSNFAWTGATKYHLNGRDSLVNPGVAMATNKDDEDRFFPSSGILMFGKQGAGKDNVDYSNVMLTSEEEIKTTNPVATEQYGVVADNLQQQNLADQDYTKTATAPIVGAVNSQGALPGMVWQNRDVYLQGPIWAKIPHTDGNFHPSPLMGGFGLKHPPPQILIKSTPVPADPPTAFNQAKLNSFITQYSTGQVSVEIEWELQKENSKCWNPEIQYTSNYYKSTNVDFAVNTEGVYSEPRPIGTRYLTRNL

SEQ ID NO: 51 - protein, 748 aa, synthetic construct
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPIGEPPAAPSSVGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRPINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFSFSYPFEDVPFHSSYAHSQSLDRLMNPLIDQYLYYLSRTQSTGGTAGTQQLLFSQAGPSNMSAQARNWLPGPCYRQQRVSTTLSQNNNSNFAWTGATKYHLNGRDSLVNPGVAMATNKDDEDRFFPSSGILMFGKQGAGKDNVDYSNVMLTSEEEIKTTNPVATEQYGVVADNLQQQDLADQDYTKTATAPIVGAVNSQGALPGMVWQNRDVYLQGPIWAKIPHTDGNFHPSPLMGGFGLKHPPPQVLIKNTPVPADPPTAFNQAKLNSFITQYSTGQVSVEIEWELQKENSKRWNPEIQYTSNYYKSTNVDFAVNTEGVYSEPRPIGTRYLTRNL

SEQ ID NO: 52 - protein, 748 aa, synthetic construct
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEPSPQRSPDSSTGIGKKGQQPAGKRLNFGQTGDSESVPDPQPIGEPPAAPSSVGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFSFSYTFEDVPFHSSYAHSQSLDRLMNPLVDQYLYYLSRTQSTGGTAGTQQLLFSQAGPSNMSAQARNWLPGPCYRQQRVSTTLSQNNNSNFAWTGATKYHLNGRDSLVNPGVAMATNKDDEDRFFPSSGILMFGKQGAGKDNVDYSNVMLTSEEEIKTTNPVATEQYGVVADNLQQQNLADQDYTKTATAPIVGAVNSQGALPGMVWQNRDVYLQGPIWAKIPHTDGNFHPSPLMGGFGLKHPPPQILIKNTPVPADPPTAFNQAKLNSFITQYSTGQVSVEIEWELQKENSKRWSPEIQYTSNYYKSTNVDFAVNTEGVYSEPRPIGTRYLTRNL

SEQ ID NO: 53 - protein, 746 aa, synthetic construct
MAADGYLPDWLEDNLSEGIREWWDLKPGAPKPKANQQKQDDGRGLVLPGYKYLGPFNGLDKGEPVNAADAAALEHDKAYDQQLEAGDNPYLRYNHADAEFQERLQEDTSFGGNLGRAVFQAKKRVLEPLGLVEEGAKTAPGKKRPVEQSPQEPDSSSGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPVTGSCFWQQNAARDNADYSDVMLTSEEEIKTTNPVATEEYGIVADNLQQQNLADQDYTKTATAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 54 - protein, 748 aa, synthetic construct
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEAAKTAPGKKRPVEPSPQRSPDSSAGIGKKGQQPAKKRLNFGQTGDSESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLSFKLFNIQVKEVTQNEGTETIANNLTSTIQVFTDSEYQLPYVLGSAHQGCLPPFPADVFMIPQYGYLTLNNGSQAVGRSSFYCLEYFPSQMLRTGNNFEFSYTFEDVPFHSSCAHSQSSDRLMNPLIDQYLYYLSRTRSTGGTQGTQQLLFSQAGPANMSAQAKNWLPGPCYRQQRVSTTLSQNNNSNFAWTGATKYHLNGRDSLVNPGVAMATHKDDEERFFPSSGVLMFGKQGAGRDNVDYSSVMLTSEEEIKTTNPVATEQYGVVADNLQQTNLADQDYTKTATGPIVGNVNSQGALPGMVWQNRDVYLQGPIWAKIPHTDGNFHPSPLMGGFGLKHPPPQILIKNTPVPADPPTTFSQAKLASFITQYSTGQVSVEIEWELQKENSKRWNPEIQYTSNYYKSTNVDFAVNTEGTYSEPRPIGTRYLTRNL

SEQ ID NO: 55 - protein, 748 aa, synthetic construct
MAADGYLPDWLEDNLSEGIREWWDLKPGAPKPKANQQKQDDGRGLVLLGYKYLGPFNGLDKGEPVNAADAAALEHDKAYDQQLKAGDNPYLRYNHADAEFQERLQEDTSFGGNLGRAVFQAKKRVLEPLGLVEEAAKTAPGKKRPVEPSPQRSPDSSTGIGKKGQQPAKKRLNFGQTGDSESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYTFEDVPFHSGYAHSQSLDRLMNPLIDQYLYYLSRTQSTGGTQGTQQLLFSQAGPANMSAQAKNWLPGPCYRQQRVSTTLSQNNNSNFAWTGATKYHLNGRDSLVNPGVAMATHKDDEERFFPSSGVLMFGKQGAGRDNVDYSSVMLTSEEEIKTTNPVATEQYGVVADNLQQTNLADQDYTKTATGPIVGNVNSQGALPGMVWQNRDVYLQGPIWAKIPHTDGNFHPSPLMGGFGLKHPPPQILIKNTPVPADPPTTFSQAKLASFITQYSTGQVSVEIEWELQKENSKRWNPEIQYTSNYYKSTNVDFAVNTEGTYSEPRPIGTRYLTRNL

SEQ ID NO: 56 - protein, 748 aa, synthetic construct
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPIGEPPAAPSSVGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFSFSYTFEDVPFHSSYAHSQSLDRLMNPLIDQYLYYLSRTQSTGGTAGTQQLLFSQAGPSNMSAQARNWLPGPCYRQQRVSTTLSQNNNSNFAWTGATKYHLNGRDSLVNPGVAMATNKDDEDRFFPSSGILMFGKQGAGKDNVDYSNVMLTSEEEIKTTNPVATEQYGVVADNLQQQNLADQDYTKTATAPIVGAVNSQGALPGMVWQNRDVYLQGPIWAKIPHTDGNFHPSPLMGGFGLKHPPPQILIKNTPVPADPPTAFNQAKLNSFITQYSTGQVSVEIVWELQKENSKRRNPEIQYTSNYYKSTNVDFAVNTEGVYSEPRPIGTRYLTRNL

SEQ ID NO: 57 - protein, 748 aa, synthetic construct
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEPSPQRSPDSSTGIGKKGQQPAKKRLNFGQTGDSESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYQFEDVPFHSSYAHSQSLDRLMNPLIDQYLYYLSRTQSTGGTAGTQQLLFSQAGPNNMSAQAKNWLPGPCYRQQRVSTTLSQNNNSNFAWTGATKYHLNGRDSLVNPGVAMATHKDDEERFFPSSGVLMFGKQGAGKDNVDYSSVMLTSEEEIKTTNPVATEQYGVVADNLQQQNLADQDYTKTAAAPIVGAVNSQGALPGMVWQNRDVYLQGPIWAKIPHTDGNFHPSPLMGGFGLKHPPPQILIKNTPVPADPPTTFSQAKLASFITQYSTGQVSVEIEWELQKENSKRWNPEIQYTSNYYKSTNVDFAVNTDGTYSEPRPIGTRYLTRNL

SEQ ID NO: 58 - protein, 4 aa, synthetic construct
TAPQ

SEQ ID NO: 59 - protein, 4 aa, synthetic construct
SAPI

SEQ ID NO: 60 - protein, 4 aa, synthetic construct
TAPT

SEQ ID NO: 61 - protein, 4 aa, synthetic construct
TAPI

SEQ ID NO: 62 - protein, 4 aa, synthetic construct
TGPI

SEQ ID NO: 63 - protein, 4 aa, synthetic construct
AAPI

SEQ ID NO: 64 - protein, 735 aa, synthetic construct [sequence omitted]

SEQ ID NO: 65 - protein, 745 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNLADQDYTKTARQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL